(19) United States
(12) Patent Application Publication
(10) Pub. No.: US 2019/0276891 A1
(43) Pub. Date: Sep. 12, 2019

(54) GENE FOR IDENTIFYING INDIVIDUALS WITH FAMILIAL DYSAUTONOMIA

(71) Applicant: THE GENERAL HOSPITAL CORPORATION, BOSTON, MA (US)

(72) Inventors: SUSAN SLAUGENHAUPT, QUINCY, MA (US); JAMES F. GUSELLA, FRAMINGHAM, MA (US)

(21) Appl. No.: 16/169,234

(22) Filed: Oct. 24, 2018

Related U.S. Application Data
(63) Continuation of application No. 15/937,291, filed on Mar. 27, 2018, now abandoned.
Application No. 15/937,291 is a continuation of application No. 14/881,966, filed on Oct. 13, 2015, now abandoned.
Application No. 14/881,966 is a continuation of application No. 13/449,981, filed on Apr. 18, 2012, now abandoned.
Application No. 13/449,981 is a continuation of application No. 12/172,847, filed on Jul. 14, 2008, now abandoned.
Application No. 12/172,847 is a continuation of application No. 10/041,856, filed on Jan. 7, 2002, now Pat. No. 7,388,093.
(60) Provisional application No. 60/260,080, filed on Jan. 6, 2001.

(51) Int. Cl. C12Q 1/6883; C07K 14/47; C12N 15/85

(52) U.S. Cl. CPC C12Q 1/6883; C07K 14/47; C12N 15/8509; A01K 2267/0306; A01K 2267/0318; A01K 2217/075; A01K 2217/05; C12Q 2600/156; A01K 2227/105

(57) ABSTRACT

This invention relates to methods and compositions useful for detecting mutations which cause Familial Dysautonomia. Familial dysautonomia (FD; Riley-Day syndrome), an Ashkenazi Jewish disorder, is the best known and most frequent of a group of congenital sensory neuropathies and is characterized by widespread sensory and variable autonomic dysfunction. Previously, we mapped the FD gene, DYS, to a 0.5 cM region of chromosome 9q31 and showed that the ethnic bias is due to a founder effect, with >99.5% of disease alleles sharing a common ancestral haplotype. To investigate the molecular basis of FD, we sequenced the minimal candidate region and cloned and characterized its 5 genes. One of these, IKBKAP, harbors two mutations that can cause FD. The major haplotype mutation is located in the donor splice site of intron 20. This mutation can result in skipping of exon 20 in the mRNA from FD patients, although they continue to express varying levels of wild-type message in a tissue-specific manner. RNA isolated from patient lymphoblasts is primarily wild-type, whereas only the deleted message is seen in RNA isolated from brain. The mutation associated with the minor haplotype in four patients is a missense (R696P) mutation in exon 19 that is predicted to disrupt a potential phosphorylation site. Our findings indicate that almost all cases of FD are caused by an unusual splice defect that displays tissue-specific expression; and they also provide the basis for rapid carrier screening in the Ashkenazi Jewish population.

This application is a continuation under 35 U.S.C. § 120 of application Ser. No. 15/937,291, filed Mar. 27, 2018, which is a continuation of application Ser. No. 14/881,966, filed Oct. 13, 2015, which is a continuation of application Ser. No. 13/449,981, filed Apr. 18, 2012, now abandoned, which is a continuation of application Ser. No. 12/172,847, filed Jul. 14, 2008, now abandoned, which is a continuation of application Ser. No. 10/041,856, filed Jan. 7, 2002, now U.S. Pat. No. 7,388,093, which claims priority to U.S. provisional application no. 60/260,080, filed on Jan. 6, 2001, the contents of each of which are incorporated herein in their entirety.

This invention was made with government support under Grant Number NS36326 awarded by The National Institutes of Health. The U.S. government has certain rights in the invention.

### FIELD OF THE INVENTION

This invention relates generally to the gene, and mutations thereto, that are responsible for the disease familial dysautonomia (FD). More particularly, the invention relates to the identification, isolation and cloning of the DNA sequence corresponding to the normal and mutant FD genes, as well as characterization of their transcripts and gene products. This invention also relates to genetic screening methods and kits for identifying FD mutant and wild-type alleles, and further relates to FD diagnosis, prenatal screening and diagnosis, and therapies of FD, including gene therapeutics and protein/antibody based therapeutics.

### BACKGROUND OF THE INVENTION

Familial Dysautonomia (FD, Riley-Day Syndrome, Hereditary Sensory and Autonomic Neuropathy Type III) [OMIM 223900] is an autosomal recessive disorder present in 1 in 3,600 live births in the Ashkenazi Jewish population. This debilitating disorder is due to the poor development, survival, and progressive degeneration of the sensory and autonomic nervous system (Axelrod et al., 1974). FD was first described in 1949 based on five children who presented with defective lacrimation, excessive sweating, skin blotching, and hypertension (Riley et al., 1949). The following cardinal criteria have evolved for diagnosis of FD: absence of fungiform papillae on the tongue, absence of flare after injection of intradermal histamine, decreased or absent deep tendon reflexes, absence of overflow emotional tears, and Ashkenazi Jewish descent (Axelrod and Pearson, 1984, Axelrod 1984).

The loss of neuronal function in FD has many repercussions, with patients displaying gastrointestinal dysfunction, abnormal respiratory responses to hypoxic and hypercarbie states, scoliosis, gastroesophageal reflux, vomiting crises, lack of overflow tears, inappropriate sweating, and postural hypotension (Riley et al.1949; Axelrod et al.1974, Axelrod 1996). Despite recent advances in the management of FD, the disorder is inevitably fatal with only 50% of patients reaching 30 years of age. The clinical features of FD are due to a genetic defect that causes a striking, progressive depletion of unmyelinated sensory and autonomic neurons (Pearson and Pytel 1978a; Pearson and Pytel 1978b; Pearson et al. 1978; Axelrod 1995). This neuronal deficiency begins during development, as extensive pathology is evident even in the youngest subjects. Fetal development and postnatal maintenance of dorsal root ganglion (DRG) neurons is abnormal, significantly decreasing their numbers and resulting in DRG of grossly reduced size. Slow progressive degeneration is evidenced by continued neuronal depletion with increasing age. In the autonomic nervous system, superior cervical sympathetic ganglia are also reduced in size due to a severe decrease in the neuronal population.

Previously, the FD gene, DYS, was mapped to an 11-cM region of chromosome 9q31 (Blumenfeld et al. 1993) which was then narrowed by haplotype analysis to <0.5cM or 471 kb (Blumenfeld et al. 1999). There is a single major haplotype that accounts for >99.5% of all FD chromosomes in the Ashkenazi Jewish (AJ) population. The recent identification of several single nucleotide polymorphisms (SNPs) in the candidate interval has allowed for further reduction of the candidate region to 177 kb by revealing a common core haplotype shared by the major and one previously described minor haplotype (Blumenfeld et al. 1999).

### SUMMARY OF THE INVENTION

This invention relates to mutations in the IKBKAP gene which the inventors of this invention discovered and found to be associated with Familial Dysautonomia. The mutation associated with the major haplotype of FD is a base pair mutation, wherein the thymine nucleotide located at bp 6 of intron 20 in the IKBKAP gene is replaced with a cytosine nucleotide (T→C) (hereinafter “FD 1 mutation”). The mutation associated with the minor haplotype is a base pair mutation wherein the guanine nucleotide at bp 2397 (bp 73 of exon 19) is replaced with a cysteine nucleotide (G→C) (hereinafter “FD2 mutation”). This base pair mutation causes an arginine to proline missense mutation (R696P) in the amino acid sequence of the IKBKAP gene that is predicted to disrupt a potential phosphorylation site.

In accordance with one aspect of the present invention, there is provided an isolated nucleic acid comprising a nucleic acid sequence selected from the group consisting of:

nucleic acid sequences corresponding to the genomic sequence of the FD gene including introns and exons as shown in FIGS. 6-1 to 6-42;

nucleic acid sequences corresponding to the nucleic acid sequence of the FD gene as shown in FIGS. 6-1 to 6-42, wherein the thymine nucleotide at position 34,201 is replaced by a cytosine nucleotide;

nucleic acid sequences corresponding to the nucleic acid sequence of the FD gene as shown in FIGS. 6-1 to 6-42, wherein the guanine nucleotide at position 33,714 is replaced by a cytosine nucleotide;

nucleic acid sequences corresponding to the nucleic acid sequence of the FD gene as shown in FIGS. 6-1 to 6-42, wherein the thymine nucleotide at position 34,201 is replaced by a cytosine nucleotide and the guanine nucleotide at position 33,714 is replaced by a cytosine nucleotide;

nucleic acid sequences corresponding to the cDNA sequence including the coding sequence of the FD gene as shown in FIGS. 7A-7C;

nucleic acid sequences corresponding to the cDNA sequence shown in FIGS. 7A-7C, wherein the arginine at position 696 is replaced by a proline.

In accordance with another aspect of the present invention, there is provided a nucleic acid probe, comprising a nucleotide sequence corresponding to a portion of a nucleic acid as set forth in any one of the foregoing nucleic acid sequences.

In accordance with another aspect of the present invention, there is provided a cloning vector comprising a coding sequence of a nucleic acid as set forth above and a replicon operative in a host cell for the vector.

In accordance with another aspect of the present invention, there is provided an expression vector comprising a coding sequence of a nucleic acid set forth above operably linked with a promoter sequence capable of directing expression of the coding sequence in host cells for the vector.

In accordance with another aspect of the present invention, there is provided host cells transformed with a vector as set forth above.

In accordance with another aspect of the present invention, there is provided a method of producing a mutant FD polypeptide comprising: transforming host cells with a vector capable of expressing a polypeptide from a nucleic acid sequence as set forth above; culturing the cells under conditions suitable for production of the polypeptide; and recovering the polypeptide.

In accordance with another aspect of the present invention, there is provided a peptide product selected from the group consisting of: a polypeptide having an amino acid sequence corresponding to the amino acid sequence shown in FIG. 8; a polypeptide containing a mutation in the amino acid sequence shown in FIG. 8, wherein the arginine at position 696 is replaced with a proline; a peptide comprising at least 6 amino acid residues corresponding to the amino acid sequence shown in FIG. 8, and a peptide comprising at least 6 amino acid residues corresponding to a mutated form of the amino acid sequence shown in FIG. 8. In one embodiment, the peptide is labeled. In another embodiment, the peptide is a fusion protein.

In accordance with another aspect of the present invention, there is provided a use of a peptide as set forth above as an immunogen for the production of antibodies. In one embodiment, there is provided an antibody produced in such application. In one embodiment, the antibody is labeled. In another embodiment, the antibody is bound to a solid support.

In accordance with another aspect of the present invention, there is provided a method to determine the presence or absence of the familial dysautonomia (FD) gene mutation in an individual, comprising: isolating genomic DNA, cDNA, or RNA from a potential FD disease carrier or patient; and assessing the DNA for the presence or absence of an FD-associated allele, wherein said FD-associated allele is the FDI and/or FD2 mutation wherein, the absence of either FD-associated allele indicates the absence of the FD gene mutation in the genome of the individual and the presence of the allele indicates that the individual is either affected with FD or a heterozygote carrier.

In one embodiment, the assessing step is performed by a process which comprises subjecting the DNA to amplification using oligonucleotide primers flanking the FD1 mutation and the FD2 mutation. In another embodiment, the assessing step further comprises an allele-specific oligonucleotide hybridization assay.

In another embodiment, DNA is amplified using the following oligonucleotide primers: 5′-GCCAGTGTTTTTGCCTGAG-3′; 5′-CGGATTGTCACTGTTGTGC-3′; 5′-GACTGCTCTCATAGCATCGC-3′. In another embodiment, the assessing step further comprises an allele-specific oligonucleotide hybridization assay. In another embodiment, the allele-specific oligonucleotide hybridization assay is accomplished using the following oligonucleotides: 5′-AAGTAAG(T/C)GCCATTG-3′ and 5′-GGTTCAC(G/C)GATTGTC. In yet another embodiment, neuronal tissue from an individual is screened for the presence of truncated IKBKAP mRNA or peptides, wherein the presence of said truncated mRNA or peptides indicates that said individual possesses the FD1 and/or FD2 mutation in the IKBKAP gene.

In accordance with another aspect of the present invention, there is provided an animal model for familial dysautonomia (FD), comprising a mammal possessing a mutant or knock-out or knock-in FD gene. In another embodiment, there is provided a method of producing a transgenic animal expressing a mutant IKAP mRNA comprising:

(a) introducing into an embryonal cell of an animal a promoter operably linked to the nucleotide sequence containing a mutation associated with FD;

(b) transplanting the transgenic embryonal target cell formed thereby into a recipient female parent; and

(c) identifying at least one offspring containing said nucleotide sequence in said offspring's genome.

In accordance with another aspect of the present invention, there is provided a method for screening potential therapeutic agents for activity, in connection with FD, comprising: providing a screening tool selected from the group consisting of a cell line, and a mammal containing or expressing a defective FD gene or gene product; contacting the screening tool with the potential therapeutic agent; and assaying the screening tool for an activity.

In accordance with another aspect of the present invention, there is provided a method for treating familial dysautonomia (FD) by gene therapy using recombinant DNA technology to deliver the normal form of the ED gene into patient cells or vectors which will supply the patient with gene product in vivo.

In another embodiment, there is provided a method for treating familial dysautonomia (FD), comprising: providing an antibody directed against an FD protein sequence or peptide product; and delivering the antibody to affected tissues or cells in a patient having FD.

In accordance with another aspect of the present invention, there is provided kits for carrying out the methods of the invention. These kits include nucleic acids, polypeptides and antibodies of the present invention. In another embodiment the kit for detecting FD mutations will also contain genetic tests for diagnosing additional genetic diseases, such as Canavan's disease, Tay-Sachs disease, Goucher disease, Cystic Fibrosis, Fanconi anemia, and Bloom syndrome.

It will be appreciated by a skilled worker in the art that the identification of the genetic defect in a genetic disease, coupled with the provision of the DNA sequences of both normal and disease-causing alleles, provides the full scope of diagnostic and therapeutic aspects of such an invention as can be envisaged using current technology.

### BRIEF DESCRIPTION OF THE DRAWINGS

FIG. 1. Genomic structure of IKBKAP. The figure illustrates the orientation and placement of the 37 exons within a 68 kb genomic region of chromosome 9q31. The primers used for analysis of the splice defect are indicated as 18F (exon 18), 19F (exon 19) and 23R (exon 23). Asterick indicates the locations of the two mutations identified; the mutation associated with the major AJ haplotype is located at bp 6 of intron 20, whereas the mutation association with the minor AJ haplotype is located at bp 73 of exon 19. The 4.8 and 5.9 designations at exon 37 indicate the lengths of the two IKBKAP messages that differ only in the length of their 3′ UTRs.

FIGS. 2A-2C. Demonstration of mutations in IKBKAP. FIG. 2A shows the antisense sequence of the T→C mutation (shown by arrows adjacent to the G and A lanes) at bp 6 of intron 20 that is associated with the major FD haplotype. Lanes 1 and 2 are FD patients homozygous for the major haplotype (homozygous GG), lane 3 is an FD patient heterozygous for the major haplotype and minor haplotype 2 (heterozygous GA), lane 4 is an FD patient heterozygous for the major haplotype and minor haplotype 3 (heterozygous GA), and lanes 5 and 6 are non-FD controls (homozygous AA). FIG. 2B shows heterozygosity for the G→C mutation (shown by arrows adjacent to the G and C lanes) at bp 73 of exon 19. Lane 1 is an FD homozygous for the major haplotype (homozygous GG), lanes 2-4 are three patients heterozygous for the major haplotype and minor haplotype 2 (heterozygous GC), lane 5 is a patient heterozygous for the major haplotype and minor haplotype 3 (homozygous GG), and lane 6 is a non-FD control (homozygous GG). FIG. 2C shows the sequence of the cDNA generated from the RT-PCR of a patient heterozygous for the major and minor 2 haplotypes. The arrow points to the heterozygous G→C mutation in exon 19. The boundary of exons 19 and 20 is also indicated, illustrating that this patient expresses wild-type message that includes exon 20, despite the presence of the major mutation on one allele.

FIGS. 3A-3B. Northern blot analysis of IKBKAP. FIG. 3A is a human multiple tissue northern blot that was hybridized with IKBKAP exon 2 and shows the presence of two messages of 4.8 and 5.9 kb (northern blots hybridized with other IKBKAP probes yielded similar patterns). FIG. 3B is a northern blot generated using rnRNA isolated from lymphoblast cell lines: lanes 1, 2, and 5 FD patients homozygous for the major haplotype; lane 3 individual carrying two definitively non-FD chromosomes, lane 4 FD patient heterozygous for the major haplotype and minor haplotype 2; lane 6 control brain RNA (Clontech). The level of expression of IKBKAP mRNA relative to (3-actin mRNA is quite variable in lymphoblasts. We observed no consistent increase or decrease in mRNA levels between FD patients homozygous for the major haplotype, those heterozygous for the major haplotype and minor haplotype 2, and non-FD individuals.

FIGS. 4A-4B: RT-PCR analysis of the exon 20 region of IKBKAP showing expression of the wild-type message and protein in patients. FIG. 4A was generated using primers 18F (exon 18) and 23R (exon 23). Lanes 1 and 2 are FD patients homozygous for the major haplotype, lane 3 is an FD patient heterozygous for the major haplotype and minor haplotype 2, lanes 4 and 5 are non-FD controls, lane 6 is a water control. FIG. 4B is a western blot generated using cytoplasmic protein isolated from patient lymphoblast cell lines and detected with a carboxyl-terminal antibody. Lanes 2, 4, 6, and 8 are patients homozygous for the major haplotype, lanes 3, 5, 7, and 9 are non-FD controls, lane 1 is a patient heterozygous for the major and minor haplotype 3, and lane 10 is a patient heterozygous for the major and minor haplotype 2 and lane 11 is a Hela cell line sample.

FIG. 5. RT-PCR analysis of the exon 20 region of IKBKAP showing variable expression of the mutant message in FD patients. The analysis was done using primers 19F (exon 19) and 23F (exon 23). Lanes 1 and 2, control fibroblasts; lanes 3, 4, and 5, FD fibroblasts homozygous for the major mutation; lanes 6 and 7 FD lymphoblasts homozygous for the major mutation, lanes 8 and 9 non-FD lymphoblasts, lane 10 FD patient brain stem, lane 11 FD patient temporal lobe (showing a faint 319 bp band and no 393 bp band), lane 12 water control. RT-PCR of control brain RNA (Clontech) showed only the 393 bp band (data not shown).

FIGS. 6-1 to 6-42. The genomic sequence for IKBKAP.

FIGS. 7A-7C—The cDNA sequence for IKBKAP.

FIG. 8—the amino acid sequence of the IKBKAP gene.

FIGS. 9A-9E—Comparison of the amino acid sequence of Ikap across several species. Alignment of the amino acid sequence of Ikap (M_musculus) with that of Homo sapiens (H_sapiens), Drosophila melanogaster (D melanogaster), Saccharomyces cerevisiae (S cervisiae), Arabidopsis thaliana (A_thaliana), and Caenorhabditis elegans (C_elegans).

FIG. 10—Comparison of the Novel Mouse Ikbkap Gene with Multiple Species Homologs.

FIG. 11—Mouse Ikbkap Exon and Introit Boundaries.

FIG. 12—Comparison of the synthetic regions of mouse chromosome 4 (MMU4) and human chromosome 9 (HSA9q31). This diagram on the left shows the location of Ikbkap in relation to mapped and genetic markers (boldface). Distances are given in centimorgans. The positions of the homologous genes that map to human chromosome 9q31 are shown on the right.

### DETAILED DESCRIPTION OF THE INVENTION

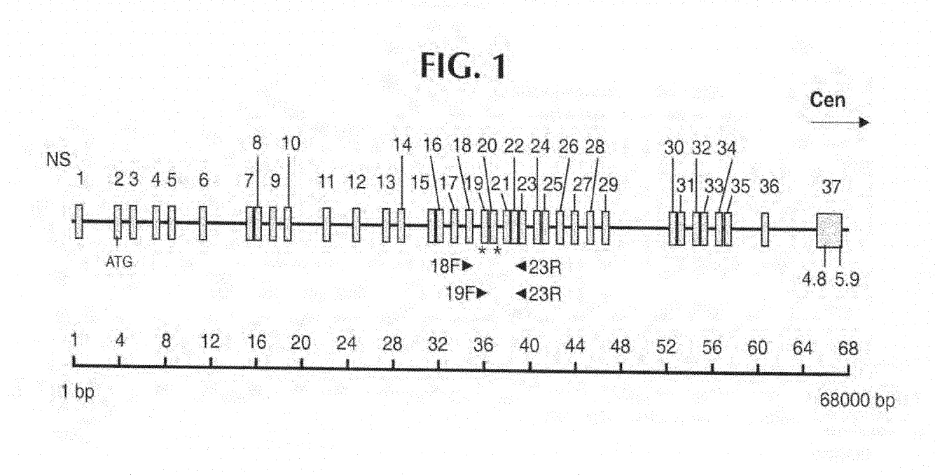

This invention relates to mutations in the IKBKAP gene, which the inventors of the instant application discovered are associated with Familial Dysautonomia. More specifically, the mutation associated with the major haplotype of FD is a T→C change located at bp 6 of intron 20 in the IKBKAP gene as shown in FIG. 1. This mutation can result in skipping of exon 20 in the rnRNA from FD patients, although they continue to express varying levels of wild-type message in a tissue specific manner. The mutation associated with the minor haplotype is a single G→C change at bp 2397 (bp 73 of exon 19) that causes an arginine to proline missense mutation (R696P) that is predicted to disrupt a potential phosphorylation site.

These findings have direct implications for understanding the clinical manifestations of FD, for preventing it and potentially for treating it. The IKAP protein produced from IKBKAP gene was originally isolated as part of a large interleukin-1-inducible IKK complex and described as a regulator of kinases involved in pro-inflammatory cytokine signaling (Cohen et al.

1998). However, a recent report questioned this conclusion, by reporting that cellular IKK complexes do not contain IKAP based on various protein-protein interaction and functional assays. Rather, IKAP appears to be a member of a novel complex containing additional unidentified proteins of 100, 70, 45, and 39 kDa (Krappmann et al. 2000).

IKAP is homologous to the EIp 1 protein of S. cerevisiae, which is encoded by the IKI3 locus and is required for sensitivity to pGKL killer toxin. The human and yeast proteins exhibit 29% identity and 46% similarity over their entire lengths. Yeast Elp 1 protein is part of the RNA polymerase II-associated elongator complex, which also contains Elp2, a WD-40 repeat protein, and Elp3, a histone acetyltransferase (Otero et al. 1999). The human ELP3 gene encodes a 60 kDa histone acetyltransferase that shows more than 75% identity with yeast E1p3 protein, but no 60 kDa protein has been found in the human IKAP-containing protein complex. Consequently, it is considered unlikely that IKAP is a member of a functionally conserved mammalian elongator complex (Krappmann et al. 2000). Instead, it has been reported that the protein may play a role in general gene activation mechanisms, as overexpression of IKAP interferes with the activity of both NF-kB-dependent and independent reporter genes (Krappmann et al. 2000). Therefore, the FD phenotype may be caused by aberrant expression of genes crucial to the development of the sensory and autonomic nervous systems, secondary to the loss of a functional IKAP protein in specific tissues.

FD is unique among Ashkenazi Jewish disorders in that one mutation accounts for >99.5% of the disease chromosomes. As in other autosomal recessive diseases with no phenotype in heterozygous carriers, one might have expected to find several different types of mutations producing complete inactivation of the DYS gene in the AJ population. The fact that the major FD mutation does not produce complete inactivation, but rather allows variable tissue-specific expression of IKAP, may explain this lack of mutational diversity. Mutations causing complete inactivation of IKAP in all tissues might cause a more severe or even lethal phenotype. Indeed, CG10535, the apparent Drosophila melanogaster homologue of IKBKAP, maps coincident with a larval recessive lethal mutation (1(3)04629) supporting the essential nature of the protein (FlyBase). Thus, the array of mutations that can produce the FD phenotype may be limited if they must also allow expression of functional or partially functional IKAP in some tissues to permit survival. With the identification of IKBKAP as DYS, it will now be possible to test this inactivation hypothesis in a mammalian model system.

Despite the overwhelming predominance of a single mutation in FD patients, the disease phenotype is remarkably variable both within and between families. The nature of the major FD mutation makes it tempting to consider that this phenotypic variability might relate to the frequency of exon 20 skipping in specific tissues and at specific developmental stages, which may be governed by variations in many factors involved in RNA splicing. Even a small amount of normal IKAP protein expressed in critical tissues might permit sufficient neuronal survival to alleviate the most severe phenotypes. This possibility is supported by the relatively mild phenotype associated with the presence of the R696P mutation, which is predicted to permit expression of an altered full-length IKAP protein that may retain some functional capacity. To date, this minor FD mutation has only been seen in four patients heterozygous for the major mutation. Consequently, it is uncertain whether homozygotes for the R696P mutation would display any phenotypic abnormality characteristic of FD. The single patient with minor haplotype 3 and mixed ancestry, whose mutation has yet to be found, is also a compound heterozygote with the major haplotype. The existence of minor haplotype 3 indicates that IKBKAP mutations will be found outside the AJ population, but like the R696P mutation, it is difficult to predict the severity of phenotype that would result from homozygosity.

Since FD affects the development and maintenance of the sensory and autonomic nervous systems, the identification of IKBKAP as the DYS gene allows for further investigation of the role of IKAP and associated proteins in the sensory and autonomic nervous systems. Of more immediate practical importance, however, the discovery of the single base mutation that characterizes >99.5% of FD chromosomes will permit efficient, inexpensive carrier testing in the

AJ population, to guide reproductive choices and reduce the incidence of FD. The nature of the major mutation also offers some hope for new approaches to treatment of FD. Despite the presence of this mutation, lymphoblastoid cells from patients are capable of producing full-length wild-type mRNA and normal IKAP protein, while in neuronal tissue exon 20 is skipped, presumably leading to a truncated product. Investigation of the mechanism that permits lymphoblasts to be relatively insensitive to the potential effect of the mutation on splicing may suggest strategies to prevent skipping of exon 20 in other cell types. An effective treatment to prevent the progressive neuronal loss of FD may be one aimed at facilitating the production of wild-type rnRNA from the mutant gene rather than exogenous administration of the missing IKAP protein via gene therapy.

### FD Screening

With knowledge of the primary mutation and secondary mutation of the FD gene as disclosed herein, screening for presymptomatic homozygotes, including prenatal diagnosis, and screening for heterozygous carriers can be readily carried out.

### 1. Nucleic Acid Based Screening

Individuals carrying mutations in the FD gene may be detected at either the DNA or RNA level using a variety of techniques that are well known in the art. The genomic DNA used for the diagnosis may be obtained from an individual's cells, such as those present in peripheral blood, urine, saliva, bucca, surgical specimen, and autopsy specimens. The DNA may be used directly or may be amplified enzymatically in vitro through use of PCR (Saiki et al. Science 239:487-491 (1988)) or other in vitro amplification methods such as the ligase chain reaction (LCR) (Wu and Wallace Genomics 4:560-569 (1989)), strand displacement amplification (SDA) (Walker et al. PNAS USA 89:392-396 (1992)), self-sustained sequence replication (3SR) (Fahy et al. PCR Methods Appl. 1:25-33 (1992)), prior to mutation analysis in situ hybridization may also be used to detect the FD gene.

The methodology for preparing nucleic acids in a form that is suitable for mutation detection is well known in the art. For example, suitable probes for detecting a given mutation include the nucleotide sequence at the mutation site and encompass a sufficient number of nucleotides to provide a means of differentiating a normal from a mutant allele. Any probe or combination of probes capable of detecting any one of the FD mutations herein described are suitable for use in this invention. Examples of suitable probes include those complementary to either the coding or noncoding strand of the DNA. Similarly, suitable PCR primers are complementary to sequences flanking the mutation site. Production of these primers and probes can be carried out in accordance with any one of the many routine methods, e.g., as disclosed in Sambrook et al., and those disclosed in WO 93/06244 for assays for Goucher disease.

Probes for use with this invention should be long enough to specifically identify or amplify the relevant FD mutations with sufficient accuracy to be useful in evaluating the risk of an individual to be a carrier or having the FD disorder. In general, suitable probes and primers will comprise, preferably at a minimum, an oligomer of at least 16 nucleotides in length. Since calculations for mammalian genomes indicate that for an oligonucleotide 16 nucleotides in length, there is only one chance in ten that a typical cDNA library will fortuitously contain a sequence that exactly matches the sequence of the nucleotide. Therefore, suitable probes and primers are preferably 18 nucleotides long, which is the next larger oligonucleotide fully encoding an amino acid sequence (i.e., 6 amino acids in length).

By use of nucleotide and polypeptide sequences provided by this invention, safe, effective and accurate testing procedures are also made available to identify carriers of mutant alleles of IKBKAP, as well as pre- and postnatal diagnosis of fetuses and live born patients carrying either one or two mutant alleles. This affords potential parents the opportunity to make reproductive decisions prior to pregnancy, as well as afterwards, e.g., if chorionic villi sampling or amniocentesis is performed early in pregnancy. Thus, prospective parents who know that they are both carriers may wish to determine if their fetus will have the disease, and may wish to terminate such a pregnancy, or to provide the physician with the opportunity to begin treatment as soon as possible, including prenatally. In the case where such screening has not been performed, and therefore the carrier status of the patient is not known, and where FD disease is part of the differential diagnosis, the present invention also provides a method for making the diagnosis genetically.

Many versions of conventional genetic screening tests are known in the art. Several are disclosed in detail in WO 91/02796 for cystic fibrosis, in U.S. Pat. No. 5,217,865 for Tay-Sachs disease, in U.S. Pat. No. 5,227,292 for neurofibromatosis and in WO 93/06244 for Goucher disease. Thus, in accordance with the state of the art regarding assays for such genetic disorders, several types of assays are conventionally prepared using the nucleotides, polypeptides and antibodies of the present invention. For example: the detection of mutations in specific DNA sequences, such as the FD gene, can be accomplished by a variety of methods including, but not limited to, restriction-fragment-length-polymorphism detection based on allele-specific restriction-endonuclease cleavage (Kan and Dozy Lancet ii :910-912 (1978)), hybridization with allele-specific oligonucleotide probes (Wallace et al. Nucl Acids Res 6:3543-3557 (1978)), including immobilized oligonucleotides (Saiki et al. PNAS USA 86:6230-6234 (1989)) or oligonucleotide arrays (Maskos and Southern Nucl Acids Res 21:2269-2270 (1993)), allele-specific PCR (Newton et al. Nucl Acids Res 17:2503-25 16 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. PNAS USA 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. PNAS USA 85:4397-4401 (1988)) or enzymatic (Youil et al. PNAS USA 92:87-91 (1995)) cleavage of heteroduplex DNA, methods based on allele specific primer extension (Syvanen et al. Genomics 8:684-692 (1990)), genetic bit analysis (GBA) (Nikiforov et al. Nucl Acids Res 22:4167-4175 (1994)), the oligonucleotide-ligation assay (OLA) (Landegren et al. Science 241:1077 (1988)), the allele-specific ligation chain reaction (LCR) (Barrany PNAS USA 88:189-193 (1991)), gap-LCR (Abravaya et al. Nucl Acids Res 23:675-682 (1995)), and radioactive and/or fluorescent DNA sequencing using standard procedures well known in the art.

As will be appreciated, the mutation analysis may also be performed on samples of RNA by reverse transcription into cDNA therefrom. Furthermore, mutations may also be detected at the protein level using, for example, antibodies specific for the mutant and normal FD protein, respectively. It may also be possible to base an FD mutation assay on altered cellular or subcellular localization of the mutant form of the FD protein.

### 2. Antibodies

Antibodies can also be used for the screening of the presence of the FD gene, the mutant FD gene, and the protein products therefrom. In addition, antibodies are useful in a variety of other contexts in accordance with this invention. As will be appreciated, antibodies can be raised against various epitopes of the FD protein. Such antibodies can be utilized for the diagnosis of FD and, in certain applications, targeting of affected tissues.

For example, antibodies can be used to detect truncated FD protein in neuronal cells, the detection of which indicates that an individual possesses a mutation in the IKBKAP gene.

Thus, in accordance with another aspect of the present invention a kit is provided that is suitable for use in screening and assaying for the presence of the FD gene by an immunoassay through use of an antibody which specifically binds to a gene product of the FD gene in combination with a reagent for detecting the binding of the antibody to the gene product.

Antibodies raised in accordance with the invention can also be utilized to provide extensive information on the characteristics of the protein and of the disease process and other valuable information which includes but is not limited to:

1. Antibodies can be used for the immunostaining of cells and tissues to determine the precise localization of the FD protein. Immunofluorescence and immuno-electron microscopy techniques which are well known in the art can be used for this purpose. Defects in the FD gene or in other genes which cause an altered localization of the FD protein are expected to be localizable by this method.

2. Antibodies to distinct isoforms of the FD protein (i.e., wild-type or mutant-specific antibodies) can be raised and used to detect the presence or absence of the wild-type or mutant gene products by immunoblotting (Western blotting) or other immunostaining methods. Such antibodies can also be utilized for therapeutic applications where, for example, binding to a mutant form of the FD protein reduces the consequences of the mutation.

3. Antibodies can also be used as tools for affinity purification of FD protein. Methods such as immunoprecipitation or column chromatography using immobilized antibodies are well known in the art and are further described in Section (II)(B)(3), entitled “Protein Purification” herein.

4. Immunoprecipitation with specific antibodies is useful in characterizing the biochemical properties of the FD protein. Modifications of the FD protein (i.e., phosphorylation, glycosylation, ubiquitization, and the like) can be detected through use of this method. Immunoprecipitation and Western blotting are also useful for the identification of associating molecules that may be involved in the mammalian elongation complex.

5. Antibodies can also be utilized in connection with the isolation and characterization of tissues and cells which express FD protein. For example, FD protein expressing cells can be isolated from peripheral blood, bone marrow, liver, and other tissues, or from cultured cells by fluorescence activated cell sorting (FACS) Harlow et al., eds., Antibodies: A Laboratory Manual, pp. 394-395, Cold Spring Harbor Press, N.Y. (1988). Cells can be mixed with antibodies (primary antibodies) with or without conjugated dyes. If nonconjugated antibodies are used, a second dye-conjugated antibody (secondary antibody) which binds to the primary antibody can be added This process allows the specific staining of cells or tissues which express the FD protein.

Antibodies against the FD protein are prepared by several methods which include, but are not limited to:

1. The potentially immunogenic domains of the protein are predicted from hydropathy and surface probability profiles. Then oligopeptides which span the predicted immunogenic sites are chemically synthesized. These oligopeptides can also be designed to contain the specific mutant amino acids to allow the detection of and discrimination between the mutant versus wild-type gene products. Rabbits or other animals are immunized with the synthesized oligopeptides coupled to a carrier such as KLH to produce anti-FD protein polyclonal antibodies. Alternatively, monoclonal antibodies can be produced against the synthesized oligopeptides using conventional techniques that are well known in the art Harlow et al., eds., Antibodies: A Laboratory Manual, pp. 151-154, Cold Spring Harbor Press, N.Y. (1988). Both in vivo and in vitro immunization techniques can be used. For therapeutic applications, “humanized” monoclonal antibodies having human constant and variable regions are often preferred so as to minimize the immune response of a patient against the antibody. Such antibodies can be generated by immunizing transgenic animals which contain human immunoglobulin genes. See Jakobovits et al. Ann NY Acad Sci 764:525-535 (1995).

2. Antibodies can also be raised against expressed FD protein products from cells. Such expression products can include the full length expression product or parts or fragments thereof. Expression can be accomplished using conventional expression systems, such as bacterial, baculovirus, yeast, mammalian, and other overexpression systems using conventional recombinant DNA techniques. The proteins can be expressed as fusion proteins with a histidine tag, glutathione-S-transferase, or other moieties, or as nonfused proteins. Expressed proteins can be purified using conventional protein purification methods or affinity purification methods that are well known in the art. Purified proteins are used as immunogens to generate polyclonal or monoclonal antibodies using methods similar to those described above for the generation of antipeptide antibodies.

In each of the techniques described above, once hybridoma cell lines are prepared, monoclonal antibodies can be made through conventional techniques of, for example, priming mice with pristane and interperitoneally injecting such mice with the hybrid cells to enable harvesting of the monoclonal antibodies from ascites fluid.

In connection with synthetic and semi-synthetic antibodies, such terms are intended to cover antibody fragments, isotype switched antibodies, humanized antibodies (mouse-human, human-mouse, and the like), hybrids, antibodies having plural specificities, fully synthetic antibody-like molecules, and the like.

### 3. Expression Systems

Expression systems for the FD gene product allow for the study of the function of the FD gene product, in either normal or wild-type form and/or mutated form. Such analyses are useful in providing insight into the disease causing process that is derived from mutations in the gene.

“Expression systems” refer to DNA sequences containing a desired coding sequence and control sequences in operable linkage, so that hosts transformed with these sequences are capable of producing the encoded proteins. In order to effect transformation, the expression system may be included on a vector; however, the relevant DNA may then also be integrated into the host chromosome.

In general terms, the production of a recombinant form of FD gene product typically involves the following:

First a DNA encoding the mature (used here to include all normal and mutant forms of the proteins) protein, the preprotein, or a fusion of the FD protein to an additional sequence cleavable under controlled conditions such as treatment with peptidase to give an active protein, is obtained. If the sequence is uninterrupted by introns it is suitable for expression in any host. If there are introns, expression is obtainable in mammalian or other eukaryotic systems capable of processing them. This sequence should be in excisable and recoverable form. The excised or recovered coding sequence is then placed in operable linkage with suitable control sequences in an expression vector. The construct is used to transform a suitable host, and the transformed host is cultured under selective conditions to effect the production of the recombinant FD protein. Optionally the FD protein is isolated from the medium or from the cells and purified as described in Section entitled “Protein Purification”.

Each of the foregoing steps can be done in a variety of ways. For example, the desired coding sequences can be obtained by preparing suitable cDNA from cellular mRNA and manipulating the cDNA to obtain the complete sequence. Alternatively, genomic fragments may be obtained and used directly in appropriate hosts. The construction of expression vectors operable in a variety of hosts are made using appropriate replicons and control sequences, as set forth below. Suitable restriction sites can, if not normally available, be added to the ends of the coding sequence so as to provide an excisable gene to insert into these vectors.

The control sequences, expression vectors, and transformation methods are dependent on the type of host cell used to express the gene. Generally, prokaryotic, yeast, insect, or mammalian cells are presently useful as hosts. Prokaryotic hosts are in general the most efficient and convenient for the production of recombinant proteins. However, eukaryotic cells, and, in particular, yeast and mammalian cells, are often preferable because of their processing capacity and post-translational processing of human proteins.

Prokaryotes most frequently are represented by various strains of E. coli. However, other microbial strains may also be used, such as Bacillus subtilis and various species of Pseudomonas or other bacterial strains. In such prokaryotic systems, plasmid or bacteriophage vectors which contain origins of replication and control sequences compatible with the host are used. A wide variety of vectors for many prokaryotes are known (Maniatis et al. Molecular Cloning: A Laboratory Manual pp. 1.3-1.11, 2.3-2.125, 3.2-3.48, 2-4.64 (Cold Spring Harbor Laboratory, Cold Spring Harbor, N.Y. (1982)); Sambrook et al. Molecular Cloning: A Laboratory Manual pp. 1-54 (Cold Spring Harbor Laboratory, Cold Spring Harbor, N.Y. (1989)); Meth. Enzymology 68: 357-375 (1979); 101: 307-325 (1983); 152: 673-864 (1987) (Academic Press, Orlando, Fla. Pouwells et al. Cloning Vectors: A Laboratory Manual (Elsevier, Amsterdam (1987))). Commonly used prokaryotic control sequences which are defined herein to include promoters for transcription initiation, optionally with an operator, along with ribosome binding site sequences, include such commonly used promoters as the beta-lactamase (penicillinase) and lactose (lac) promoter systems, the tryptophan (trp) promoter system and the lambda derived PL promoter and N-gene ribosome binding, site, which has become useful as a portable control cassette (U.S. Pat. No. 4,711,845). However, any available promoter system compatible with prokaryotes can be used (Sambrook et al. supra. (1989); Meth. Enzymology supra. (1979, 1983, 1987); John et al . Gene 61: 207-215 (1987).

In addition to bacteria, eukaryotic microbes, such as yeast, may also be used as hosts. Laboratory strain Saccharomyces cerevisiae or Baker's yeast, is most often used although other strains are commonly available.

Vectors employing the 2 micron origin of replication and other plasmid vectors suitable for yeast expression are known (Sambrook et al. supra. (1989); Meth. Enzymology supra. (1979, 1983, 1987); John et al. supra. (1987). Control sequences for yeast vectors include promoters for the synthesis of glycolytic enzymes. Additional promoters known in the art include the promoters for 3-phosphoglycerate kinase, and those for other glycolytic enzymes, such as glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other promoters, which have the additional advantage of transcription controlled by growth conditions, are the promoter regions for alcohol dehydrogenase 2, isocytochrome C, acid phosphatase, degradative enzymes associated with nitrogen metabolism, and enzymes responsible for maltose and galactose utilization. See Sambrook et al. supra. (1989); Meth. Enzymology supra. John et al. supra. (1987). It is also believed that terminator sequences at the 3′ end of the coding sequences are desirable. Such terminators are found in the 3′ untranslated region following the coding sequences in yeast-derived genes. Many of the useful vectors contain control sequences derived from the enolase gene containing plasmid peno46 or the LEU2 gene obtained from Yep13, however, any vector containing a yeast compatible promoter, origin of replication, and other control sequences is suitable (Sambrook et al. supra. (1989); Meth. Enzymology supra. (1979, 1983, 1987); John et al. supra.

It is also, of course, possible to express genes encoding polypeptides in eukaryotic host cell cultures derived from multicellular organisms (Kruse and Patterson Tissue Culture pp. 475-500 (Academic Press, Orlando (1973)); Meth. Enzymology 68: 357-375 (1979); Freshney Culture of Animal Cells; A Manual of Basic Techniques pp. 329-334 (2d ed., Alan R. Liss, N.Y. (1987))). Useful host cell lines include murine myelomas N51, VERO and HeT cells, SF9 or other insect cell lines, and Chinese hamster ovary (CHO) cells. Expression vectors for such cells ordinarily include promoters and control sequences compatible with mammalian cells such as, for example, the commonly used early and later promoters from Simian Virus 40 (SV 40), or other viral promoters such as those from polyoma, adenovirus 2, bovine papilloma virus, or avian sarcoma viruses, herpes virus family (such as cytomegalovirus, herpes simplex virus, or Epstein-Barr virus), or immunoglobulin promoters and heat shock promoters (Sambrook et al. supra. pp. 16.3-16.74 (1989); Meth. Enzymology 152: 684-704 (1987); John et al. supra. In addition, regulated promoters, such as metallothionine (i.e., MT-1 and MT-2), glucocorticoid, or antibiotic gene “switches” can be used.

General aspects of mammalian cell host system transformations have been described by Axel (U.S. Pat. No. 4,399,216). Plant cells are also now available as hosts, and control sequences compatible with plant cells such as the nopaline synthase promoter and polyadenylation signal sequences are available (Pouwells et al. supra. (1987); Meth Enzymology 118: 627-639 (Academic Press, Orlando (1986); Gelvin et al. J. Bact. 172: 1600-1608.

Depending on the host cell used, transformation is done using standard techniques appropriate to such cells (Sambrook et al. supra. pp. 16.30-16.5 (1989); Meth. Enzymology supra 68:357-375 (1979); 101: 307-325 (1983); 152: 673-864 (1987). U.S. Pat. No. 4,399,216; Meth Enzymology supra 118: 627-639 (1986); Gelvin et al. J. Bact. 172: 1600-1608 (1990). Such techniques include, without limitation, calcium treatment employing calcium chloride for prokaryotes or other cells which contain substantial cell wall barriers; infection with Agrobacterium tumefaciens for certain plant cells; calcium phosphate precipitation, DEAE, lipid transfection systems (such as LIPOFECTIN.™. and LIPOFFECTAMINE.™.), and electroporation methods for mammalian cells without cell walls, and, microprojectile bombardment for many cells including, plant cells. In addition, DNA may be delivered by viral delivery systems such as retroviruses or the herpes family, adenoviruses, baculoviruses, or semliki forest virus, as appropriate for the species of cell line chosen.

### C. THERAPEUTICS

Identification of the FD gene and its gene product also has therapeutic implications. Indeed, one of the major aims of this invention is the development of therapies to circumvent or overcome the defect leading to FD disease. Envisioned are pharmacological, protein replacement, antibody therapy, and gene therapy approaches. In addition the development of animal models useful for developing therapies and for understanding the molecular mechanisms of FD disease are envisioned.

### 1. Pharmacological

In the pharmacological approach, drugs which circumvent or overcome the defective FD gene function are sought. In this approach, modulation of FD gene function can be accomplished by agents or drugs which are designed to interact with different aspects of the FD protein structure or function.

Efficacy of a drug or agent, can be identified in a screening program in which modulation is monitored in vitro cell systems. Indeed, the present invention provides for host cell systems which express various mutant FD proteins (especially the T→C and G→C mutations noted in this application) and are suited for use as primary screening systems.

In vivo testing of FD disease-modifying compounds is also required as a confirmation of activity observed in the in vitro assays. Animal models of FD disease are envisioned and discussed in the section entitled “Animal Models”, below, in the present application.

Drugs can be designed to modulate FD gene and FD protein activity from knowledge of the structure and function correlations of FD protein and from knowledge of the specific defect in various FD mutant proteins. For this, rational drug design by use of X-ray crystallography, computer-aided molecular modeling (CAMM), quantitative or qualitative structure-activity relationship (QSAR), and similar technologies can further focus drug discovery efforts. Rational design allows prediction of protein or synthetic structures which can interact with and modify the FD protein activity. Such structures may be synthesized chemically or expressed in biological systems. This approach has been reviewed in Capsey et al., Genetically Engineered Human Therapeutic Drugs, Stockton Press, New York (1988). Further, combinatorial libraries can be designed, synthesized and used in screening programs.

The present invention also envisions that the treatment of FD disease can take the form of modulation of another protein or step in the pathway in which the FD gene or its protein product participates in order to correct the physiological abnormality.

In order to administer therapeutic agents based on, or derived from, the present invention, it will be appreciated that suitable carriers, excipients, and other agents may be incorporated into the formulations to provide improved transfer, delivery, tolerance, and the like.

A multitude of appropriate formulations can be found in the formulary known to all pharmaceutical chemists: Remington's Pharmaceutical Sciences, (15th Edition, Mack Publishing Company, Easton, Pa. (1975)), particularly Chapter 87, by Blaug, Seymour, therein. These formulations include for example, powders, pastes, ointments, jelly, waxes, oils, lipids, anhydrous absorption bases, oil-in-water or water-in-oil emulsions, emulsions carbowax (polyethylene glycols of a variety of molecular weights), semi-solid gels, and semi-solid mixtures containing carbowax.

Any of the foregoing formulations may be appropriate in treatments and therapies in accordance with the present invention, provided that the active agent in the formulation is not inactivated by the formulation and the formulation is physiologically compatible.

### 2. Protein Replacement Therapy

The present invention also relates to the use of polypeptide or protein replacement therapy for those individuals determined to have a defective FD gene. Treatment of FD disease could be performed by replacing the defective FD protein with normal protein or its functional equivalent in therapeutic amounts.

FD polypeptide can be prepared for therapy by any of several conventional procedures. First, FD protein can be produced by cloning the FD cDNA into an appropriate expression vector, expressing the FD gene product from this vector in an in vitro expression system (cell-free or cell-based) and isolating the FD protein from the medium or cells of the expression system. General expression vectors and systems are well known in the art. In addition, the invention envisions the potential need to express a stable form of the FD protein in order to obtain high yields and obtain a form readily amenable to intravenous administration. Stable high yield expression of proteins have been achieved through systems utilizing lipid-linked forms of proteins as described in Wettstein et al. J Exp Med 174:219-228 (1991) and Lin et al. Science 249:677-679 (1990).

FD protein can be prepared synthetically. Alternatively, the FD protein can be prepared from total protein samples by affinity chromatography. Sources would include tissues expressing normal FD protein, in vitro systems (outlined above), or synthetic materials. The affinity matrix would consist of antibodies (polyclonal or monoclonal) coupled to an inert matrix. In addition, various ligands which specifically interact with the FD protein could be immobilized on an inert matrix. General methods for preparation and use of affinity matrices are well known in the art.

Protein replacement therapy requires that FD protein be administered in an appropriate formulation. The FD protein can be formulated in conventional ways standard to the art for the administration of protein substances. Delivery may require packaging in lipid-containing vesicles (such as LIPOFECTIN™ or other cationic or anionic lipid or certain surfactant proteins) that facilitate incorporation into the cell membrane. The FD protein formulations can be delivered to affected tissues by different methods depending on the affected tissue.

### 3. Gene Therapy

Gene therapy utilizing recombinant DNA technology to deliver the normal form, of the FD gene into patient cells or vectors which will supply the patient with gene product in vivo is also contemplated within the scope of the present invention. In gene therapy of FD disease, a normal version of the FD gene is delivered to affected tissue(s) in a form and amount such that the correct gene is expressed and will prepare sufficient quantities of FD protein to reverse the effects of the mutated FD gene. Current approaches to gene therapy include viral vectors, cell-based delivery systems and delivery agents. Further, ex vivo gene therapy could also be useful. In ex vivo gene therapy, cells (either autologous or otherwise) are transfected with the normal FD gene or a portion thereof and implanted or otherwise delivered into the patient. Such cells thereafter express the normal FD gene product in vivo and would be expected to assist a patient with FD disease in avoiding iron overload normally associated with FD disease. Ex vivo gene therapy is described in U.S. Pat. No. 5,399,346 to Anderson et al., the disclosure of which is hereby incorporated by reference in its entirety. Approaches to gene therapy are discussed below:

a. Viral Vectors

Retroviruses are often considered the preferred vector for somatic gene therapy. They provide high efficiency infection, stable integration and stable expression (Friedman, T. Progress Toward Human Gene Therapy. Science 244:1275 (1989)). The full length FD gene cDNA can be cloned into a retroviral vector driven by its endogenous promoter or from the retroviral LTR. Delivery of the virus could be accomplished by direct implantation of virus directly into the affected tissue.

Other delivery systems which can be utilized include adenovirus, adeno-associated virus (AAV), vaccinia virus, bovine papilloma virus or members of the herpes virus group such as Epstein-Barr virus. Viruses can be, and preferably are, replication deficient.

b. Non-Viral Gene Transfer

Other methods of inserting the FD gene into the appropriate tissues may also be productive. Many of these agents, however, are of lower efficiency than viral vectors and would potentially require infection in vitro, selection of transfectants, and reimplantation. This would include calcium phosphate, DEAE dextran, electroporation, and protoplast fusion. A particularly attractive idea is the use of liposomes (i.e., LIPOFECTIN™), which might be possible to carry out in vivo. Synthetic cationic lipids and DNA conjugates also appear to show some promise and may increase the efficiency and ease of carrying out this approach.

### 4. Animal Models

The generation of a mouse or other animal model of FD disease is important for both an understanding the biology of the disease but also for testing of potential therapies.

The present invention envisions the creation of an animal model of FD disease by introduction of the FD disease causing mutations in a number of species including mice, rats, pigs, and primates.

Techniques for specifically inactivating or mutating genes by homologous recombination in embryonic stem cells (ES cells) have been described (Capecci Science 244:1288 (1989)). Animals with the inactivated homologous FD gene can then be used to introduce the mutant or normal human FD gene or for introduction of the homologous gene to that species and containing the T→C, G→C or other FD disease-causing mutations. Methods for these transgenic procedures are well known to those versed in the art and have been described by Murphy and Carter, Curr. Opin. Cell Biol. 4:273-279 (1992).

### ILLUSTRATIVE EXAMPLES

The following examples are provided to illustrate certain aspects of the present invention and not intended as limiting the subject matter thereof.

### Example 1

Identification of the IKBKAP gene and the mutations associated with FD were obtained as follows:

### Patient Samples

Blood samples were collected from two major sources, the Dysautonomia Diagnostic and Treatment Center at New York University Medical Center and the Israeli Center for Familial Dysautonomia at Hadassah University Hospital, with approval from the institutional review boards at these institutions, Massachusetts General Hospital and Harvard Medical School. Either F.A. or C.M. diagnosed all patients using established criteria. Epstein Barr virus transformed lymphoblast lines using standard conditions. Fibroblast cell lines were obtained from the Coriell Cell Repositories, Camden, N.J. RNA isolated from post-mortem FD brain was obtained from the Dysautonomia Diagnostic and Treatment Center at NYU. Genomic DNA, total RNA, and mnRNA were prepared using commercial kits (Invitrogen and Molecular Research Center, Inc). Cytoplasmic protein was extracted from lymphoblasts as previously described (Krappmann et al. 2000).

### Identification of IKBKAP and Mutation Analysis

Exon trapping experiments of cosmids from a physical map of the candidate region yielded 5 exons that were used to screen a human frontal cortex cDNA library. Several cDNA clones were isolated and assembled into a novel transcript encoding a 1332 AA protein that was later identified as IKBKAP (Cohen et al. 1998). The complete 5.9 kb cDNA sequence of IKBKAP has been submitted to GenBank under accession number AF153419. In order to screen for mutations in FD patients, total lymphoblast RNA was reverse transcribed and overlapping sections of IKBKAP were amplified by PCR and sequenced. Evaluation of the splicing defect was performed using the following primers: 18F: GCCAGTGTTTTTGCCTGAG; 19F: CGGATTGTCACTGTTGTGC; 23R: GACTGCTCTCATAGCATCGC (FIG. 1).

### DNA Sequencing

Sequencing was performed using the AmpliCycle sequencing kit (Applied Biosystems) or on an ABI 377 automated DNA sequencer rising the BigDye terminator cycle sequencing kit (Applied Biosystems). The control sequence of the candidate region was obtained by constructing subclone libraries from BACs and sequencing using vector specific primers. The FD sequence was generated by sequencing cosmids from a patient homozygous for the major FD haplotype using sequence specific primers.

### Expression Studies

Several human multiple tissue northern blots (Clontech) were hybridized using the following radioactively labeled probes: IKBKAP exon 2, IKBKAP exons 18/19/20, IKBKAP exon 23, and a 400 bp fragment of the IKBKAP 3′UTR immediately following the stop codon. Poly (A)⁺ RNA was isolated from patient and control lymphoblast lines, northern blotted, and hybridized using a probe representing the full coding sequence of IKBKAP. Cytoplasmic protein extracted from lymphoblast cell lines was western blotted and detected using ECL (Amersharn) with an antibody raised against a peptide comprising the extreme carboxyl terminus (AA 1313-1332) of human IKAP, the protein encoded by IKBKAP (Krappmann et al. 2000).

To identify DYS, exon trapping and cDNA selection were used to clone and characterize all of the genes in the 471 kb candidate region: EPB41L8 (unpublished data) or EHM2 (Shimizu et al. 2000), C9ORF4 (Chadwick et al. 1999a), C9ORF5 (Chadwick et al. 2000), CTNNAL1 (Zhang et al. 1998), a novel gene with homology to the glycine cleavage system H proteins (CG-8) (unpublished data), IKBKAP (Cohen et al. 1998), and ACTL7A and ACTL7B (Chadwick et al. 1999b). As FD is a recessive disorder, the apriori expectation for the mutation was inactivation of one of these genes. Consequently, each of these were screened for mutations by RT-PCR of patient lymphoblast RNA and direct sequencing of all coding regions. Although many SNPs were identified, there was no evidence for a homozygous inactivating mutation. Thus, it was concluded that the mutation would be found in non-coding sequence and the control genomic sequence of the entire 471 kb candidate region was generated using BACs from a physical map. Direct sequence prediction using GENSCAN and comprehensive searches of the public databases did not reveal any additional genes in the candidate region beyond those found by cloning methods. However, SNPs identified during sequence analysis enabled us to refine the haplotype analysis and narrow the candidate interval to 177 kb shared by the major haplotype and the previously described minor haplotype 1 (Blumenfeld et al. 1999). This reduced interval contains 5 genes, CTNNALI, CG-8, IKBKAP, ACTL7A and ACTL7B, all previously screened by RT-PCR without yielding a coding sequence mutation. A cosmid library was constructed from a patient homozygous for the major haplotype, assembled the minimal coverage contig for the now reduced candidate interval, and generated the sequence of the mutant chromosome.

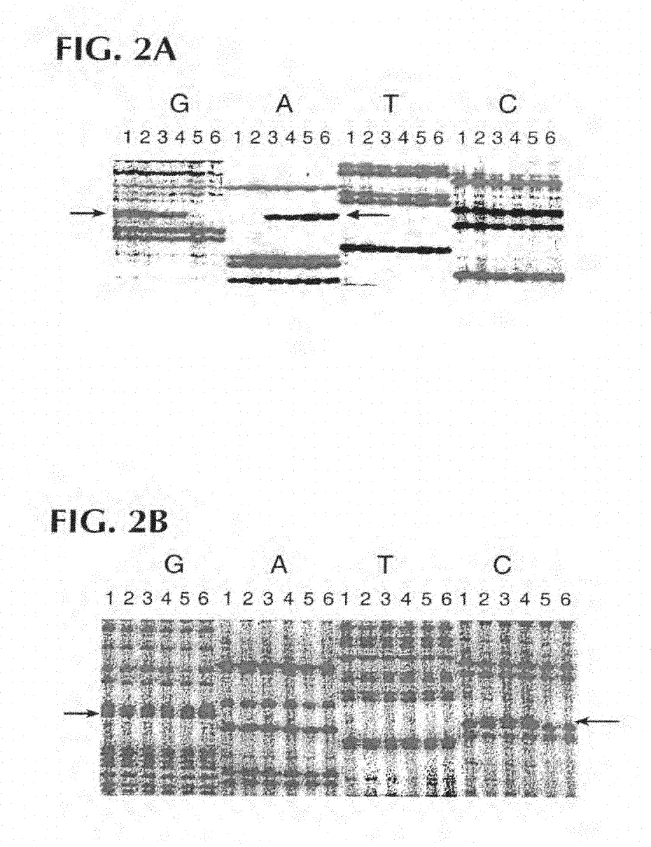

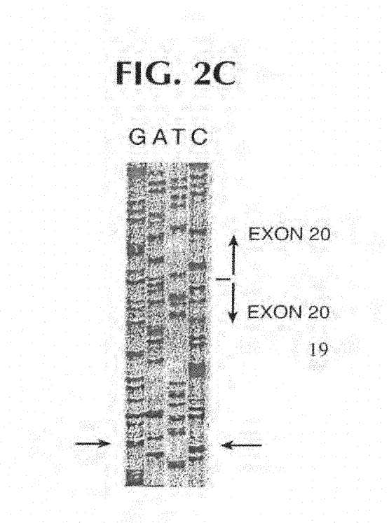

Comparison of the FD and control sequences revealed 152 differences (excluding simple sequence repeat markers), which include 26 variations in the length of dTₙ tracts, 1 VNTR, and 125 base pair changes. Each of the 125 base pair changes was tested in a panel of 50 individuals known to carry two non-FD chromosomes by segregation in FD families. Of the 125 changes tested, only 1 was unique to patients carrying the major FD haplotype. This T→C change is located at bp 6 of intron 20 in the IKBKAP gene depicted in FIG. 1, and is demonstrated in FIG. 2A. IKAP was originally identified as an IkB kinase (IKK) complex-associated protein that can bind both NF-kB inducing kinase (NIK) and IKKs through separate domains and assemble them into an active kinase complex (Cohen et al. 1998). Recent work, however, has shown that IKAP is not associated with IKKs and plays no specific role in cytokine-induced NF-kB signaling (Krappmann et al. 2000). Rather, IKAP was shown to be part of a novel multi-protein complex hypothesized to play a role in general transcriptional regulation.

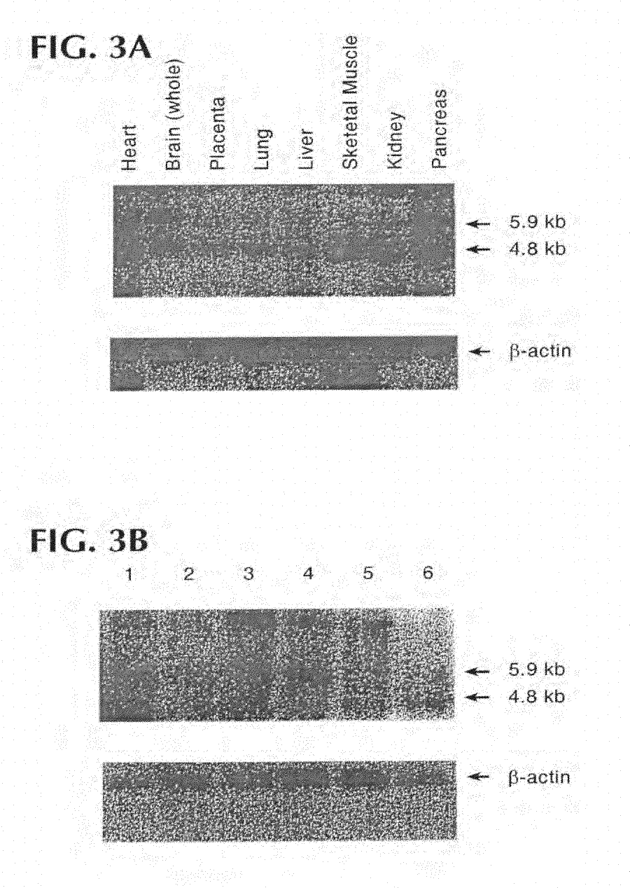

The IKBKAP gene contains 37 exons and encodes a 1332 amino acid protein. The full-length 5.9 kb cDNA (GenBank accession number AF153419) covers 68 kb of genomic sequence, with the start methionine encoded in exon 2. IKBKAP was previously assigned to chromosome 9q34 (GenBank accession number AF044195), but it clearly maps within the FD candidate region of 9₈31. Northern analysis of IKBKAP revealed two mRNAs of 4.8 and 5.9 kb (FIG. 3a and b). The wild-type 4.8 kb mRNA has been reported previously (Cohen et al. 1998), while the second 5.9 kb message differs only in the length of the 3′ UTR and is predicted to encode an identical 150 kDa protein. As seen in FIG. 3B, the putative FD mutation does not eliminate expression of the IKBKAP mRNA in patient lymphoblasts.

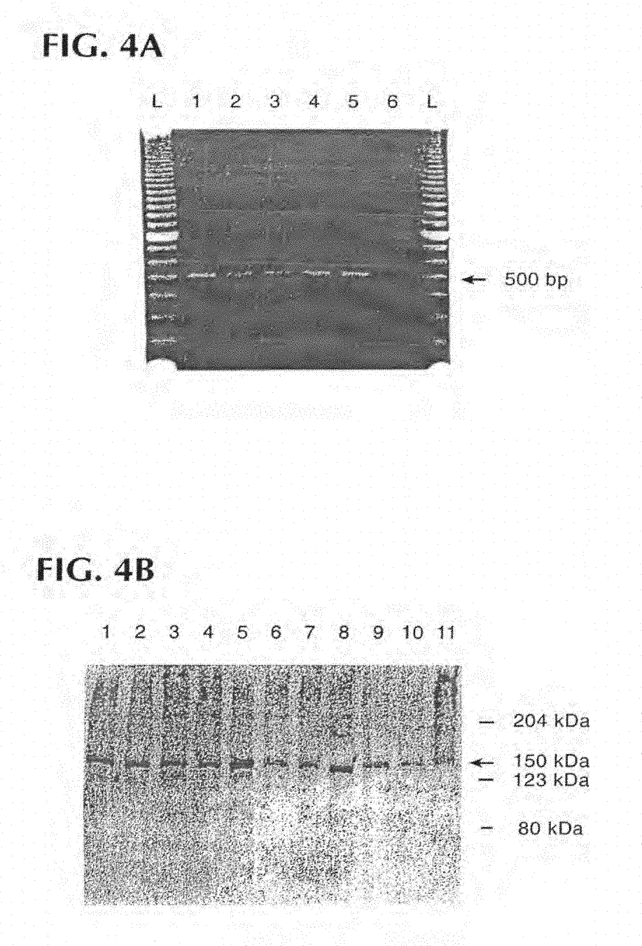

A base pair change at position 6 of the splice donor site might be expected to result in skipping of exon 20 (74 bp), causing a frameshift and therefore producing a truncated protein. However, initial inspection of our RT-PCR experiments in patient lymphoblast RNA using primers located in exons 18 and 23 (FIG. 1) showed a normal length 500 bp fragment that contained exon 20 (FIG. 4A), indicating that patient lymphoblasts express normal IKBKAP message. The Western blot shown in FIG. 4B demonstrates that full-length IKAP protein is expressed in these patient lymphoblasts. However, as the antibody used was directed against the carboxyl-terminus of IKAP it would not be expected to detect any truncated protein should it be present. The presence of apparently normal IKAP in patient cells is at odds with the expectation of an inactivating mutation in this recessive disease.

In the absence of any evidence for a functional consequence of the intron 20 sequence change, the only alteration unique to FD chromosomes, additional genetic evidence was sought to support the view that it represents the FD mutation. The 658 FD chromosomes that carry the major haplotype all show the T→C change. In total, 887 chromosomes have been tested that are definitively non-FD due to their failure to cause the disorder when present in individuals heterozygous for the major FD haplotype. None of these non-FD chromosomes exhibits the T→C mutation, strongly indicating that it is not a rare polymorphism. The frequency of the mutation in random AJ chromosomes was 14/1012 (gene frequency 1/72; carrier frequency 1/36), close to the expected carrier frequency of 1/32 (Maayan et al. 1987).

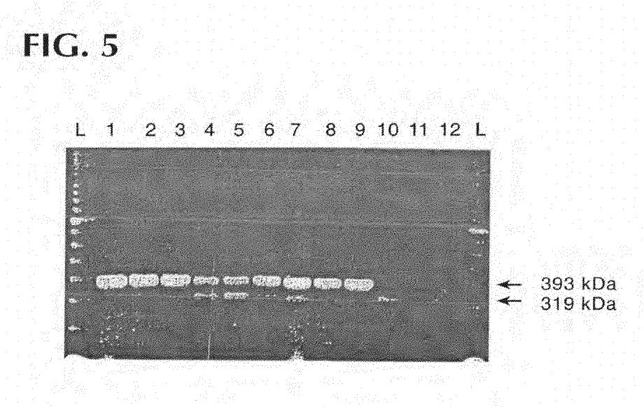

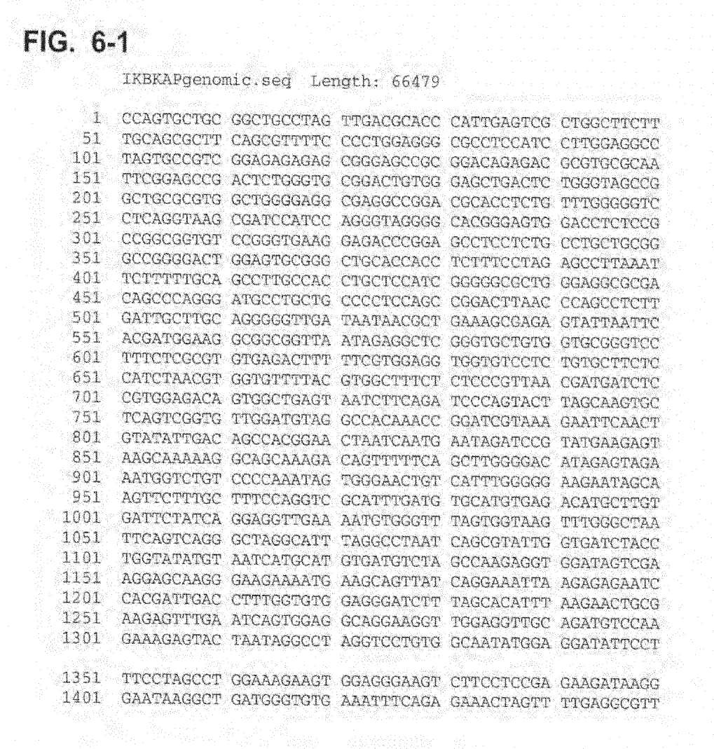

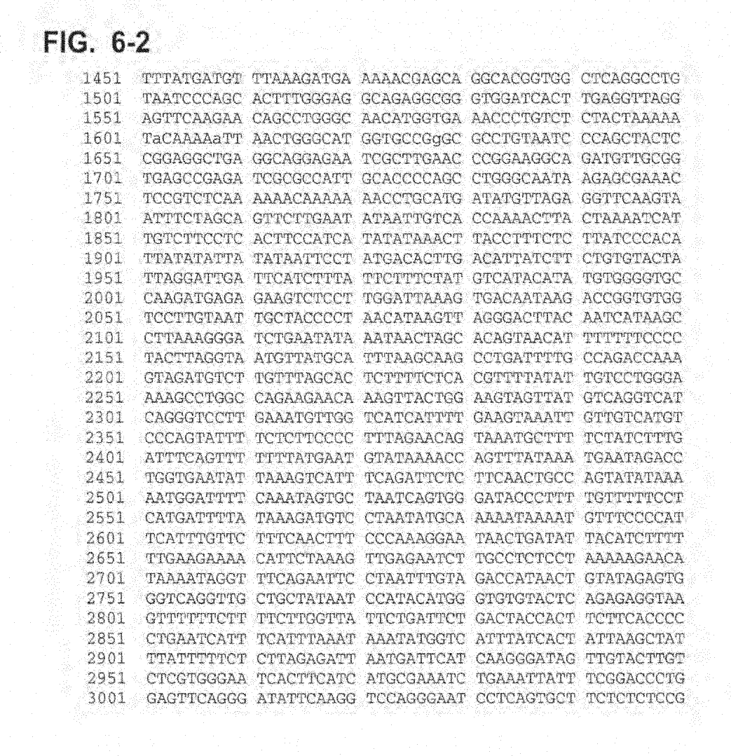

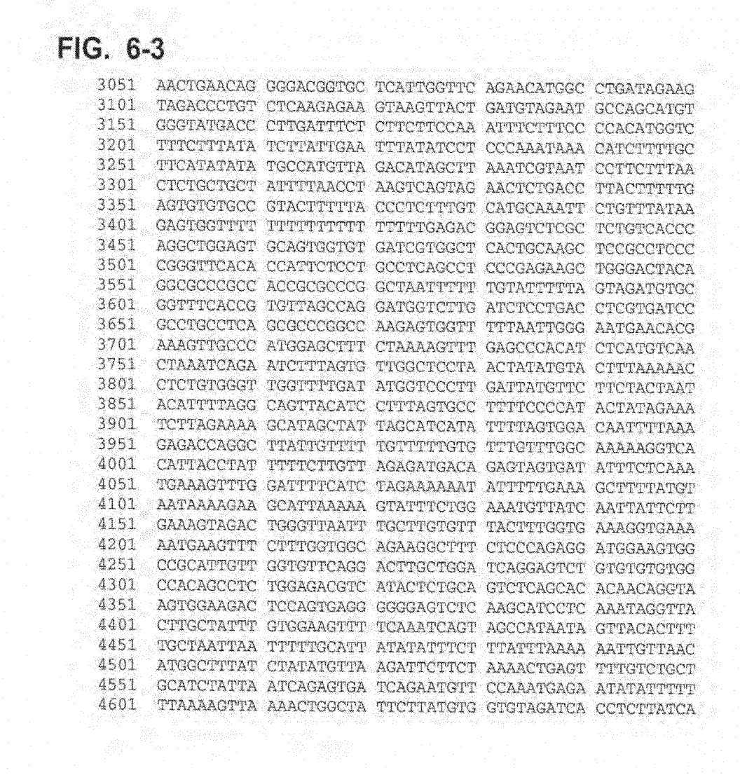

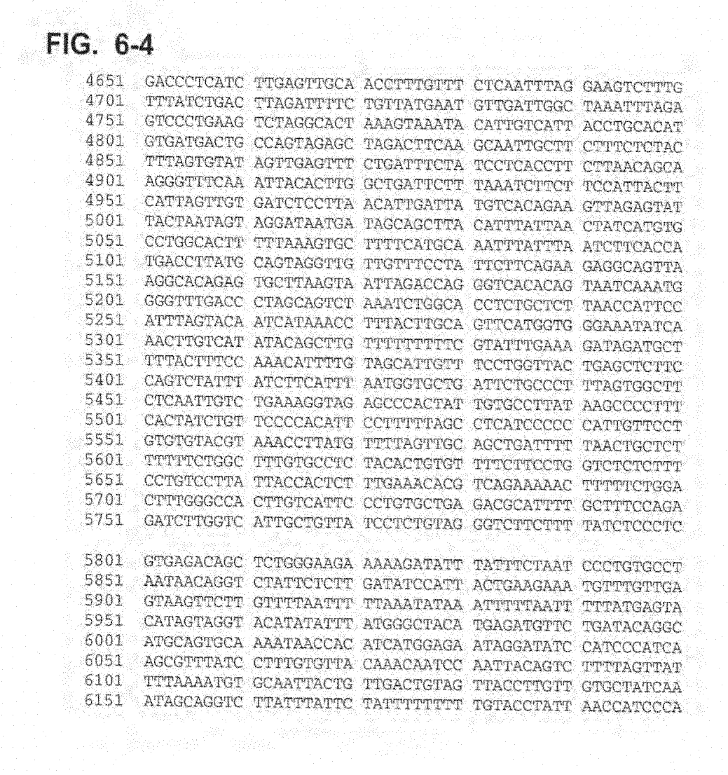

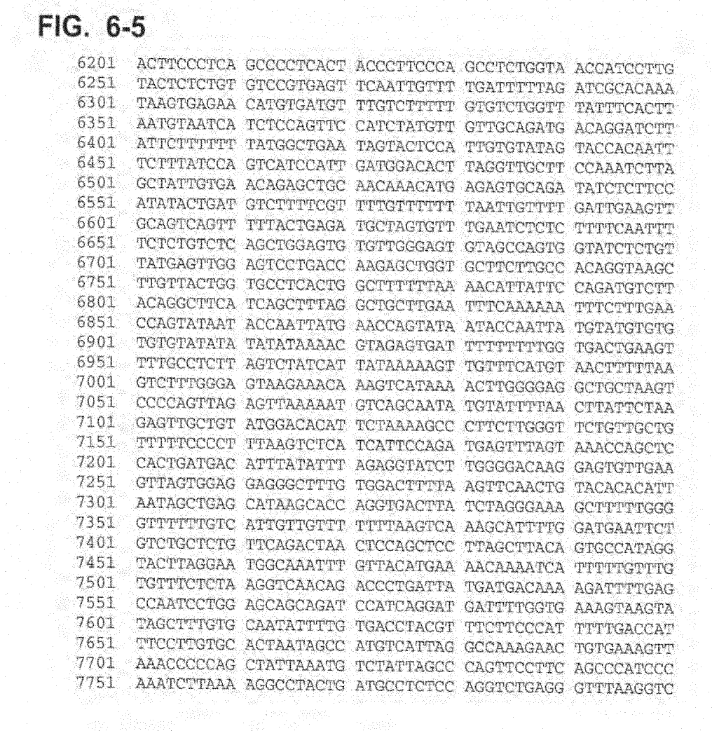

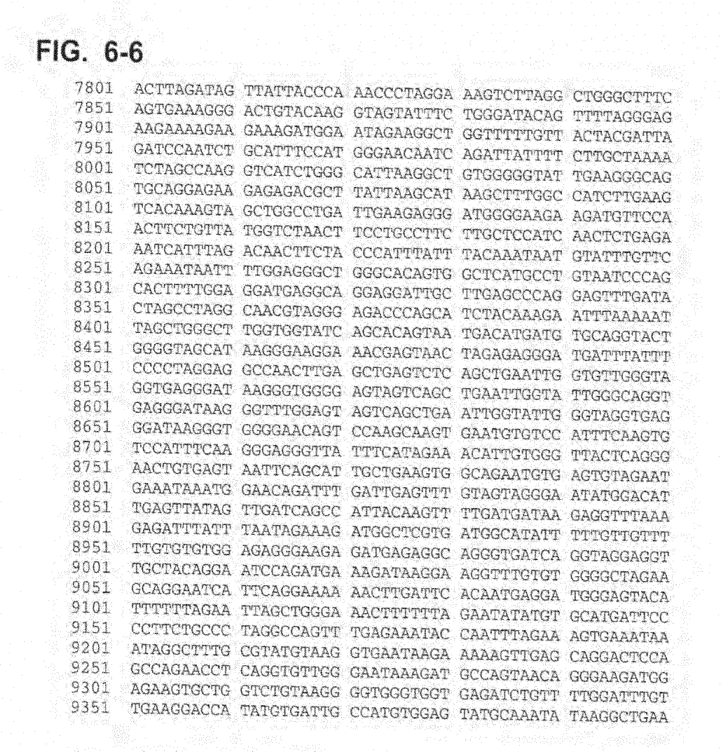

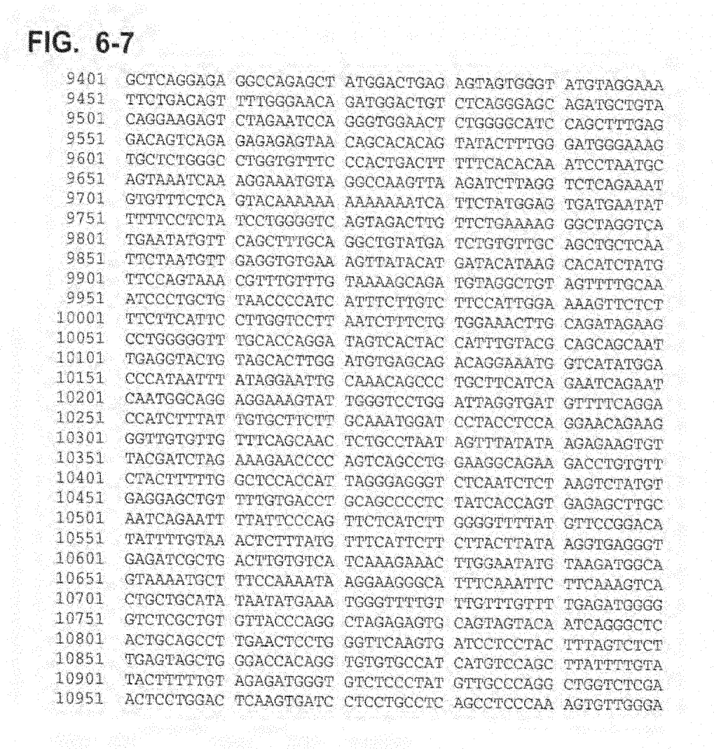

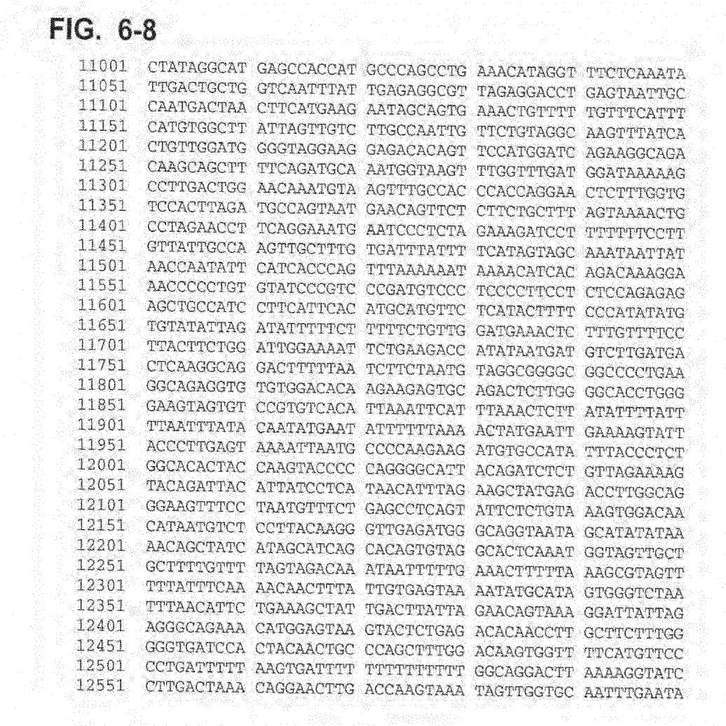

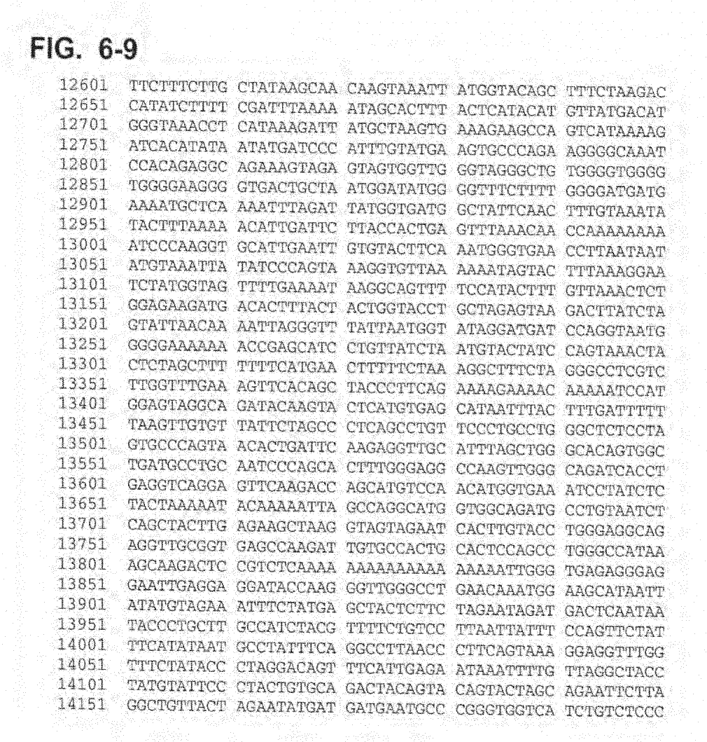

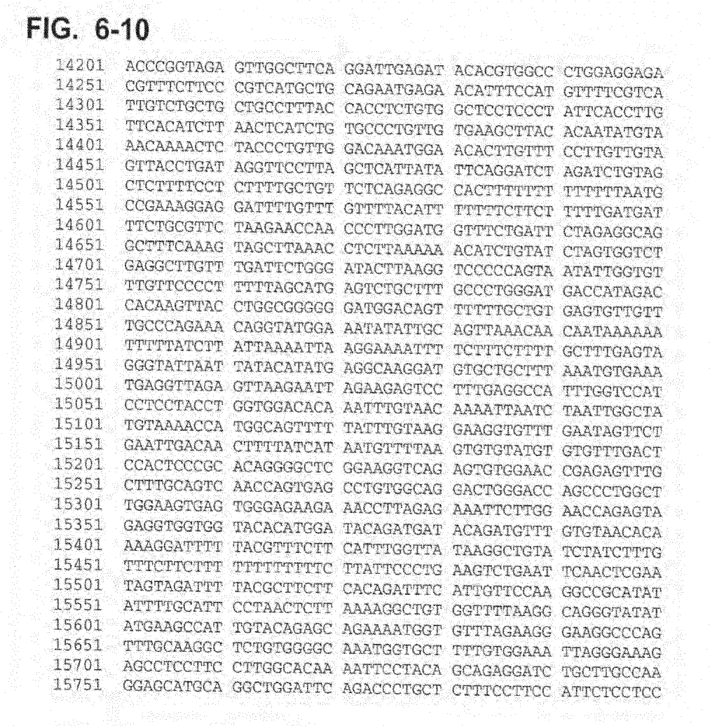

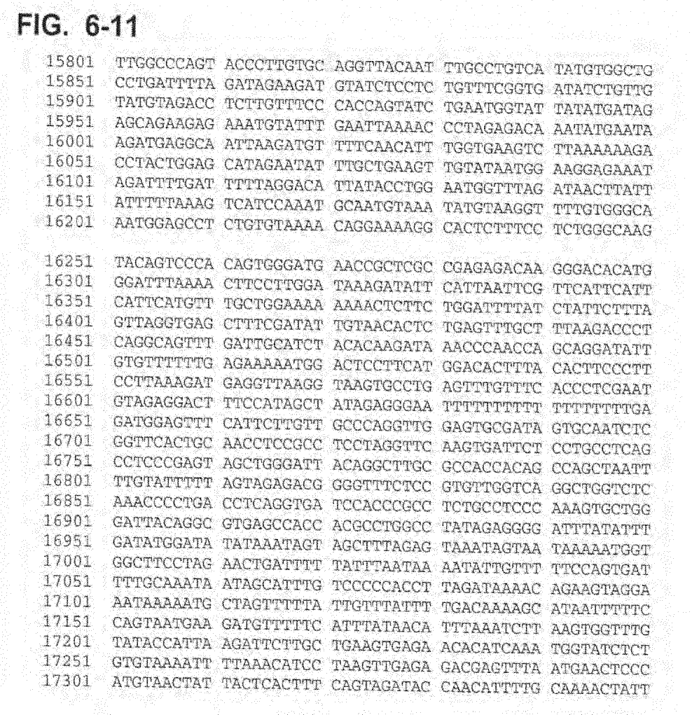

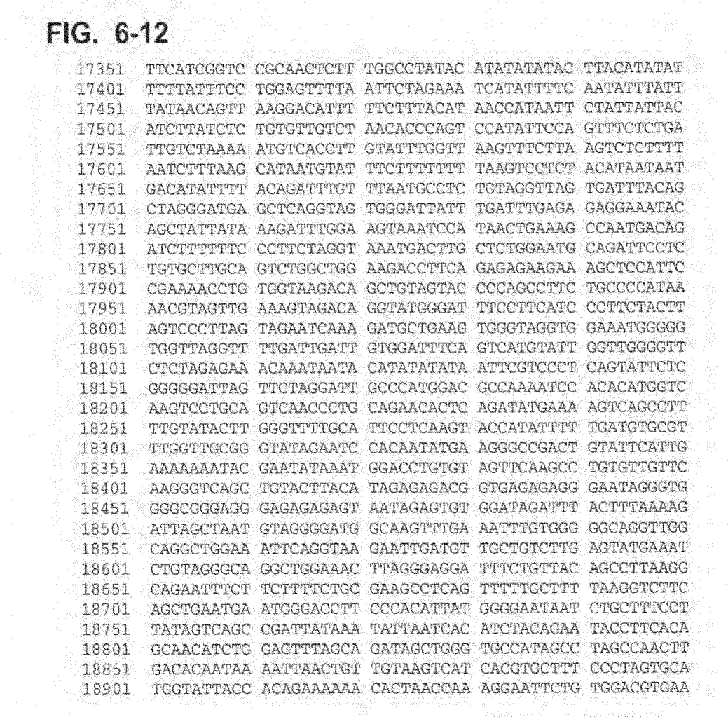

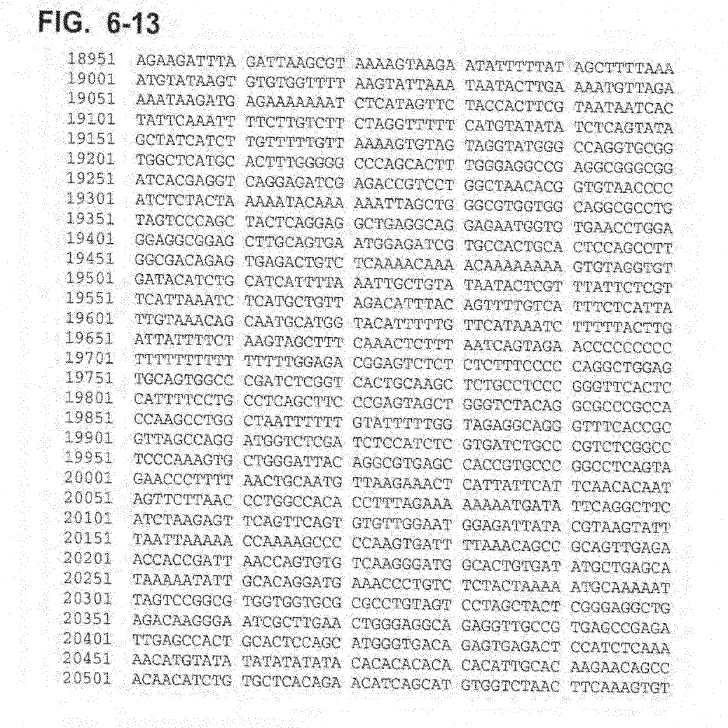

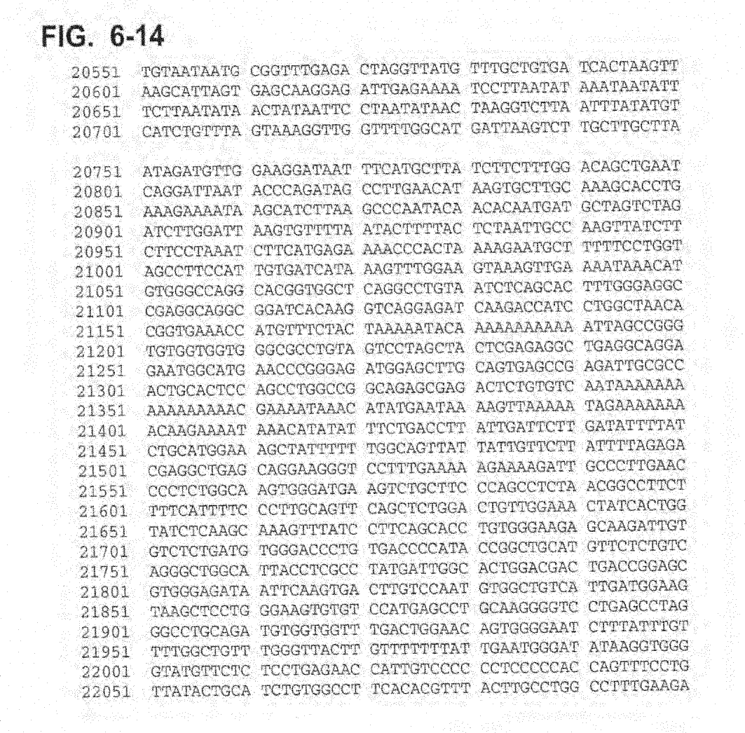

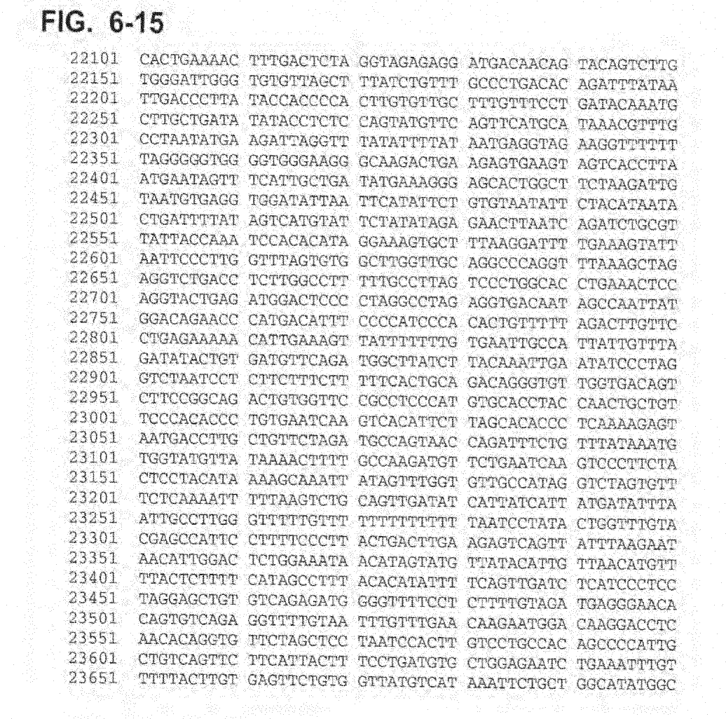

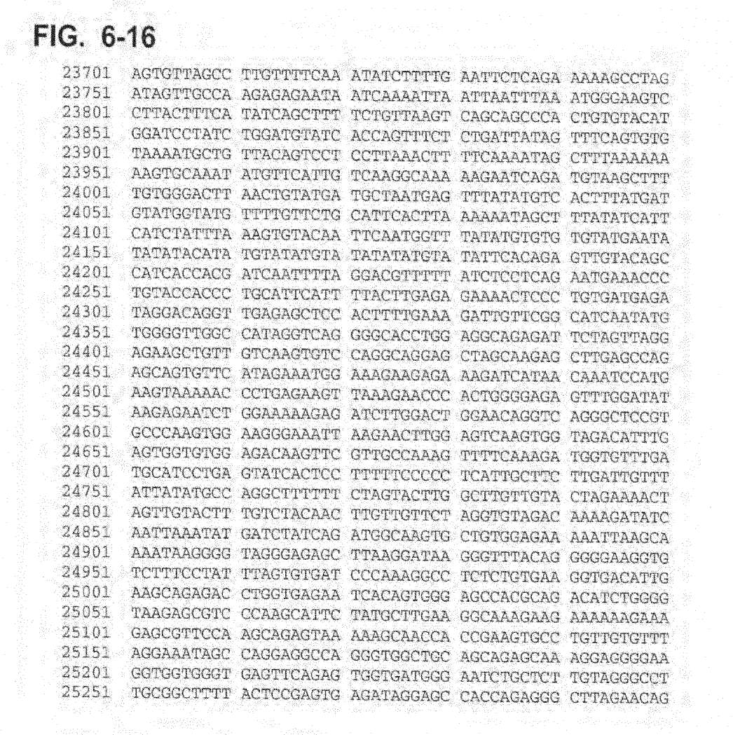

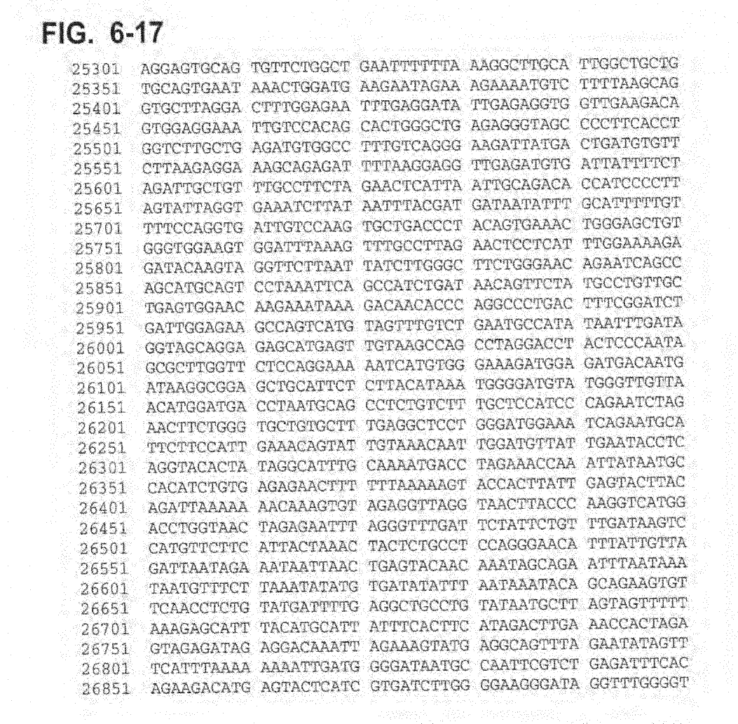

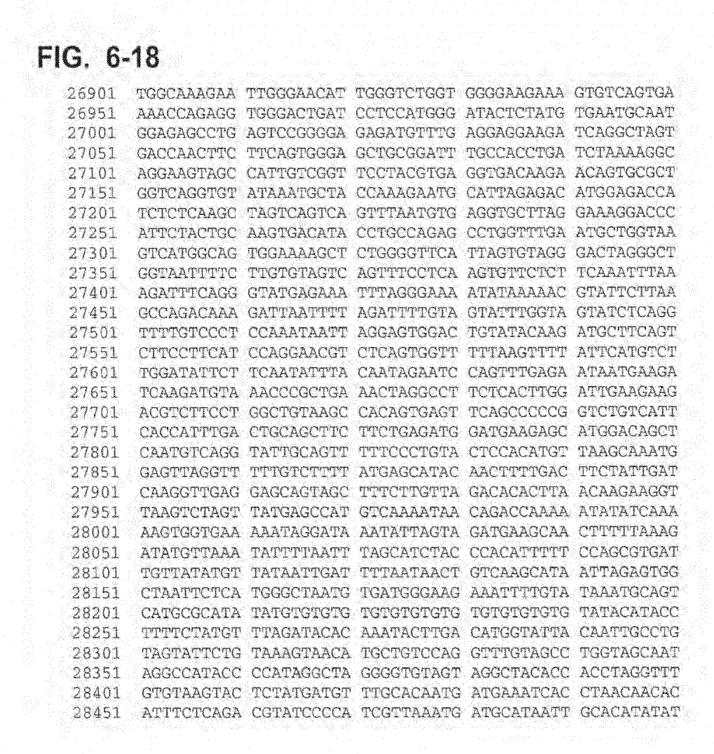

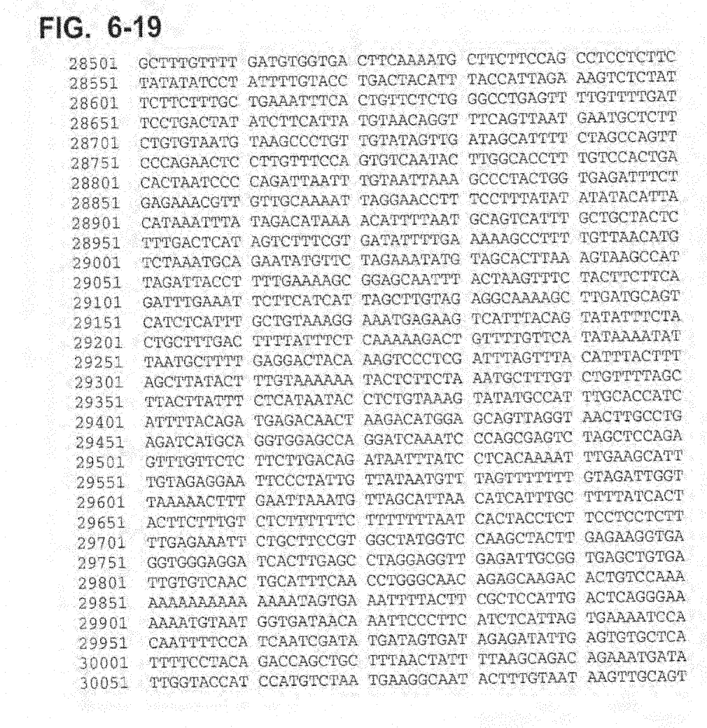

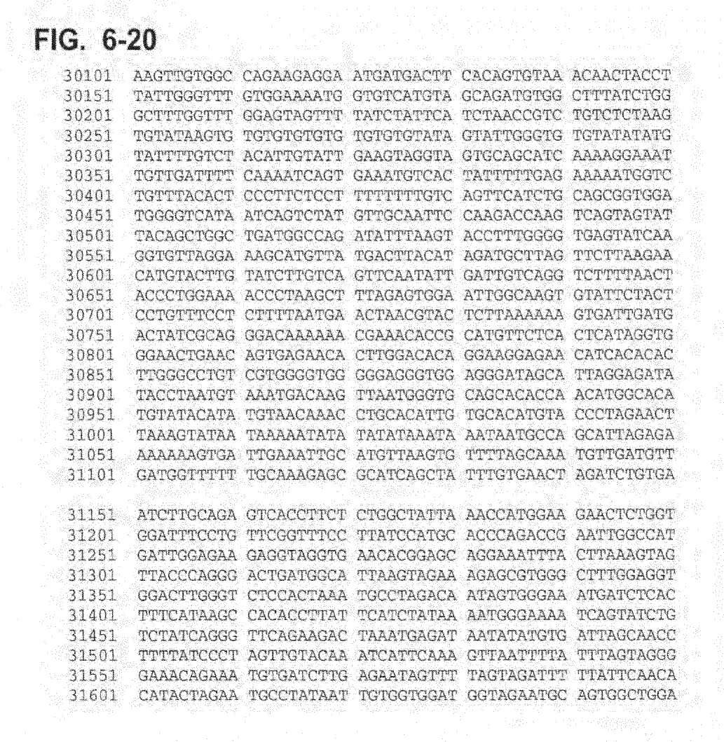

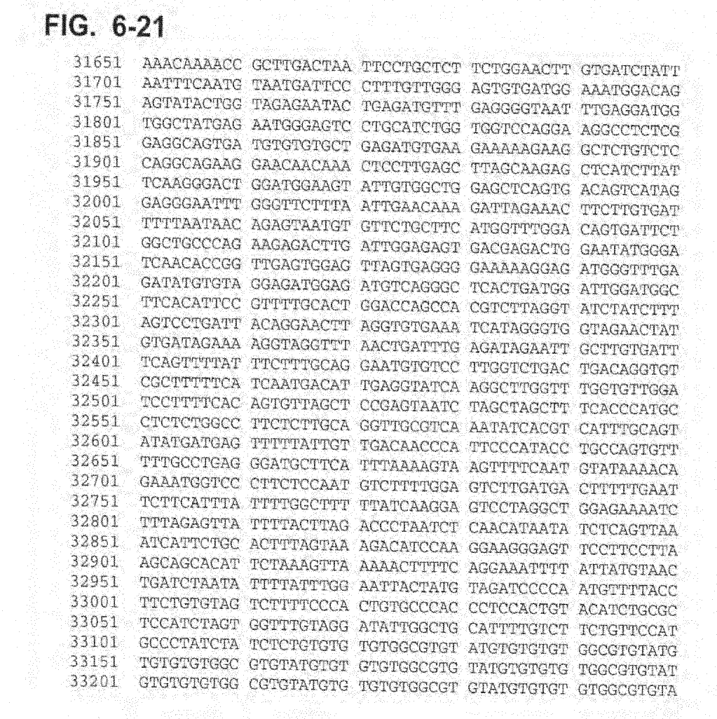

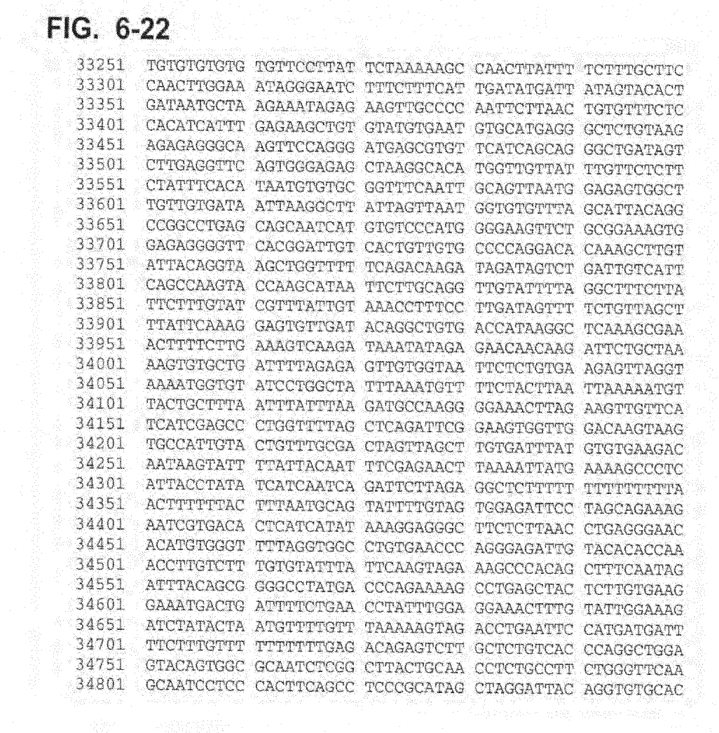

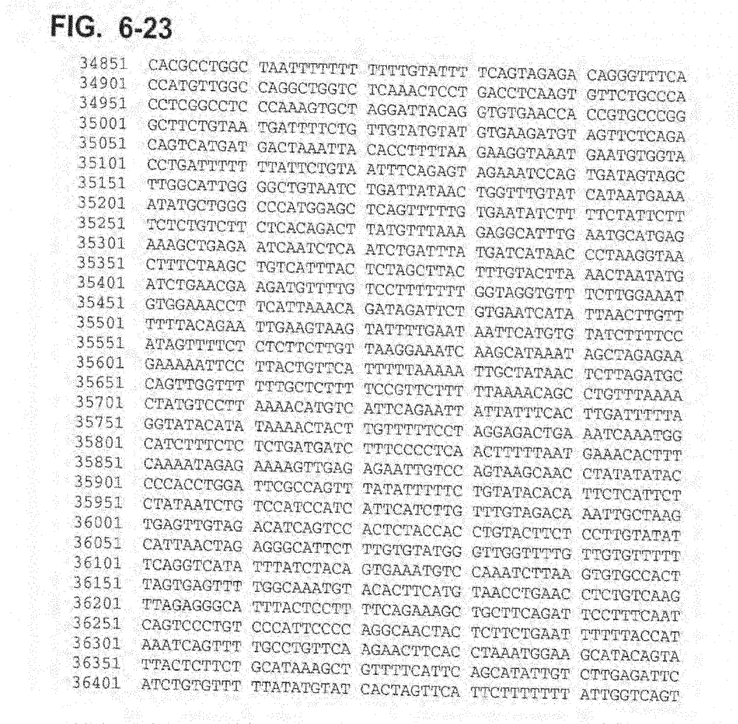

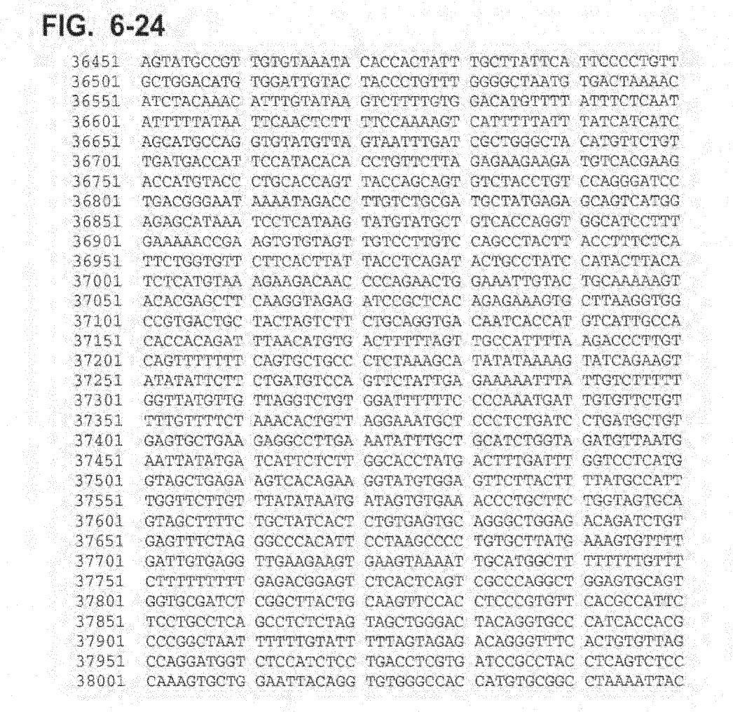

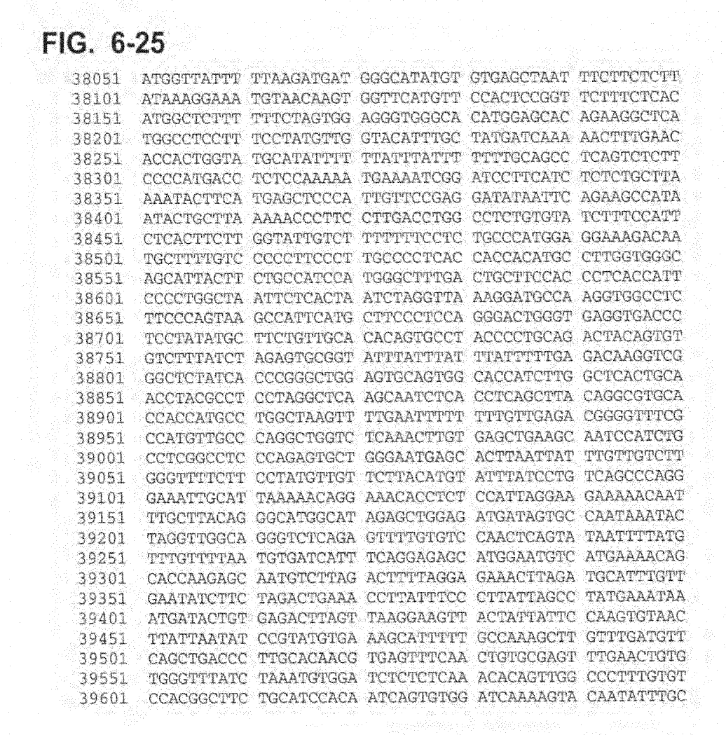

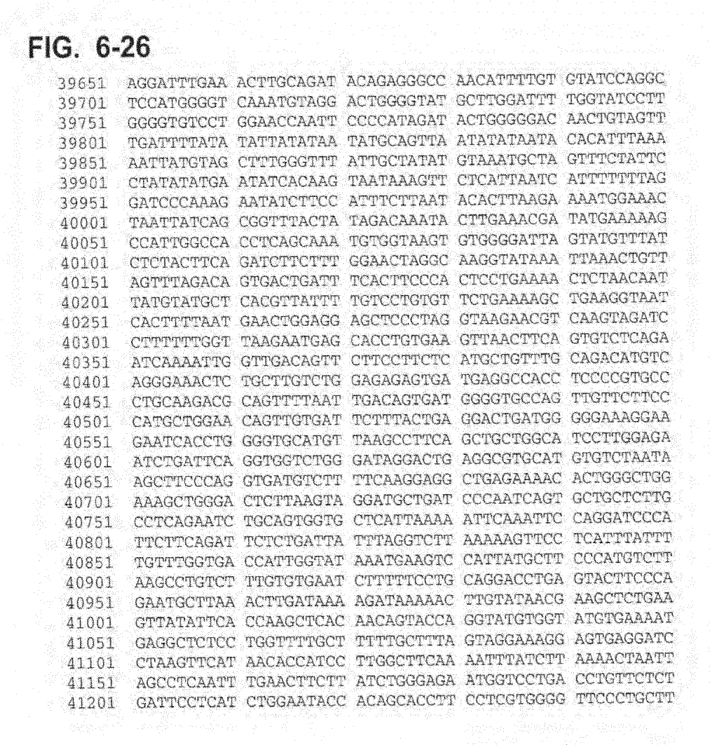

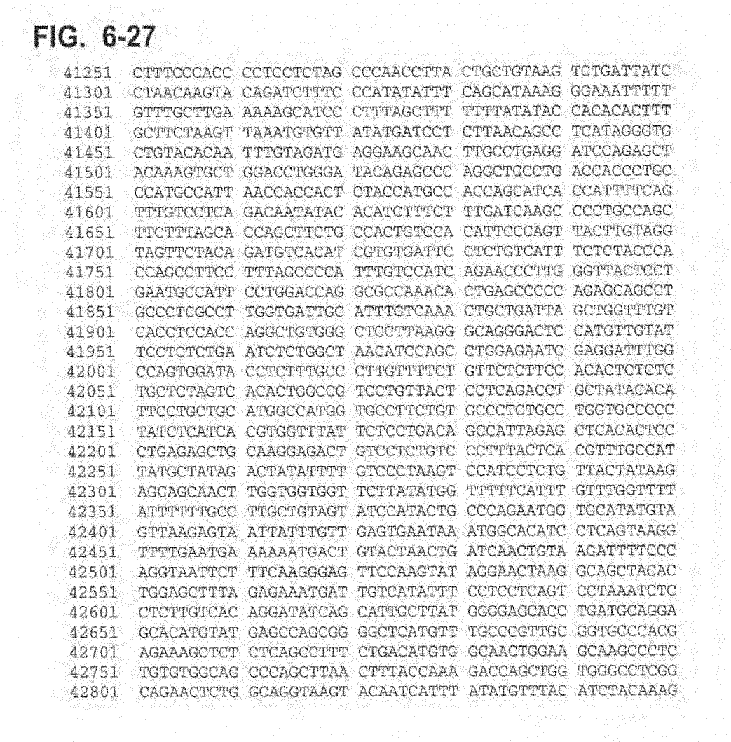

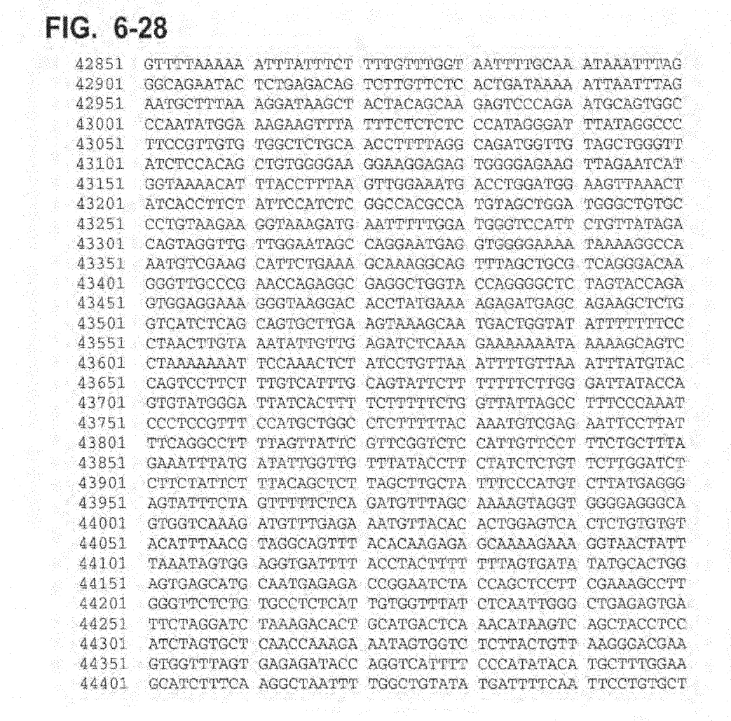

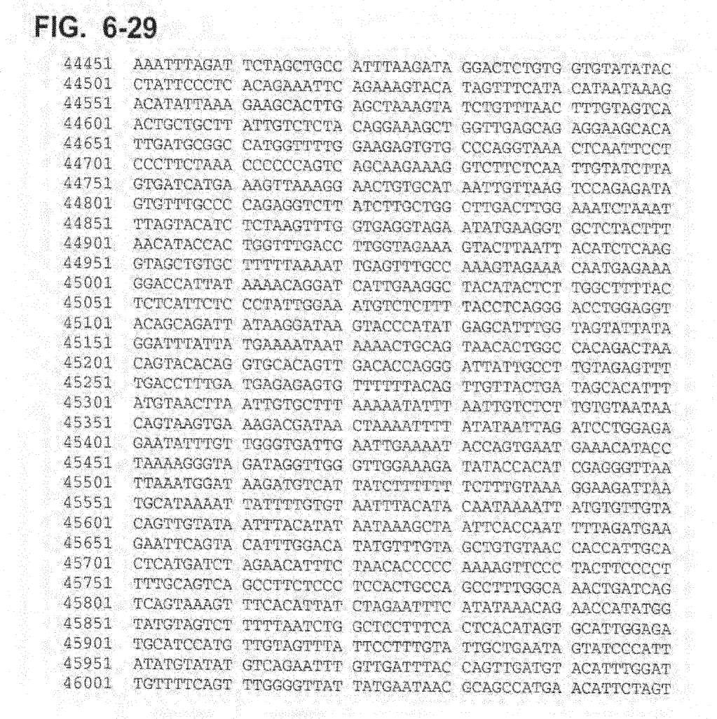

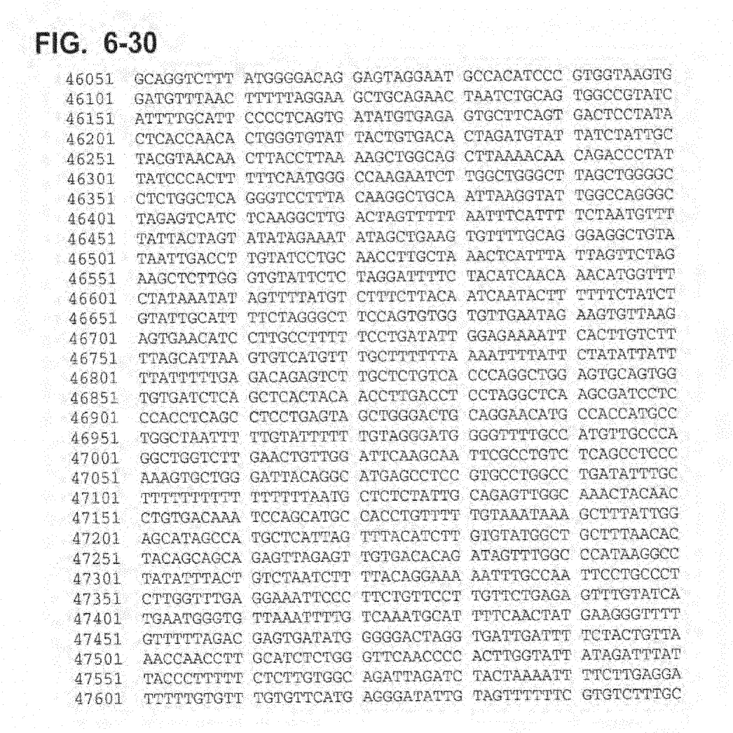

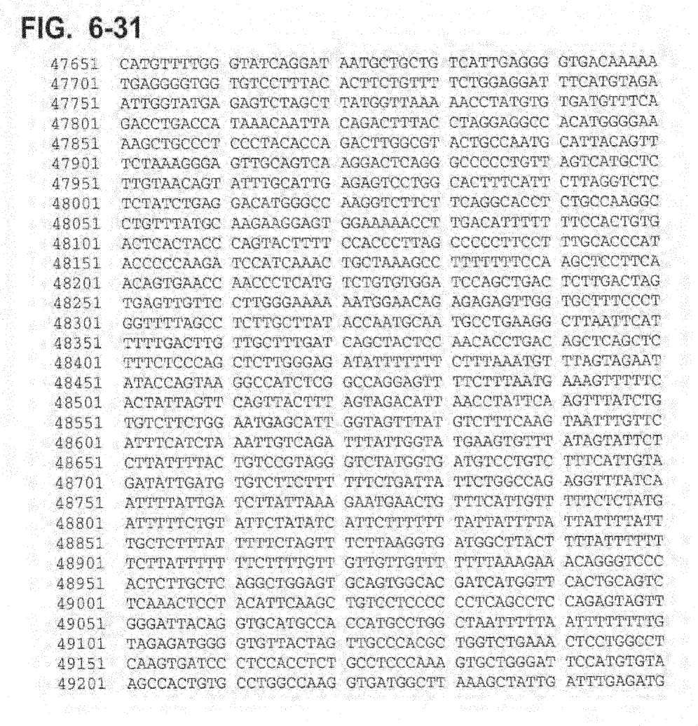

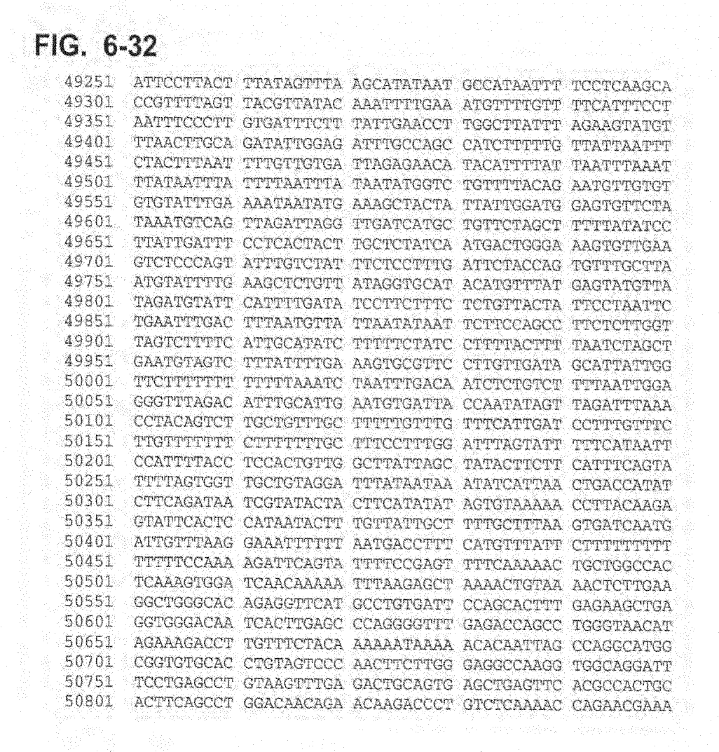

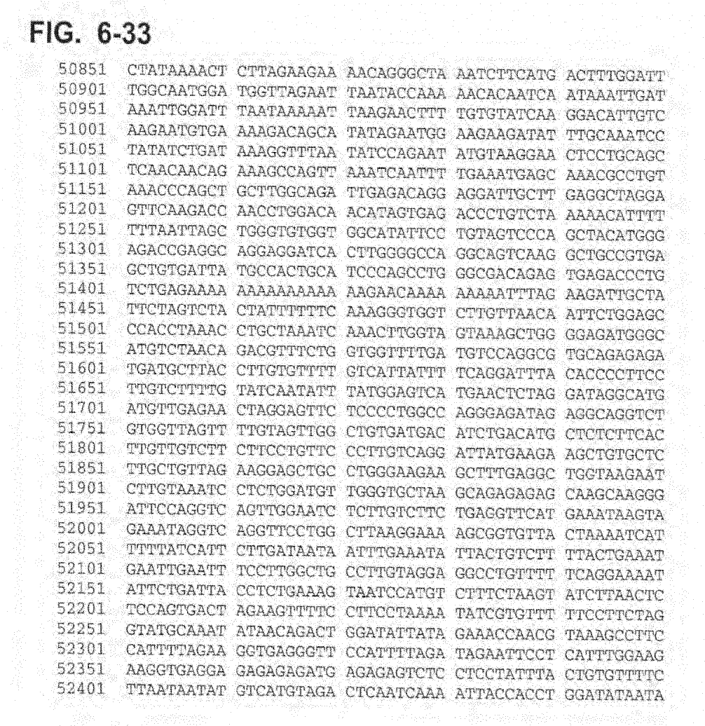

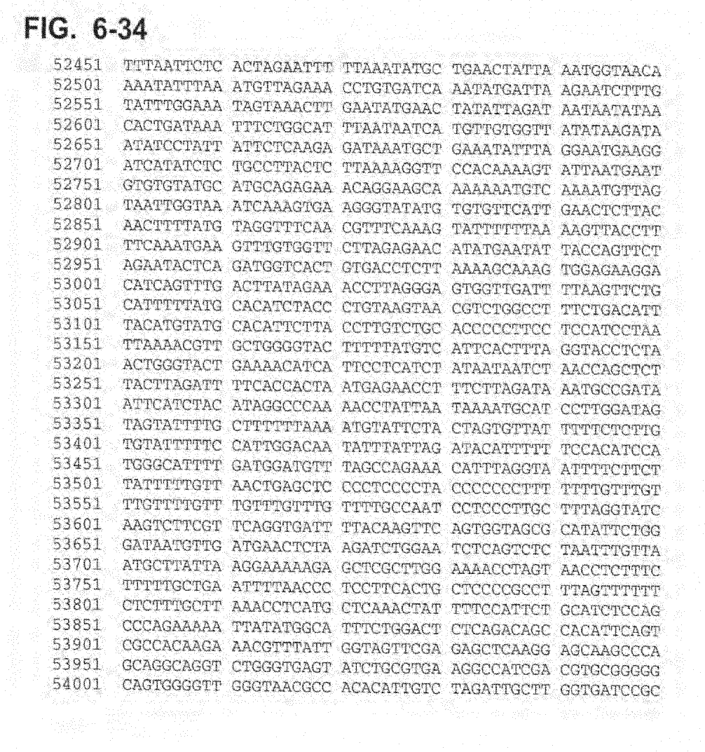

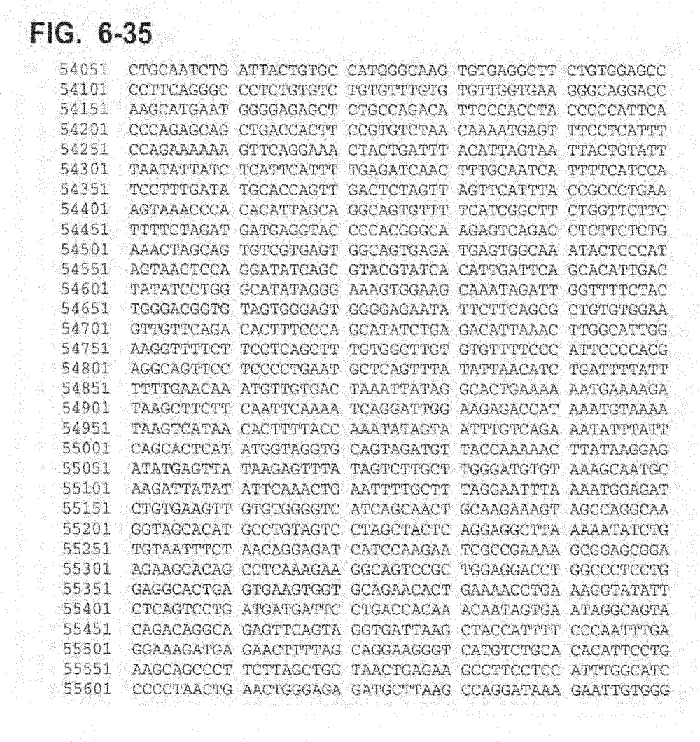

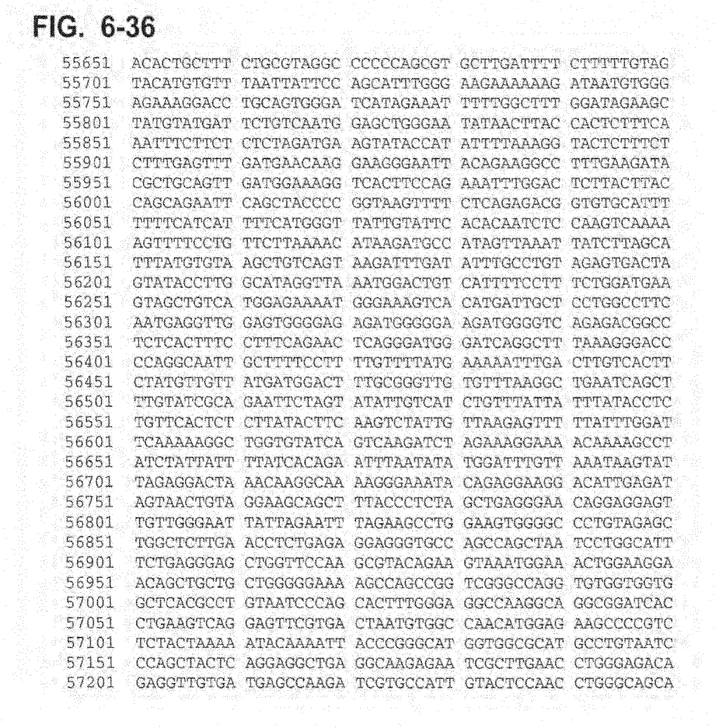

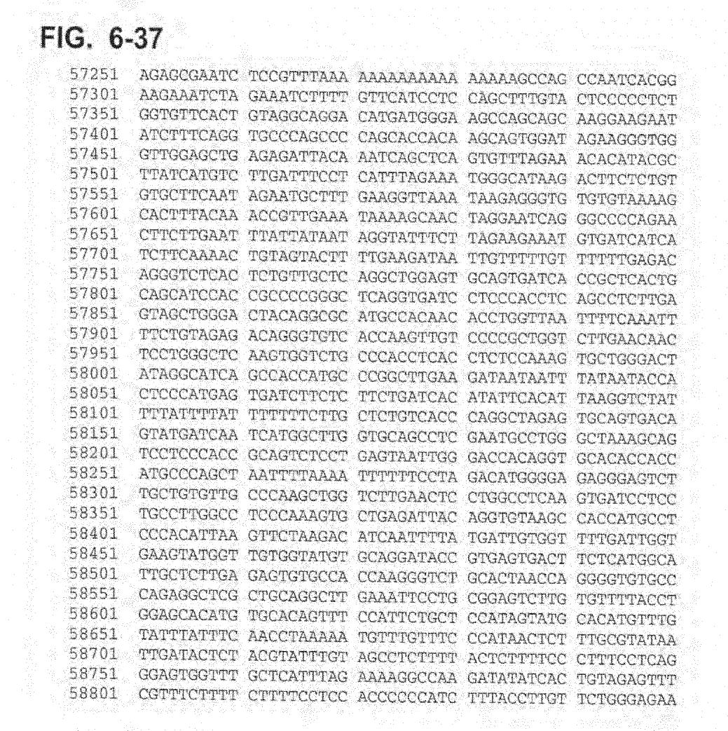

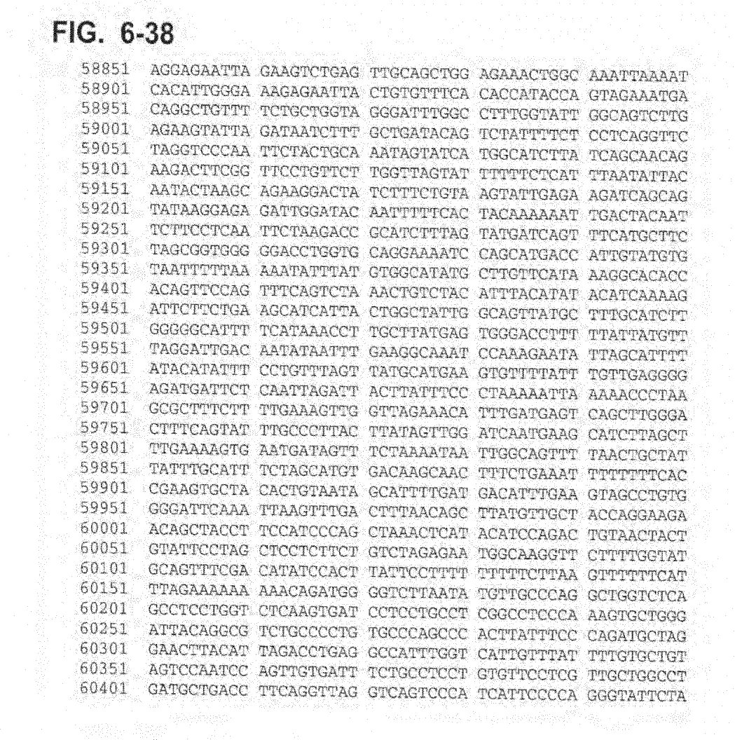

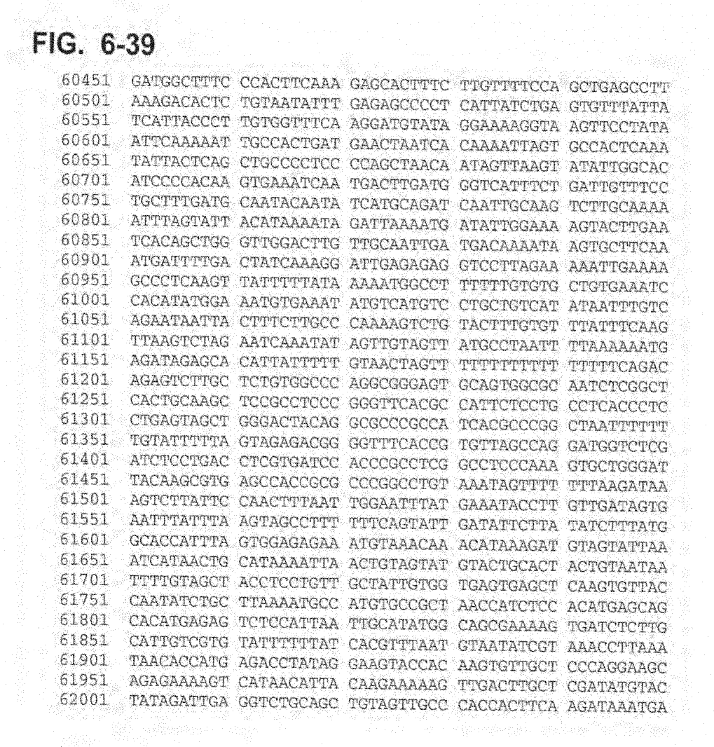

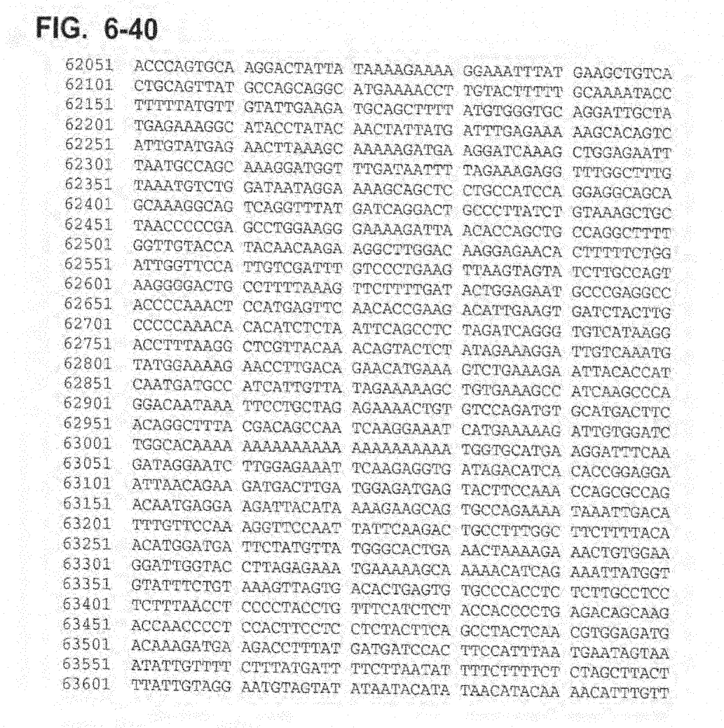

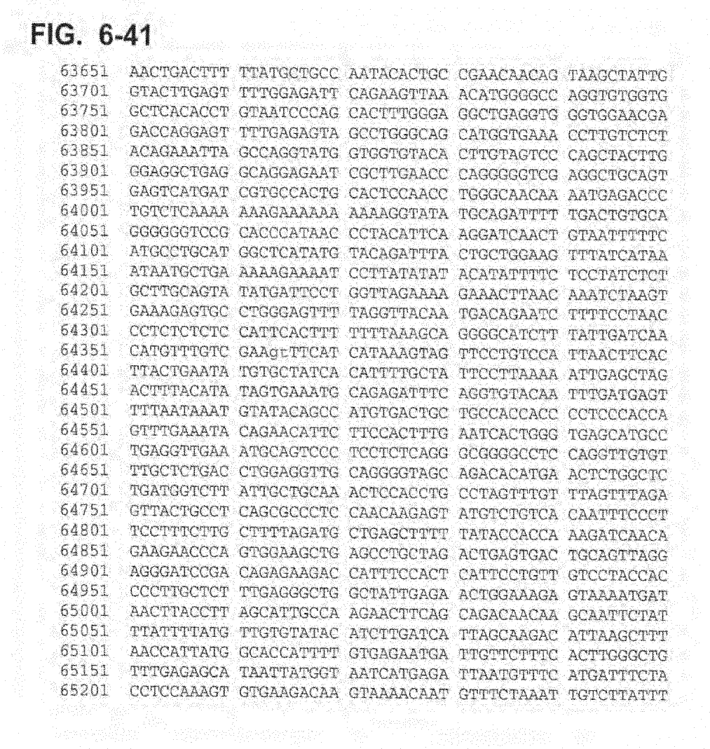

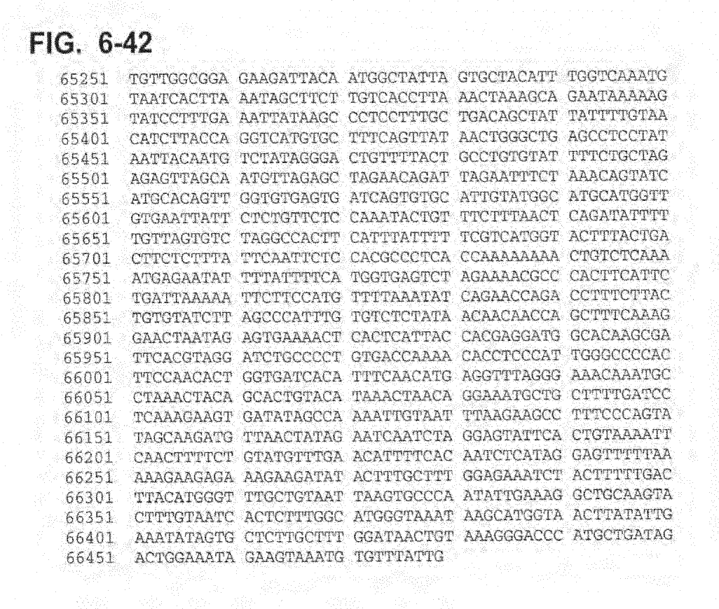

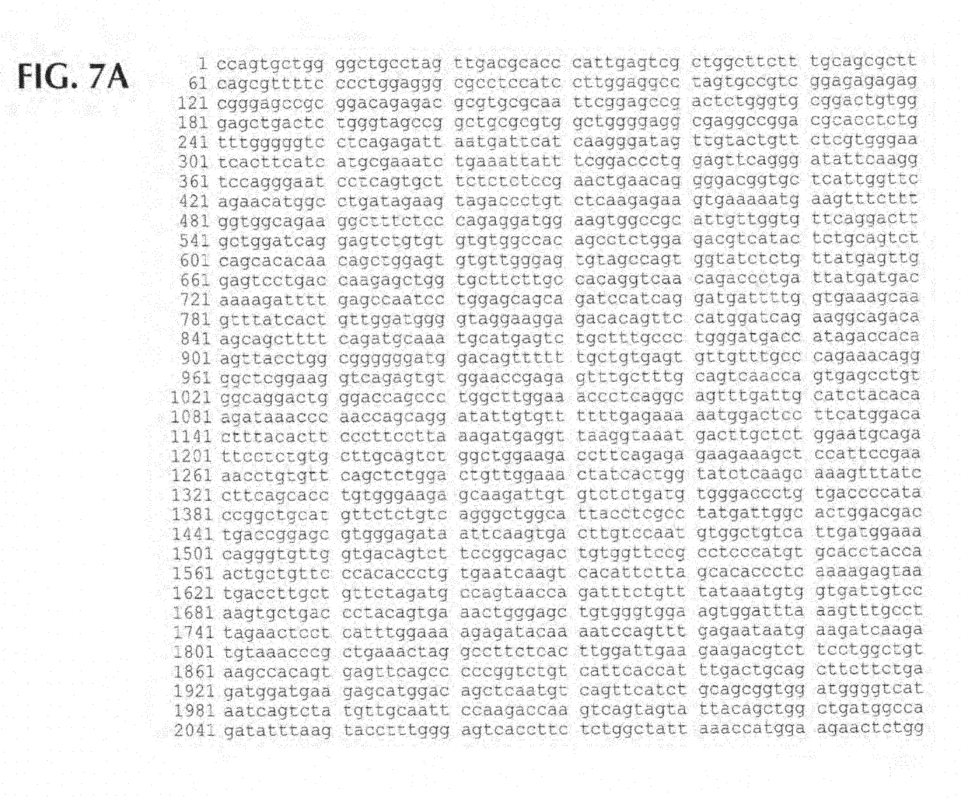

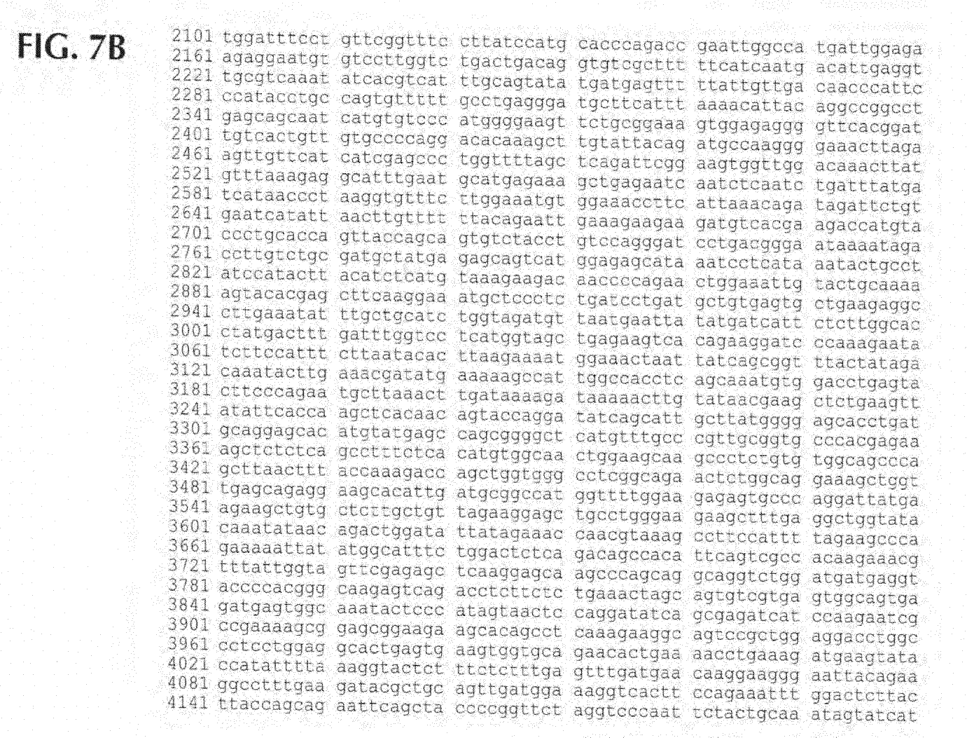

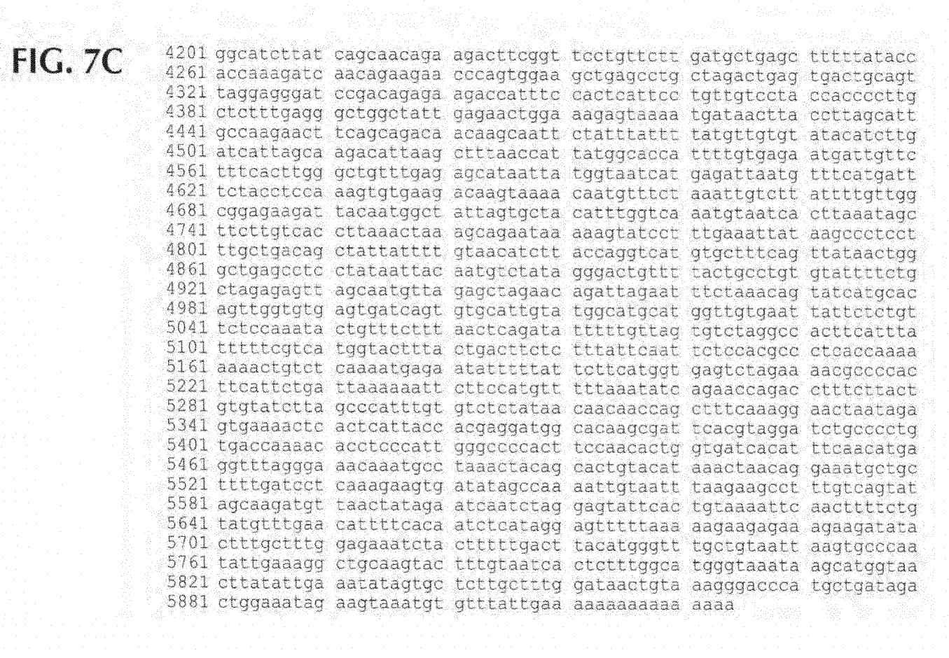

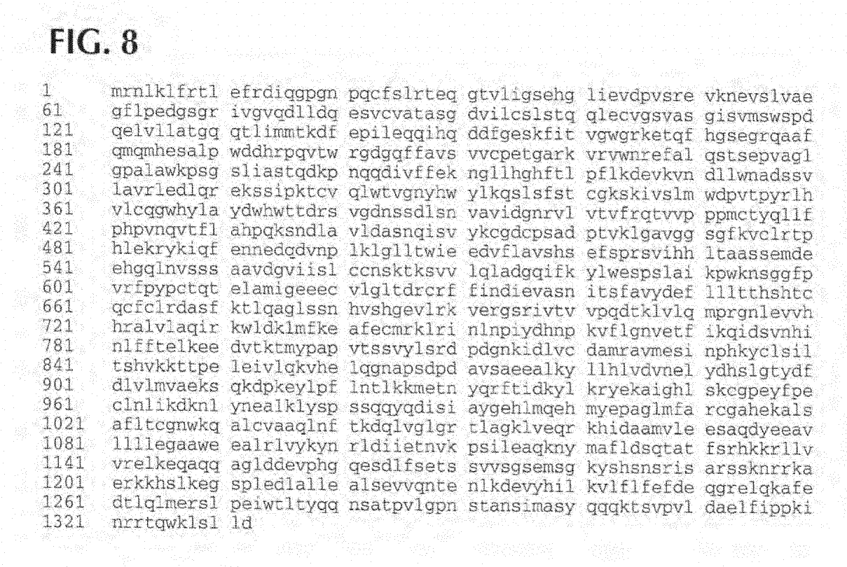

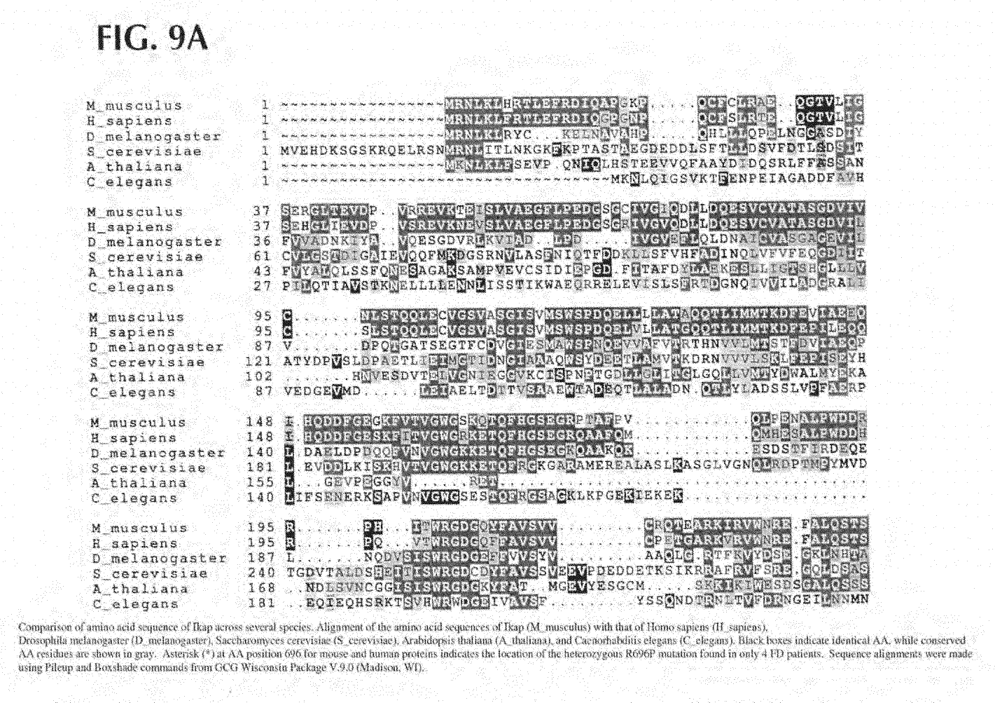

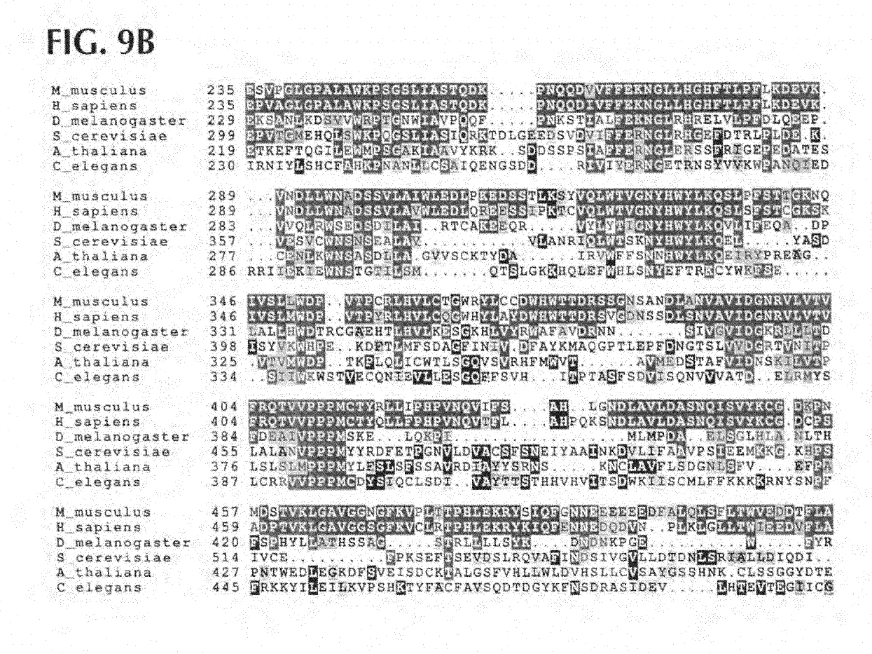

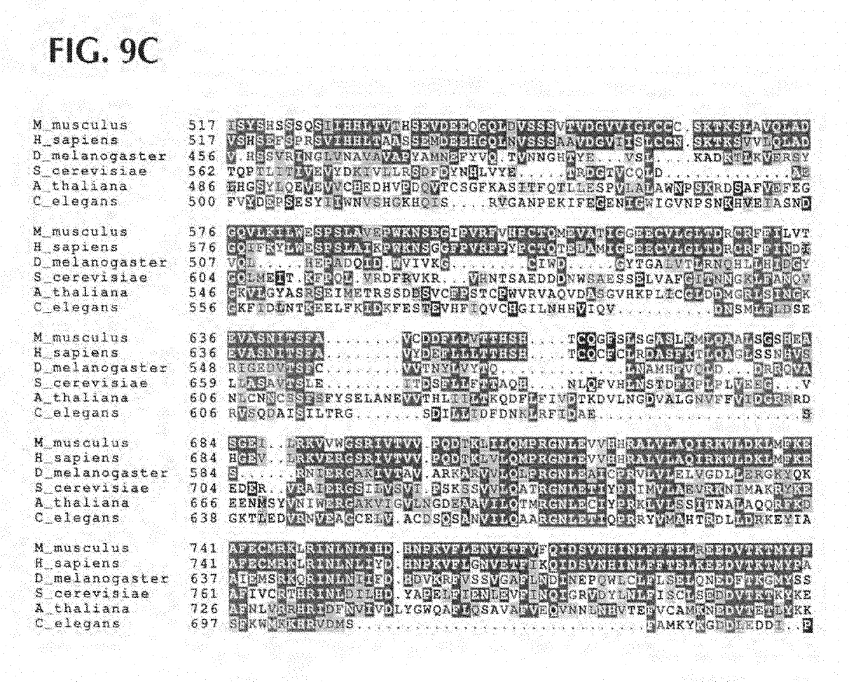

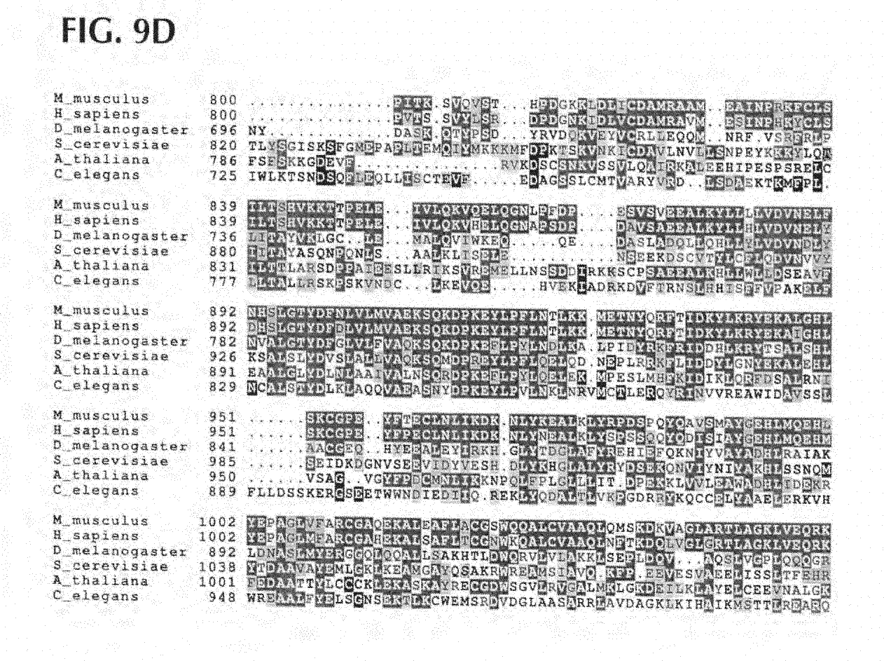

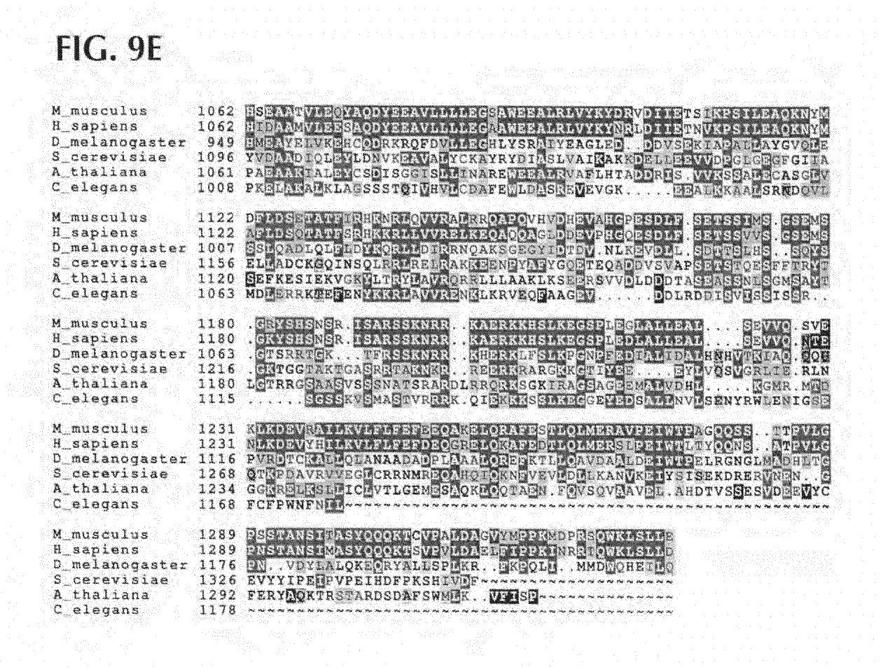

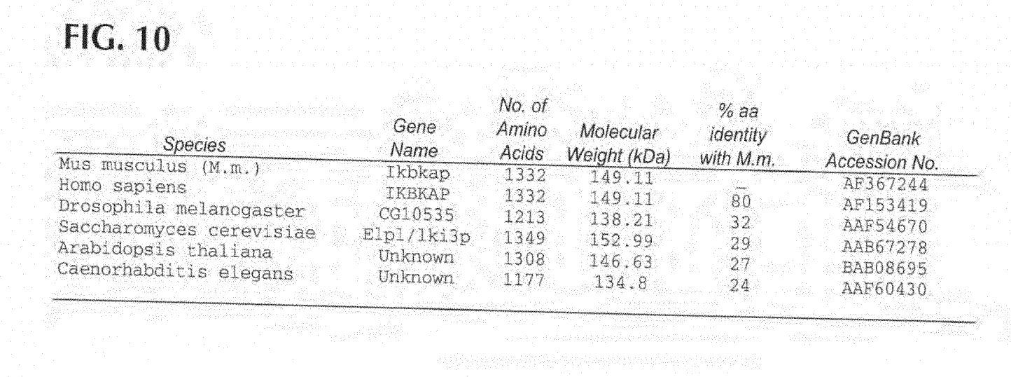

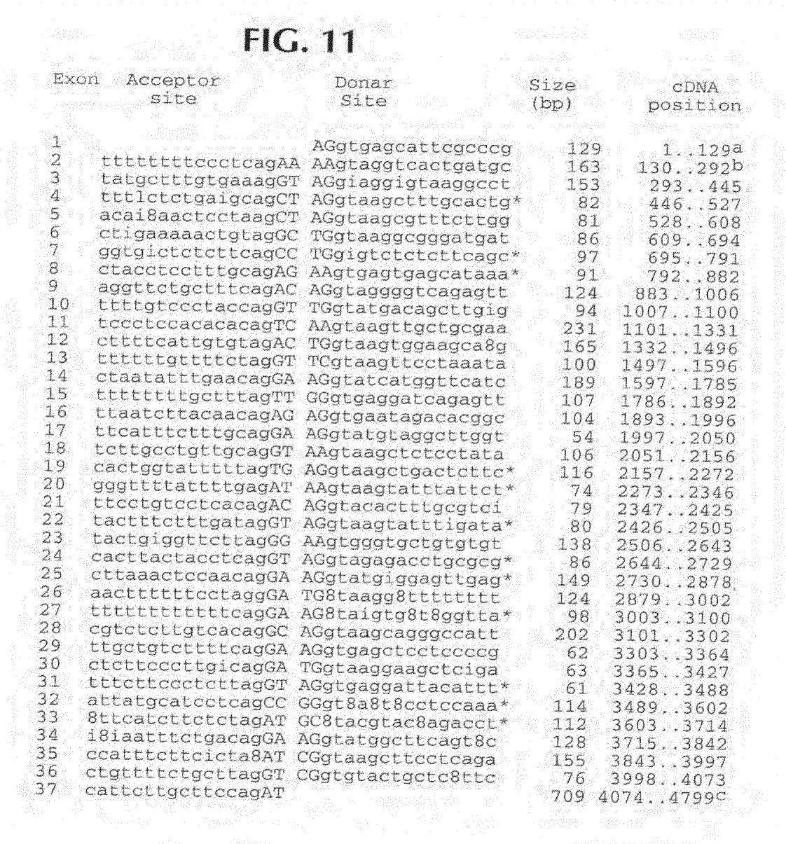

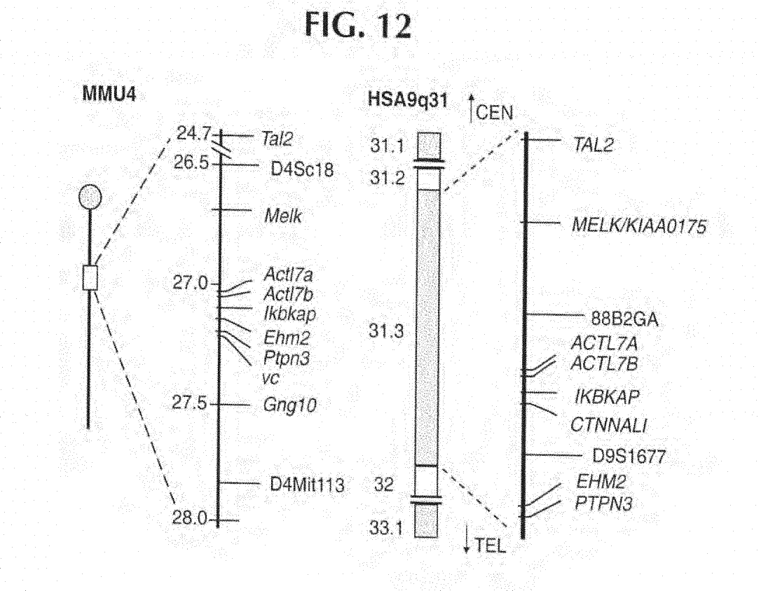

In view of the strong genetic evidence that this mutation must be pathogenic, it was postulated that its effect might be tissue-specific. RNA extracted from the brain stem and temporal lobe of a post-mortem FD brain sample was therefore examined. In contrast to FD lymphoblasts, RT-PCR of the FD brain tissue RNA using primers in exons 19 and 23 (expected to produce a normal product of 393 bp) revealed a 319 bp mutant product, indicating virtually complete absence of exon 20 from the IKBKAP mRNA (FIG. 5, lanes 10-11). As additional FD autopsy material could not be obtained, intensive analyses of additional lymphoblast and fibroblast cell lines were performed to determine whether exon-skipping could be detected.

Fibroblast lines from homozygous FD patients yielded variable results. Some primary fibroblast lines displayed approximately equal expression of the mutant and wild-type mRNAs while others displayed primarily wild-type mRNA. In addition, extensive examination of additional patient lymphoblast lines indicated that the mutant message could sometimes be detected at low levels. An example of the variability seen in FD fibroblasts and the presence of the mutant message in some FD lymphoblasts is shown in FIG. 5. In fact, close re-examination of FIG. 4a shows a trace of the mutant band in 2 (lanes 1 and 2) of the 3 FD samples. The absence of exon 20 in the FD brain RNA and the preponderance of wild-type mRNA in fibroblasts and lymphoblasts indicate that the major FD mutation acts by altering splicing of IKBKAP in a tissue-specific manner.

To identify the mutations associated with minor haplotypes 2 and 3, (Blumenfeld et al. 1999) we amplified each IKBKAP exon, including adjacent intron sequence, from genomic DNA. A single G→C change at bp 2397 (bp 73 of exon 19) that causes an arginine to proline missense mutation (R696P) was identified in all 4 patients with minor haplotype 2 (FIG. 2B). This was subsequently confirmed by RT-PCR in lymphoblast RNA as shown in FIG. 2C for a region that crosses the exon 19-20 border. The PCR product, generated from an FD patient who is a compound heterozygote with minor haplotype 2 and the major haplotype, clearly shows that RNA is being expressed equally from both alleles based on heterozygosity of the G→C point mutation in exon 19. However, the RNA from the major haplotype allele shows no evidence for skipping of exon 20 which would be expected to produce a mixture of exon 20 and 21 sequence beginning at the end of exon 19. This confirms our previous observation that lymphoblasts with the major FD mutation produce a predominance of normal IKBKAP transcript.

The R696P mutation is absent from 500 non-FD chromosomes, and it has been seen only once in 706 random AJ chromosomes in an individual who also carries the minor haplotype.

This mutation is predicted to disrupt a potential threonine phosphorylation site at residue 699 identified by Netphos 2.0 (Blom et al. 1999), suggesting that it may affect regulation of IKAP. Interestingly, the presence of this minor mutation is associated with a relatively mild disease phenotype, suggesting that a partially functional IKAP protein may be expressed from this allele. No mutation has been identified for minor haplotype 3, which represents the only non-AJ putative FD chromosome.

### Example 2—FD Diagnostic Assays

As discussed above, the allele-specific oligonucleotide (ASO) hybridization assay is highly effective for detecting single nucleotide changes in DNA and RNA, such as the T→C or G→C mutations or sequence variations, especially when used in conjunction with allele-specific PCR amplification. Thus, in accordance with the present invention, there is provided an assay kit to detect mutations in the FD gene through use of a PCR/ASO hybridization assay.

### PCR Amplycation

Genomic DNA samples are placed into a reaction vessel(s) with appropriate primers, nucleotides, buffers, and salts and subjected to PCR amplification.

Suitable genomic DNA-containing samples from patients can be readily obtained and the DNA extracted therefrom using conventional techniques. For example, DNA can be isolated and prepared in accordance with the method described in Dracopoli, N. et al. eds. Current Protocols in Human Genetics pp. 7.1.1-7.1.7 (J. Wiley & Sons, New York (1994)), the disclosure of which is hereby incorporated by reference in its entirety. Most typically, a blood sample, a buccal swab, a hair follicle preparation, or a nasal aspirate is used as a source of cells to provide the DNA.

Alternatively, RNA from an individual (i.e., freshly transcribed or messenger RNA) can be easily utilized in accordance with the present invention for the detection of the FD2 mutation. Total RNA from an individual can be isolated according to the procedure outlined in Sambrook, J. et al. Molecular Cloning—A Laboratory Manual pp. 7.3-7.76 (2nd Ed., Cold Spring Harbor Laboratory Press, New York (1989)) the disclosure of which is hereby incorporated by reference.

In a preferred embodiment, the DNA-containing sample is a blood sample from a patient being screened for FD.

In amplification, a solution containing the DNA sample (obtained either directly or through reverse transcription of RNA) is mixed with an aliquot of each of dATP, dCTP, dGTP and dTTP (i.e., Pharmacia LKB Biotechnology, N.J.), an aliquot of each of the DNA specific

PCR primers, an aliquot of Taq polymerase (i.e., Promega, Wis.), and an aliquot of PCR buffer, including MgCl₂ (i.e., Promega) to a final volume. Followed by pre-denaturation (i.e., at 95° C. for 7 minutes), PCR is carried out in a DNA thermal cycler (i.e., Perkin-Elmer Cetus, Conn.) with repetitive cycles of annealing, extension, and denaturation. As will be appreciated, such steps can be modified to optimize the PCR amplification for any particular reaction, however, exemplary conditions utilized include denaturation at 95° C. for 1 minute, annealing at 55° C. for 1 minute, and extension at 72° C. for 4 minutes, respectively, for 30 cycles. Further details of the PCR technique can be found in Erlich, “PCR Technology,” Stockton Press (1989) and U.S. Pat. No. 4,683,202, the disclosure of which is incorporated herein by reference.

In a preferred embodiment, the amplification primers used for detecting the T→C mutation and the G→C mutation in the FD gene are 5′-GCCAGTGTTTTTGCCTGAG -3′/5′-GACTGCTCTCATAGCATCGC-3′ and 5′-CGGATTGTCACTGTTGTGC-3′/5′-GACTGCTCTCATAGCATCGC-3, respectively.

### Hybridization

Following PCR amplification, the PCR products are subjected to a hybridization assay using allele-specific oligonucleotides. In a preferred embodiment, the allele-specific oligonucleotides used to detect the mutatons in the FD gene are as follows:

5′-AAGTAAG(T/C)GCCATTG-3′
and
5′-GGTTCAC(G/C)GATTGTC.

In the ASO assay, when carried out in microliter plates, for example, one well is used for the determination of the presence of the normal allele and a second well is used for the determination of the presence of the mutated allele. Thus, the results for an individual who is heterozygous for the T→C mutation (i.e. a carrier of FD) will show a signal in each of the wells, an individual who is homozygous for the T→C allele (i.e., affected with FD) will show a signal in only the C well, and an individual who does not have the FD mutation will show only one signal in the T well.

In another embodiment, a kit for detecting the FD mutation by ASO assay is provided. In the kit, amplification primers for DNA or RNA (or generally primers for amplifying a sequence of genomic DNA, reverse transcription products, complementary products) including the T→C mutated and normal alleles are provided. Allele-specific oligonucleotides are also preferably provided. The kit further includes separate reaction wells and reagents for detecting the presence of homozygosity or heterozygosity for the T→C mutation.

Within the same kit, or in separate kits, oligonucleotides for amplification and detection of other differences (such as the G→C mutation) can also be provided. If in the same kit as that used for detection of the T→C mutation, separate wells and reagents are provided, and homozygosity and heterozygosity can similarly be determined.

In another embodiment a kit combining other diseases (i.e., Canavan's)

### Example 3—FD Diagnostic: Other Nucleotide Based Assays

As will be appreciated, a variety of other nucleotide based detection techniques are available for the detection of mutations in samples of RNA or DNA from patients. See, for example, the section, above, entitled “Nucleic Acid Based Screening.” Any one or any combination of such techniques can be used in accordance with the invention for the design of a diagnostic device and method for the screening of samples of DNA or RNA for FD gene mutations in accordance with the invention, such as the mutations and sequence variants identified herein. Further, other techniques, currently available, or developed in the future, which allow for the specific detection of mutations and sequence variants in the FD gene are contemplated in accordance with the invention.

Through use of any such techniques, it will be appreciated that devices and methods can be readily developed by those of ordinary skill in art to rapidly and accurately screen for mutations and sequence variants in the FD gene in accordance with the invention.

Thus, in accordance with the invention, there is provided a nucleic acid based test for FD gene mutations and sequence variants which comprises providing a sample of a patient's DNA or RNA and assessing the DNA or RNA for the presence of one or more FD gene mutations or sequence variants. Samples of patient DNA or RNA (or genomic, transcribed, reverse transcribed, and/or complementary sequences to the FD gene) can be readily obtained as described in Example 2. Through the identification and characterization of the FD gene as taught and disclosed in the present invention, one of ordinary skill in the art can readily identify the genomic, transcribed, reverse transcribed, and/or complementary sequences to the FD gene sequence in a sample and readily detect differences therein. Such differences in accordance with the present invention can be the T→C or G→C mutations or sequence variations identified and characterized in accordance herewith. Alternatively, other differences might similarly be detectable.

Kits for conducting and/or substantially automating the process of identification and detection of selected changes, as well as reagents utilized in connection therewith, are therefore envisioned in accordance with the invention of the present invention.

As discussed above, through knowledge of the gene-associated mutations responsible for FD disease, it is now possible to prepare transgenic animals as models of the FD disease. Such animals are useful in both understanding the mechanisms of FD disease as well as use in drug discovery efforts. The animals can be used in combination with cell-based or cell-free assays for drug screening programs.

### Example 4—Creating Animal Models of FD

The first step in creating an animal model of FD is the identification and cloning of homologs of the IKBKAP gene in other species.

Isolation of Mouse cDNA Clones
The human IKBKAP sequence (GenBank Accession No. AF153419) was used to search the mouse expressed sequence tag database (dbEST) using the BLAST program (www.ncbi.nlm.nih.gov/BLAST). A single 5′ EST from a mouse brain library (GenBank Association No. AU079160) was identified that showed marked similarity to the 5′ end of

IKBKAP. The corresponding cDNA clone, MNCB-3931, was obtained from the Japanese Collection of the Research Bioresource/National Institute of Infectious Disease. In addition, eight EST's that were similar to the 3′ end of the ORF were found to belong to UniGene cluster Mn.46573 (www.ncbi.nlm.nih.gov/Unigene). Examination of this cluster yielded several poly (A+)-containing clones, and we obtained the clone UI-M-CG0p-bhb-g-07-0-U1 (GenBank

Accession No. BE994893) from Research Genetics.

### RT-PCR Analysis

RNA (1 μg/ml from BALB/c mouse brain was obtained commercially (Clontech). Oligo-dT 15 and random hexamer primers were annealed to the template at 65° C. for 10 min in the presence of 1× first-strand buffer, 2 mM dNTP mix, and 4 mM DTT. The reaction mixture was incubated at 42° C. for 90 min after addition of Superscript TM II RT (200 U/ul) and Rnase inhibitor (80 U/ul) (GIBCO).

### DNA Sequencing and Analysis

DNA sequencing was performed using the AmpliCycle sequencing kit (Applied Biosystems) for the 33[P]-labeled dideoxynucleotide chain termination reaction, using the following conditions: 30 sec at 94° C., 30 sec at 60° C., and 30 sec at 72° C. for 30 cycles. The radioactively labeled sequence reaction product was denatured at 95 C for 10 min and run on a denaturing 6% polyacrylamide gel for autoradiography. Basic sequencing manipulations and alignments were carried out using a program from Genetics Computer Group (GCC; Madison, Wis.). The cDNA sequence generated throughout the experiments were aligned and assembled into a 4799-bp cDNA named Ikbkap.
   Isolation of Full-Length cDNA

To obtain the full-length cDNA sequence, PCR was performed on the mouse cDNA template using primers designed from the sequence of the 5′- and 3′-cDNA clones. The PCR conditions were as follows: 15 sec at 95° C., 30 sec at 54° C. to 60° C., and 3 min at 68° C. for 9 cycles; then 15 sec at 95° C., 30 sec at 54 to 60° C., and 3 min with increment of 5 sec for each succeeding cycle at 68 C for 19 cycles, followed by 7 min at 72° C. The PCR products were electrophoresed on a 1% agarose gel stained with ethidium bromide and were cleaner using a Qiaquick PCR cleaning kit (Qiagen) in the preparation for cycle sequencing. Successive primers were designed in order to obtain the full-length Ikbkap sequence, which was deposited in GenBank under Accession No. AF367244.

### Northern Blot Analysis

Expression of Ikbkap was examined using both mouse embryo and adult mouse multiple tissue Northern blots (Clontech). The blots were probed with a 1045-bp PCR fragment that contains exons 2 through 11, which was generated using primer 1 (5′-GGCGTCGTAGAAATTGC-3′) and primer 2 (5′-GTGGTGCTGAAGGGGCAGGC-3′). The probe was radiolabeled (Sambrook et al., 1989) and was hybridized according to the manufacturer's instructions.

### Chromosome Mapping of the Mouse Ikbkap Gene

Several of the mouse Ikbkap ESTs belonged to the Unigene cluster Mn.46573, which has been mapped to chromosome 4 (UniSTS entry: 253051) between D4Mit287 and D4Mit197. To assess synteny between mouse chromosome 4 and human chromosome 9, we used several resources available at NCBI (www.nbci.nlm.nih.gov/Homology).

### Determination of Genomic Structure of the Mouse Ikbkap

The 37 human IKBKAP exons were searched against the Celera database to obtain homologous mouse sequences. Approximately 130 mouse genomic fragments (500-700 bp) were obtained using the Celera Discovery System and Celera's associated database, and these fragments were assembled into seven contigs. In order to assemble the complete genomic sequence, we obtained six mouse bacterial artificial chromosomes (BACs) from Researcg Genetics after they screened an RPCI-23 mouse library using 4300bp human probe that contained exon 2. To verify that these BAC clones contained the entire Ikbkap gene, we amplified fragments from the 5′ and 3′ ends of the gene, as well as a fragment from the 3′ flanking gene Actl7b (Slaugenhaupt et al., 2001) We designed primers at the ends of each of the seven contigs constructed from the Celera data and generated PCR products from the BACs. Subsequently, we sequenced and closed five of the gaps, with the resulting two contigs assembled and deposited to Celera (Accession No CSN009).

### Creating a Targeting Vector

After cloning and sequencing the mouse homolog of the human IKBKAP gene, a targeting vector can then be constructed from the mouse genomic DNA. The targeting vector would consist of two approximately 3 kb genomic fragments from the mouse FD gene as 5′ and 3′ homologous arms. These arms would be chosen to flank a region critical to the function of the FD gene product (for example, exon 20).

In place of exon 20, negative and positive selectable markers can be placed, for example, to abolish the activity of the FD gene. As a positive selectable marker a neo gene under control of phosphoglycerate kinase (pgk-1) promoter may be used and as a negative selectable marker the 5′ arm of the vector can be flanked by a pgk-1 promoted herpes simplex thymidine kinase (HSV-TK) gene can be used.

The vector is then transfected into Rl ES cells and the transfectants are subjected to positive and negative selection (i.e., G418 and gancyclovir, respectively, where neo and HSV-TK are used). PCR is then used to screen for surviving colonies for the desired homologous recombination events. These are confirmed by Southern blot analysis.

Subsequently, several mutant clones are picked and injected into C57BL/6 blastocytes to produce high-percentage chimeric animals. The animals are then mated to C57BL/6 females. Heterozygous offspring are then mated to produce homozygous mutants. Such mutant offspring can then be tested for the FD gene mutation by Southern blot analysis. In addition, these animals are tested by RT-PCR to assess whether the targeted homologous recombination results in the ablation of the FD gene mRNA. These results are confirmed by Northern blot analysis and RNase protection assays.

Once established, the FD gene-/-mice can be studied for the development of FD-like disease and can also be utilized to examine which cells and tissue-types are involved in the FD disease process. The animals can also be used to introduce the mutant or normal FD gene or for the introduction of the homologous gene to that species (i.e., mouse) and containing the T→C or G→C mutations, or other disease causing mutations. Methods for the above-described transgenic procedures are well known to those versed in the art and are described in detail by Murphy and Carter supra (1993).

The techniques described above, can also be used to introduce the T→C or G→C mutations, or other homologous mutations in the animal, into the homologous animal gene. As will be appreciated, similar techniques to those described above, can be utilized for the creation of many transgenic animal lines

To the extent that any reference (including books, articles, papers, patents, and patent applications) cited herein is not already incorporated by reference, they are hereby expressly incorporated by reference in their entirety.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

We claim:

1. A kit for the detection of the FD2 mutation associated with Familial Dysautonomia in a sample from a human subject, said kit comprising an isolated oligonucleotide probe from the group consisting of (a) through (d) below:
(a) an isolated oligonucletotide probe consisting of at least 16 contiguous nucleotides of the portion of SEQ ID NO:1 from nucleotide 32,642 to nucleotide 36,846 which includes position 33,714 of SEQ ID NO: 1 and being suitable for the detection of the FD2 mutation at position 33,714 of SEQ ID NO:1;
(b) the complement of an isolated oligonucletotide probe consisting of at least 16 contiguous nucleotides of the portion of SEQ ID NO:1 from nucleotide 32,642 to nucleotide 36,846 which includes position 33,714 of SEQ ID NO: 1 and being suitable for the detection of the FD2 mutation at position 33,714 of SEQ ID NO: 1;
(c) an isolated oligonucletotide probe consisting of at least 16 contiguous nucleotides of the portion of SEQ ID NO:1 from nucleotide 32,642 to nucleotide 36,846 which includes position 33,714 of SEQ ID NO: 1 except that the nucleotide which is at the same position as position 33,714 of SEQ ID NO:1 is a cytosine and being suitable for the detection of the FD2 mutation at position 33,714 of SEQ ID NO:1; and
(d) the complement of an isolated oligonucletotide probe consisting of at least 16 contiguous nucleotides of the portion of SEQ ID NO:1 from nucleotide 32,642 to nucleotide 36,846 which includes position 33,714 of SEQ ID NO: 1 except that the nucleotide which is at the same position as position 33,714 of SEQ ID NO:1 is a cytosine and being suitable for the detection of the FD2 mutation at position 33,714 of SEQ ID NO: 1.

2. The kit of claim 1, wherein the isolated oligonucleotide probe is 16 nucleotides.


SEQUENCE LISTING (89 sequences)

SEQ ID NO: 1 - DNA, 66479 bp, Homo sapiens
ccagtgctgcggctgcctagttgacgcacccattgagtcgctggcttctttgcagcgcttcagcgttttcccctggagggcgcctccatccttggaggcctagtgccgtcggagagagagcgggagccgcggacagagacgcgtgcgcaattcggagccgactctgggtgcggactgtgggagctgactctgggtagccggctgcgcgtggctggggaggcgaggccggacgcacctctgtttgggggtcctcaggtaagcgatccatccagggtaggggcacgggagtggacctctccgccggcggtgtccgggtgaaggagacccggagcctcctctgcctgctgcgggccggggactggagtgcgggctgcaccacctctttcctagagccttaaattctttttgcagccttgccacctgctccatcgggggcgctgggaggcgcgacagcccagggatgcctgctgcccctccagccggacttaacccagcctcttgattgcttgcagggggttgataataacgctgaaagcgagagtattaattcacgatggaaggcggcggttaatagaggctcgggtgctgtggtgcgggtcctttctcgcgtgtgagactttttcgtggaggtggtgtcctctgtgcttctccatctaacgtggtgttttacgtggctttctctcccgttaacgatgatctccgtggagacagtggctgagtaatcttcagatcccagtacttagcaagtgctcagtcggtgttggatgtaggccacaaaccggatcgtaaagaattcaactgtatattgacagccacggaactaatcaatgaatagatccgtatgaagagtaagcaaaaaggcagcaaagacagtttttcagcttggggacatagagtagaaatggtctgtccccaaatagtgggaactgtcatttgggggaagaatagcaagttctttgctttccaggtcgcatttgatgtgcatgtgagacatgcttgtgattctatcaggaggttgaaaatgtgggtttagtggtaagtttgggctaattcagtcagggctaggcatttaggcctaatcagcgtattggtgatctacctggtatatgtaatcatgcatgtgatgtctagccaagaggtggatagtcgaaggagcaagggaagaaaatgaagcagttatcaggaaattaagagagaatccacgattgacctttggtgtggagggatctttagcacatttaagaactgcgaagagtttgaatcagtggaggcaggaaggttggaggttgcagatgtccaagaaagagtactaataggcctaggtcctgtggcaatatggaggatattcctttcctagcctggaaagaagtggagggaagtcttcctccgagaagataagggaataaggctgatgggtgtgaaatttcagagaaactagttttgaggcgtttttatgatgtttaaagatgaaaaacgagcaggcacggtggctcaggcctgtaatcccagcactttgggaggcagaggcgggtggatcacttgaggttaggagttcaagaacagcctgggcaacatggtgaaaccctgtctctactaaaaatacaaaaattaactgggcatggtgccgggcgcctgtaatcccagctactccggaggctgaggcaggagaatcgcttgaacccggaaggcagatgttgcggtgagccgagatcgcgccattgcaccccagcctgggcaataagagcgaaactccgtctcaaaaaacaaaaaaacctgcatgatatgttagaggttcaagtaatttctagcagttcttgaatataattgtcaccaaaacttactaaaatcattgtcttcctcacttccatcatatataaacttacctttctcttatcccacattatatattatataattcctatgacacttgacattatcttctgtgtactattaggattgattcatctttattctttctatgtcatacatatgtggggtgccaagatgagagaagtctccttggattaaagtgacaataagaccggtgtggtccttgtaattgctacccctaacataagttagggacttacaatcataagccttaaagggatctgaatataaataactagcacagtaacatttttttcccctacttaggtaatgttatgcatttaagcaagcctgattttgccagaccaaagtagatgtcttgtttagcactcttttctcacgttttatattgtcctgggaaaagcctggccagaagaacaaagttactggaagtagttatgtcaggtcatcagggtccttgaaatgttggtcatcattttgaagtaaattgttgtcatgtcccagtattttctcttcccctttagaacagtaaatgcttttctatctttgatttcagtttttttatgaatgtataaaaccagtttataaatgaatagacctggtgaatattaaagtcatttcagattctcttcaactgccagtatataaaaatggattttcaaatagtgctaatcagtgggatacccttttgtttttcctcatgattttataaagatgtcctaatatgcaaaaataaaatgtttccccattcatttgttctttcaactttcccaaaggaataactgatattacatcttttttgaagaaaacattctaaagttgagaatcttgcctctcctaaaaagaacataaaataggtttcagaattcctaatttgtagaccataactgtatagagtgggtcaggttgctgctataatccatacatgggtgtgtactcagagaggtaagttttttcttttcttggttattctgattctgactaccacttcttcaccccctgaatcatttcatttaaataaatatggtcatttatcactattaagctatttatttttctcttagagattaatgattcatcaagggatagttgtacttgtctcgtgggaatcacttcatcatgcgaaatctgaaattatttcggaccctggagttcagggatattcaaggtccagggaatcctcagtgcttctctctccgaactgaacaggggacggtgctcattggttcagaacatggcctgatagaagtagaccctgtctcaagagaagtaagttactgatgtagaatgccagcatgtgggtatgacccttgatttctcttcttccaaatttctttccccacatggtctttctttatatcttattgaatttatatcctcccaaataaacatcttttgcttcatatatatgccatgttagacatagcttaaatcgtaatccttctttaactctgctgctattttaacctaagtcagtagaactctgaccttactttttgagtgtgtgccgtactttttaccctctttgtcatgcaaattctgtttataagagtggtttttttttttttttttttgagacggagtctcgctctgtcacccaggctggagtgcagtggtgtgatcgtggctcactgcaagctccgcctccccgggttcacaccattctcctgcctcagcctcccgagaagctgggactacaggcgcccgccaccgcgcccggctaattttttgtatttttagtagatgtgcggtttcaccgtgttagccaggatggtcttgatctcctgacctcgtgatccgcctgcctcagcgcccggccaagagtggtttttaattgggaatgaacacgaaagttgcccatggagctttctaaaagtttgagcccacatctcatgtcaactaaatcagaatctttagtgttggctcctaactatatgtactttaaaaacctctgtgggttggttttgatatggtcccttgattatgttcttctactaatacattttaggcagttacatcctttagtgccttttccccatactatagaaatcttagaaaagcatagctattagcatcatattttagtggacaattttaaagagaccaggcttattgtttttgtttttgtgtttgtttggcaaaaaggtcacattacctatttttcttgttagagatgacagagtagtgatatttctcaaatgaaagtttggattttcatctagaaaaaatatttttgaaagcttttatgtaataaaagaagcattaaaaagtatttctggaaatgttatcaattattcttgaaagtagactgggttaatttgcttgtgtttactttggtgaaaggtgaaaaatgaagtttctttggtggcagaaggctttctcccagaggatggaagtggccgcattgttggtgttcaggacttgctggatcaggagtctgtgtgtgtggccacagcctctggagacgtcatactctgcagtctcagcacacaacaggtaagtggaagactccagtgaggggggagtctcaagcatcctcaaataggttacttgctatttgtggaagttttcaaatcagtagccataatagttacacttttgctaattaatttttgcattatatatttctttatttaaaaaattgttaacatggctttatctatatgttaagattcttctaaaactgagttttgtctgctgcatctattaatcagagtgatcagaatgttccaaatgagaatatatttttttaaaagttaaaactggctattcttatgtggtgtagatcacctcttatcagaccctcatcttgagttgcaacctttgtttctcaatttaggaagtctttgtttatctgacttagattttctgttatgaatgttgattggctaaatttagagtccctgaagtctaggcactaaagtaaatacattgtcattacctgcacatgtgatgactgccagtagagctagacttcaagcaattgcttctttctctactttagtgtatagttgagtttctgatttctatcctcaccttcttaacagcaagggtttcaaattacacttggctgattctttaaatcttcttccattacttcattagttgtgatctccttaacattgattatgtcacagaagttagagtattactaatagtaggataatgatagcagcttacatttattaactatcatgtgcctggcactttttaaagtgcttttcatgcaaatttatttaatcttcaccatgaccttatgcagtaggttgttgtttcctattcttcagaagaggcagttaaggcacagagtgcttaagtaattagaccagggtcacacagtaatcaaatggggtttgaccctagcagtctaaatctggcacctctgctcttaaccattccatttagtacaatcataaacctttacttgcagttcatggtgggaaatatcaaacttgtcatatacagcttgtttttttttcgtatttgaaagatagatgcttttactttccaaacattttgtagcattgtttcctggttactgagctcttccagtctatttatcttcatttaatggtgctgattctgccctttagtggcttctcaattgtctgaaaggtagagcccactattgtgccttataagcccctttcactatctgttccccacattcctttttagcctcatccccccattgttcctgtgtgtacgtaaaccttatgttttagttgcagctgatttttaactgctcttttttctggctttgtgcctctacactgtgttttcttcctggtctctctttcctgtccttattaccactctttgaaacacgtcagaaaaactttttctggactttgggccacttgtcattccctgtgctgagacgcattttgctttccagagatcttggtcattgctgttatcctctgtagggtcttcttttatctccctcgtgagacagctctgggaagaaaaagatatttatttctaatccctgtgcctaataacaggtctattctcttgatatccattactgaagaaatgtttgttgagtaagttcttgttttaatttttaaatataaatttttaatttttatgagtacatagtaggtacatatatttatgggctacatgagatgttctgatacaggcatgcagtgcaaaataaccacatcatggagaataggatatccatcccatcaagcgtttatcctttgtgttacaaacaatccaattacagtcttttagttattttaaaatgtgcaattactgttgactgtagttaccttgttgtgctatcaaatagcaggtcttatttattctatttttttttgtacctattaaccatcccaacttccctcagcccctcactacccttcccagcctctggtaaccatccttgtactctctgtgtccgtgagttcaattgttttgatttttagatcgcacaaataagtgagaacatgtgatgtttgtctttttgtgtctggtttatttcacttaatgtaatcatctccagttccatctatgttgttgcagatgacaggatcttattctttttttatggctgaatagtactccattgtgtatagtaccacaatttctttatccagtcatccattgatggacacttaggttgcttccaaatcttagctattgtgaacagagctgcaacaaacatgagagtgcagatatctcttccatatactgatgtcttttcgttttgtttttttaattgttttgattgaagttgcagtcagtttttactgagatgctagtgtttgaatctctcttttcaattttctctgtctcagctggagtgtgttgggagtgtagccagtggtatctctgttatgagttggagtcctgaccaagagctggtgcttcttgccacaggtaagcttgttactggtgcctcactggcttttttaaaacattattccagatgtcttacaggcttcatcagctttaggctgcttgaatttcaaaaaatttctttgaaccagtataataccaattatgaaccagtataataccaattatgtatgtgtgtgtgtatatatatataaaacgtagagtgatttttttttggtgactgaagttttgcctcttagtctatcattataaaaagttgtttcatgtaactttttaagtctttgggagtaagaaacaaagtcataaaacttggggaggctgctaagtccccagttagagttaaaaatgtcagcaatatgtattttaacttattctaagagttgctgtatggacacattctaaaagcccttcttgggttctgttgctgtttttcccctttaagtctcatcattccagatgagtttagtaaaccagctccactgatgacatttatatttagaggtatcttggggacaaggagtgttgaagttagtggaggagggctttgtggacttttaagttcaactgtacacacattaatagctgagcataagcaccaggtgacttatctagggaaagctttttggggttttttgtcattgttgtttttttaagtcaaagcattttggatgaattctgtctgctctgttcagactaactccagctccttagcttacagtgccataggtacttaggaatggcaaatttgttacatgaaaacaaaatcatttttgtttgtgtttctctaaggtcaacagaccctgattatgatgacaaaagattttgagccaatcctggagcagcagatccatcaggatgattttggtgaaagtaagtatagctttgtgcaatattttgtgacctacgtttcttcccatttttgaccatttccttgtgcactaatagccatgtcattaggccaaagaactgtgaaagttaaacccccagctattaaatgtctattagcccagttccttcagcccatcccaaatcttaaaaggcctactgatgcctctccaggtctgagggtttaaggtcacttagatagttattacccaaaccctaggaaagtcttaggctgggctttcagtgaaagggactgtacaaggtagtatttctgggatacagttttagggagaagaaaagaagaaagatggaatagaaggctggtttttgttactacgattagatccaatctgcatttccatgggaacaatcagattattttcttgctaaaatctagccaaggtcatctgggcattaaggctgtgggggtattgaagggcagtgcaggagaagagagacgcttattaagcataagctttggccatcttgaagtcacaaagtagctggcctgattgaagagggatggggaagaagatgttccaacttctgttatggtctaacttcctgccttcttgctccatcaactctgagaaatcatttagacaacttctacccatttatttacaaataatgtatttgttcagaaataattttggagggctgggcacagtggctcatgcctgtaatcccagcacttttggaggatgaggcaggaggattgcttgagcccaggagtttgatactagcctaggcaacgtagggagacccagcatctacaaagaatttaaaaattagctgggcttggtggtatcagcacagtaatgacatgatgtgcaggtactggggtagcataagggaaggaaacgagtaactagagagggatgatttatttcccctaggaggccaacttgagctgagtctcagctgaattggtgttgggtaggtgagggataagggtggggagtagtcagctgaattggtattgggcaggtgagggataagggtttggagtagtcagctgaattggtattgggtaggtgagggataagggtggggaacagtccaagcaagtgaatgtgtccatttcaagtgtccatttcaagggagggttatttcatagaaacattgtgggttactcagggaactgtgagtaattcagcattgctgaagtggcagaatgtgagtgtagaatgaaataaatggaacagatttgattgagtttgtagtagggaatatggacattgagttatagttgatcagccattacaagttttgatgataagaggtttaaagagatttatttaatagaaagatggctcgtgatggcatatttttgttgtttttgtgtgtggagagggaagagatgagaggcagggtgatcaggtaggaggttgctacaggaatccagatgaaagataaggaaggtttgtgtggggctagaagcaggaatcattcaggaaaaaacttgattcacaatgaggatgggagtacattttttagaattagctgggaaacttttttagaatatatgtgcatgattccccttctgccctaggccagtttgagaaataccaatttagaaagtgaaataaataggctttgcgtatgtaaggtgaataagaaaaagttgagcaggactccagccagaacctcaggtgttgggaataaagatgccagtaacagggaagatggagaagtgctggtctgtaaggggtgggtggtgagatctgttttggatttgttgaaggaccatatgtgattgccatgtggagtatgcaaatataaggctgaagctcaggagaggccagagctatggactgagagtagtgggtatgtaggaaattctgacagttttgggaacagatggactgtctcagggagcagatgctgtacaggaagagtctagaatccagggtggaactctggggcatccagctttgaggacagtcagagagagagtaacagcacacagtatactttgggatgggaaagtgctctgggcctggtgtttcccactgactttttcacacaaatcctaatgcagtaaatcaaaggaaatgtaggccaagttaagatcttaggtctcagaaatgtgtttctcagtacaaaaaaaaaaaaatcattctatggagtgatgaatatttttcctctatcctggggtcagtagacttgttctgaaaagggctaggtcatgaatatgttcagctttgcaggctgtatgatctgtgttgcagctgctcaattctaatgttgaggtgtgaaagttatacatgatacataagcacatctatgttccagtaaacgtttgtttgtaaaagcagatgtaggctgtagttttgcaaatccctgctgtaaccccatcatttcttgtcttccattggaaaagttctctttcttcattccttggtccttaatctttctgtggaaacttgcagatagaagcctgggggtttgcaccaggatagtcactaccatttgtacgcagcagcaattgaggtactgtagcacttggatgtgagcagacaggaaatggtcatatggacccataatttataggaattgcaaacagccctgcttcatcagaatcagaatcaatggcaggaggaaagtattgggtcctggattaggtgatgttttcaggaccatctttattgtgcttcttgcaaatggatcctacctccaggaacagaagggttgtgttgtttcagcaactctgcctaatagtttatataagagaagtgttacgatctagaaagaaccccagtcagcctggaaggcagaagacctgtgttctactttttggctccaccattagggagggtctcaatctctaagtctatgtgaggagctgttttgtgacctgcagcccctctatcaccagtgagagcttgcaatcagaattttattcccagttctcatcttggggttttatgttccggacatattttgtaaactctttatgtttcattcttcttacttataaggtgagggtgagatcgctgacttgtgtcatcaaagaaacttggaatatgtaagatggcagtaaaatgctttccaaaataaggaagggcatttcaaattcttcaaagtcactgctgcatataatatgaaatgggttttgtttgtttgttttgagatgggggtctcgctgtgttacccaggctagagagtgcagtagtacaatcagggctcactgcagccttgaactcctgggttcaagtgatcctcctactttagtctcttgagtagctgggaccacaggtgtgtgccatcatgtccagcttattttgtatactttttgtagagatgggtgtctccctatgttgcccaggctggtctcgaactcctggactcaagtgatcctcctgcctcagcctcccaaagtgttgggactataggcatgagccaccatgcccagcctgaaacataggtttctcaaatattgactgctggtcaatttattgagaggcgttagaggacctgagtaattgccaatgactaacttcatgaagaatagcagtgaaactgtttttgtttcatttcatgtggcttattagttgtcttgccaattgttctgtaggcaagtttatcactgttggatggggtaggaaggagacacagttccatggatcagaaggcagacaagcagcttttcagatgcaaatggtaagtttggtttgatggataaaaagccttgactggaacaaatgtaagtttgccacccaccaggaactctttggtgtccacttagatgccagtaatgaacagttctcttctgctttagtaaaactgcctagaaccttcaggaaatgaatccctctagaaagatcctttttttccttgttattgccaagttgctttgtgatttattttcatagtagcaaataattataaccaatattcatcacccagtttaaaaaataaaacatcacagacaaaggaaaccccctgtgtatcccgtcccgatgtccctccccttcctctccagagagagctgccatccttcattcacatgcatgttctcatacttttcccatatatgtgtatattagatatttttctttttctgttggatgaaactctttgttttccttacttctggattggaaaattctgaagaccatataatgatgtcttgatgactcaaggcaggactttttaatcttctaatgtaggcggggcggcccctgaaggcagaggtgtgtggacacaagaagagtgcagactcttggggcacctggggaagtagtgtccgtgtcacattaaattcatttaaactcttatattttattttaatttatacaatatgaatattttttaaaactatgaattgaaaagtattacccttgagtaaaattaatgccccaagaagatgtgccatatttaccctctggcacactaccaagtacccccaggggcattacagatctctgttagaaaagtacagattacattatcctcataacatttagaagctatgagaccttggcagggaagtttcctaatgtttctgagcctcagtattctctgtaaagtggacaacataatgtctccttacaagggttgagatgggcaggtaatagcatatataaaacagctatcatagcatcagcacagtgtaggcactcaaatggtagttgctgcttttgttttagtagacaaataatttttgaaactttttaaagcgtagtttttatttcaaaacaactttattgtgagtaaaatatgcatagtgggtctaatttaacattctgaaagctattgacttattagaacagtaaaggattattagagggcagaaacatggagtaagtactctgagacacaaccttgcttctttgggggtgatccactacaactgcccagctttggacaagtggttttcatgttcccctgatttttaagtgattttttttttttttggcaggacttaaaaggtatccttgactaaacaggaacttgaccaagtaaatagttggtgcaatttgaatattctttcttgctataagcaacaagtaaattatggtacagctttctaagaccatatcttttcgatttaaaaatagcactttactcatacatgttatgacatgggtaaacctcataaagattatgctaagtgaaagaagccagtcataaaagatcacatataatatgatcccatttgtatgaagtgcccagaaggggcaaatccacagaggcagaaagtagagtagtggttgggtagggctgtggggtggggtggggaaggggtgactgctaatggatatggggtttcttttggggatgatgaaaatgctcaaaatttagattatggtgatggctattcaactttgtaaatatactttaaaaacattgattcttaccactgagtttaaacaaccaaaaaaaaatcccaaggtgcattgaattgtgtacttcaaatgggtgaaccttaataatatgtaaattatatcccagtaaaggtgttaaaaaatagtactttaaaggaatctatggtagttttgaaaataaggcagttttccatactttgttaaactctggagaagatgacactttactactggtacctgctagagtaagacttatctagtattaacaaaattagggtttattaatggtataggatgatccaggtaatgggggaaaaaaaccgagcatcctgttatctaatgtactatccagtaaactactctagctttttttcatgaactttttctaaaggctttctagggcctcgtcttggtttgaaagttcacagctacccttcagaaaagaaaacaaaaatccatggagtaggcagatacaagtactcatgtgagcataatttactttgattttttaagttgtgttattctagccctcagcctgttccctgcctgggctctcctagtgcccagtaacactgattcaagaggttgcatttagctgggcacagtggctgatgcctgcaatcccagcactttgggaggccaagttgggcagatcacctgaggtcaggagttcaagaccagcatgtccaacatggtgaaatcctatctctactaaaaatacaaaaattagccaggcatggtggcagatgcctgtaatctcagctacttgagaagctaaggtagtagaatcacttgtacctgggaggcagaggttgcggtgagccaagattgtgccactgcactccagcctgggccataaagcaagactccgtctcaaaaaaaaaaaaaaaaaaattgggtgagagggaggaattgaggaggataccaagggttgggcctgaacaaatggaagcataattatatgtagaaatttctatgagctactcttctagaatagatgactcaataataccctgcttgccatctacgttttctgtccttaattatttccagttctatttcatataatgcctatttcaggccttaacccttcagtaaaggaggtttggtttctataccctaggacagtttcattgagaataaattttgttaggctacctatgtattccctactgtgcagactacagtacagtactagcagaattcttaggctgttactagaatatgatgatgaatgcccgggtggtcatctgtctcccacccggtagagttggcttcaggattgagatacacgtggccctggaggagacgtttcttcccgtcatgctgcagaatgagaacatttccatgttttcgtcattgtctgctgctgcctttaccacctctgtggctcctccctattcaccttgttcacatcttaactcatctgtgccctgttgtgaagcttacacaatatgtaaacaaaactctaccctgttggacaaatggaacacttgtttccttgttgtagttacctgataggttccttagctcattatattcaggatctagatctgtagctcttttcctcttttgctgttctcagaggccactttttttttttttaatgccgaaaggaggattttgtttgttttacatttttttcttctttttgatgatttctgcgttctaagaaccaacccttggatggtttctgattctagaggcaggctttcaaagtagcttaaacctcttaaaaaacatctgtatctagtggtctgaggcttgtttgattctgggatacttaaggtcccccagtaatattggtgtttgttcccctttttagcatgagtctgctttgccctgggatgaccatagaccacaagttacctggcggggggatggacagttttttgctgtgagtgttgtttgcccagaaacaggtatggaaatatattgcagttaaacaacaataaaaaatttttatcttattaaaattaaggaaaattttctttcttttgctttgagtagggtattaattatacatatgaggcaaggatgtgctgctttaaatgtgaaatgaggttagagttaagaattagaagagtcctttgaggccatttggtccatcctcctacctggtggacacaaatttgtaacaaaattaatctaattggctatgtaaaaccatggcagtttttatttgtaaggaaggtgtttgaatagttctgaattgacaacttttatcataatgttttaagtgtgtatgtgtgtttgactccactcccgcacaggggctcggaaggtcagagtgtggaaccgagagtttgctttgcagtcaaccagtgagcctgtggcaggactgggaccagccctggcttggaagtgagtgggagaagaaaccttagagaaattcttggaaccagagtagaggtggtggtacacatggatacagatgatacagatgtttgtgtaacacaaaaggatttttacgtttcttcatttggttataaggctgtatctatctttgtttcttcttttttttttttcttattccctgaagtctgaattcaactcgaatagtagattttacgcttcttcacagatttcattgttccaaggccgcatatattttgcattcctaactcttaaaaggctgtggttttaaggcagggtatatatgaagccattgtacagagcagaaaatggtgtttagaagggaaggcccagtttgcaaggctctgtggggcaaatggtgcttttgtggaaattagggaaagagcctccttccttggcacaaaattcctacagcagaggatctgcttgccaaggagcatgcaggctggattcagaccctgctctttccttccattctcctccttggcccagtacccttgtgcaggttacaatttgcctgtcatatgtggctgcctgattttagatagaagatgtatctcctctgtttcggtgatatctgttgtatgtagacctcttgtttcccaccagtatctgaatggtattatatgatagagcagaagagaaatgtatttgaattaaaaccctagagacaaatatgaataagatgaggcaattaagatgttttcaacatttggtgaagtcttaaaaaagacctactggagcatagaatatttgctgaagttgtataatggaaggagaaatagattttgatttttaggacattatacctggaatggtttagataacttattatttttaaagtcatccaaatgcaatgtaaatatgtaaggttttgtgggcaaatggagcctctgtgtaaaacaggaaaaggcactctttcctctgggcaagtacagtcccacagtgggatgaaccgctcgccgagagacaagggacacatgggatttaaaacttccttggataaagatattcattaattcgttcattcattcattcatgtttgctggaaaaaaaactcttctggattttatctattctttagttaggtgagctttcgatattgtaacactctgagtttgctttaagaccctcaggcagtttgattgcatctacacaagataaacccaaccagcaggatattgtgttttttgagaaaaatggactccttcatggacactttacacttcccttccttaaagatgaggttaaggtaagtgcctgagtttgtttcaccctcgaatgtagaggactttccatagctatagagggaattttttttttttttttttgagatggagtttcattcttgttgcccaggttggagtgcgatagtgcaatctcggttcactgcaacctccgcctcctaggttcaagtgattctcctgcctcagcctcccgagtagctgggattacaggcttgcgccaccacagccagctaattttgtatttttagtagagacggggtttctccgtgttggtcaggctggtctcaaacccctgacctcaggtgatccacccgcctctgcctcccaaagtgctgggattacaggcgtgagccaccacgcctggcctatagaggggatttatatttgatatggatatataaatagtagctttagagtaaatagtaataaaaatggtggcttcctagaactgatttttatttaataaaatattgtttttccagtgattttgcaaataatagcatttgtcccccaccttagataaaacagaagtaggaaataaaaatgctagtttttattgtttattttgacaaaagcataatttttccagtaatgaagatgtttttcatttataacatttaaatcttaagtggtttgtataccattaagattcttgctgaagtgagaacacatcaaatggtatctctgtgtaaaattttaaacatcctaagttgagagacgagtttaatgaactcccatgtaactattactcactttcagtagataccaacattttgcaaaactattttcatcggtccgcaactctttggcctatacatatatatacttacatatatttttatttcctggagttttaattctagaaatcatattttcaatatttatttataacagttaaggacatttttctttacataaccataattctattattacatcttatctctgtgttgtctaacacccagtccatattccagtttctctgattgtctaaaaatgtcaccttgtatttggttaagtttcttaagtctcttttaatctttaagcataatgtatttcttttttttaagtcctctacataataatgacatattttacagatttgtttaatgcctctgtaggttagtgatttacagctagggatgagctcaggtagtgggattatttgatttgagagaggaaatacagctattataaagatttggaagtaaatccataactgaaagccaatgacagatcttttttcccttctaggtaaatgacttgctctggaatgcagattcctctgtgcttgcagtctggctggaagaccttcagagagaagaaagctccattccgaaaacctgtggtaagacagctgtagtaccccagccttctgccccataaaacgtagttgaaagtagacaggtatgggatttccttcatcccttctacttagtcccttagtagaatcaaagatgctgaagtgggtaggtggaaatgggggtggttaggttttgattgattgtggatttcagtcatgtattggttggggttctctagagaaacaaataatacatatatataattcgtccctcagtattctcgggggattagttctaggattgcccatggacgccaaaatccacacatggtcaagtcctgcagtcaaccctgcagaacactcagatatgaaaagtcagccttttgtatacttgggttttgcattcctcaagtaccatatttttgatgtgcgtttggttgcgggtatagaatccacaatatgaagggccgactgtattcattgaaaaaaatacgaatataaatggacctgtgtagttcaagcctgtgttgttcaagggtcagctgtacttacatagagagacggtgagagagggaatagggtggggcgggagggagagagagtaatagagtgtggatagatttactttaaaagattagctaatgtaggggatggcaagtttgaaatttgtgggggcaggttggcaggctggaaattcaggtaagaattgatgttgctgtcttgagtatgaaatctgtagggcaggctggaaacttagggaggatttctgttacagccttaaggcagaatttcttcttttctgcgaagcctcagtttttgcttttaaggtcttcagctgaatgaatgggaccttcccacattatggggaataatctgctttccttatagtcagccgattataaatattaatcacatctacagaataccttcacagcaacatctggagtttagcagatagctgggtgccatagcctagccaacttgacacaataaaattaactgttgtaagtcatcacgtgctttccctagtgcatggtattaccacagaaaaaacactaaccaaaggaattctgtggacgtgaaagaagatttagattaagcgtaaaagtaagaatatttttatagcttttaaaatgtataagtgtgtggttttaagtattaaataatacttgaaaatgttagaaaataagatgagaaaaaaatctcatagttctaccacttcgtaataatcactattcaaattttcttgtcttctaggtttttcatgtatatatctcagtatagctatcatcttgtttttgttaaaagtgtagtaggtatgggccaggtgcggtggctcatgcactttgggggcccagcactttgggaggccgaggcgggcggatcacgaggtcaggagatcgagaccgtcctggctaacacggtgtaaccccatctctactaaaaatacaaaaaattagctgggcgtggtggcaggcgcctgtagtcccagctactcaggaggctgaggcaggagaatggtgtgaacctggaggaggcggagcttgcagtgaatggagatcgtgccactgcactccagccttggcgacagagtgagactgtctcaaaacaaaacaaaaaaaagtgtaggtgtgatacatctgcatcattttaaattgctgtataatactcgtttattctcgttcattaaatctcatgctgttagacatttacagttttgtcatttctcattattgtaaacagcaatgcatggtacatttttgttcataaatctttttacttgattattttctaagtagctttcaaactctttaatcagtagaaccccccccctttttttttttttttggagacggagtctctctctttcccccaggctggagtgcagtggcccgatctcggtcactgcaagctctgcctcccgggttcactccattttcctgcctcagcttcccgagtagctgggtctacaggcgcccgccaccaagcctggctaattttttgtatttttggtagaggcagggtttcaccgcgttagccaggatggtctcgatctccatctcgtgatctgcccgtctcggcctcccaaagtgctgggattacaggcgtgagccaccgtgcccggcctcagtagaacccttttaactgcaatgttaagaaactcattattcattcaacacaatagttcttaaccctggccacacctttagaaaaaaaatgatattcaggcttcatctaagagttcagttcagtgtgttggaatggagattatacgtaagtatttaattaaaaaccaaaagcccccaagtgattttaaacagccgcagttgagaaccaccgattaaccagtgtgtcaagggatggcactgtgatatgctgagcataaaaatattgcacaggatgaaaccctgtctctactaaaaatgcaaaaattagtccggcgtggtggtgcgcgcctgtagtcctagctactcgggaggctgagacaagggaatcgcttgaactgggaggcagaggttgccgtgagccgagattgagccactgcactccagcatgggtgacagagtgagactccatctcaaaaacatgtatatatatatatacacacacacacacattgcacaagaacagccacaacatctgtgctcacagaacatcagcatgtggtctaacttcaaagtgttgtaataatgcggtttgagactaggttatgtttgctgtgatcactaagttaagcattagtgagcaaggagattgagaaaatccttaatataaataatatttcttaatataactataattcctaatataactaaggtcttaatttatatgtcatctgtttagtaaaggttggttttggcatgattaagtcttgcttgcttaatagatgttggaaggataatttcatgcttatcttctttggacagctgaatcaggattaatacccagatagccttgaacataagtgcttgcaaagcacctgaaagaaaataagcatcttaagcccaatacaacacaatgatgctagtctagatcttggattaagtgttttaatacttttactctaattgccaagttatcttcttcctaaatcttcatgagaaaacccactaaaagaatgctttttcctggtagccttccattgtgatcataaagtttggaagtaaagttgaaaataaacatgtgggccaggcacggtggctcaggcctgtaatctcagcactttgggaggccgaggcaggcggatcacaaggtcaggagatcaagaccatcctggctaacacggtgaaaccatgtttctactaaaaatacaaaaaaaaaaaattagccgggtgtggtggtgggcgcctgtagtcctagctactcgagaggctgaggcaggagaatggcatgaacccgggagatggagcttgcagtgagccgagattgcgccactgcactccagcctggccggcagagcgagactctgtgtcaataaaaaaaaaaaaaaaacgaaaataaacatatgaataaaagttaaaaatagaaaaaaaacaagaaaataaacatatatttctgaccttattgattcttgatattttatctgcatggaaagctattttttggcagttattattgttcttattttagagacgaggctgagcaggaagggtcctttgaaaaagaaaagattgcccttgaacccctctggcaagtgggatgaagtctgcttcccagcctctaacggccttcttttcattttcccttgcagttcagctctggactgttggaaactatcactggtatctcaagcaaagtttatccttcagcacctgtgggaagagcaagattgtgtctctgatgtgggaccctgtgaccccataccggctgcatgttctctgtcagggctggcattacctcgcctatgattggcactggacgactgaccggagcgtgggagataattcaagtgacttgtccaatgtggctgtcattgatggaagtaagctcctgggaagtgtgtccatgagcctgcaaggggtcctgagcctagggcctgcagatgtggtggtttgactggaacagtggggaatctttatttgttttggctgtttgggttacttgtttttttattgaatgggatataaggtggggtatgttctctcctgagaaccattgtcccccctcccccaccagtttcctgttatactgcatctgtggccttcacacgtttacttgcctggcctttgaagacactgaaaactttgactctaggtagagaggatgacaacagtacagtcttgtgggattgggtgtgttagctttatctgtttgccctgacacagatttataattgacccttataccaccccacttgtgttgctttgtttcctgatacaaatgcttgctgatatatacctctccagtatgttcagttcatgcataaacgtttgcctaatatgaagattaggtttatattttataatgaggtagaaggtttttttagggggtggggtgggaagggcaagactgaagagtgaagtagtcaccttaatgaatagtttcattgctgatatgaaagggagcactggcttctaagattgtaatgtgaggtggatattaattcatattctgtgtaatattctacataatactgattttatagtcatgtattctatatagagaacttaatcagatctgcgttattaccaaatccacacataggaaagtgctttaaggattttgaaagtattaattcccttggtttagtgtggcttggttgcaggcccaggtttaaagctagaggtctgacctcttggcctttttgccttagtccctggcacctgaaactccaggtactgagatggactcccctaggcctagaggtgacaatagccaattatggacagaacccatgacatttccccatcccacactgtttttagacttgttcctgagaaaaacattgaaagttatttttttgtgaattgccattattgtttagatatactgtgatgttcagatggcttatcttacaaattgaatatccctaggtctaatcctcttctttctttttcactgcagacagggtgttggtgacagtcttccggcagactgtggttccgcctcccatgtgcacctaccaactgctgttcccacaccctgtgaatcaagtcacattcttagcacaccctcaaaagagtaatgaccttgctgttctagatgccagtaaccagatttctgtttataaatgtggtatgttataaaacttttgccaagatgttctgaatcaagtcccttctactcctacataaaagcaaattatagtttggtgttgccataggtctagtgtttctcaaaatttttaagtctgcagttgatatcattatcattatgatatttaattgccttgggtttttgttttttttttttttaatcctatactggtttgtacgagccattccttttcccttactgacttgaagagtcagttatttaagaataacattggactctggaaataacatagtatgttatacattgttaacatgttttactcttttcatagcctttacacatattttcagttgatctcatccctcctaggagctgtgtcagagatggggttttcctcttttgtagatgagggaacacagtgtcagaggttttgtaatttgtttgaacaagaatggacaaggacctcaacacaggtgttctagctcctaatccacttgtcctgccacagccccattgctgtcagttcttcattactttcctgatgtgctggagaatctgaaatttgtttttacttgtgagttctgtggttatgtcataaattctgctggcatatggcagtgttagccttgttttcaaatatcttttgaattctcagaaaaagcctagatagttgccaagagagaataatcaaaattaattaatttaaatgggaagtccttactttcatatcagcttttctgttaagtcagcagcccactgtgtacatggatcctatctggatgtatcaccagtttctctgattatagtttcagtgtgtaaaatgctgttacagtcctccttaaacttttcaaaatagctttaaaaaaaagtgcaaatatgttcattgtcaaggcaaaaagaatcagatgtaagcttttgtgggacttaactgtatgatgctaatgagtttatatgtcactttatgatgtatggtatgttttgttctgcattcacttaaaaaatagctttatatcattcatctatttaaagtgtacaattcaatggtttatatgtgtgtgtatgaatatatatacatatgtatatgtatatatatgtatattcacagagttgtacagccatcaccacgatcaattttaggacgtttttatctcctcagaatgaaaccctgtaccaccctgcattcattttacttgagagaaaactccctgtgatgagataggacaggttgagagctccacttttgaaagattgttcggcatcaatatgtggggttggccataggtcaggggcacctggaggcagagattctagttaggagaagctgttgtcaagtgtccaggcaggagctagcaagagcttgagccagagcagtgttcatagaaatggaaagaagagaaagatcataacaaatccatgaagtaaaaaccctgagaagttaaagaacccactggggagagtttggatataagagaatctggaaaaagagatcttggactggaacaggtcagggctccgtgcccaagtggaagggaaattaagaacttggagtcaagtggtagacatttgagtggtgtggagacaagttcgttgccaaagttttcaaagatggtgtttgatgcatcctgagtatcactcctttttccccctcattgcttcttgattgtttattatatgccaggcttttttctagtacttggcttgttgtactagaaaactagttgtactttgtctacaacttgttgttctaggtgtagacaaaagatatcaattaaatatgatctatcagatggcaagtgctgtggagaaaaattaagcaaaataaggggtagggagagcttaaggataagggtttacagggggaaggtgtctttcctatttagtgtgatcccaaaggcctctctgtgaaggtgacattgaagcagagacctggtgagaatcacagtgggagccacgcagacatctggggtaagagcgtcccaagcattctatgcttgaaggcaaagaagaaaaaagaaagagcgttccaagcagagtaaaaagcaaccaccgaagtgcctgttgtgtttaggaaatagccaggaggccagggtggctgcagcagagcaaaggaggggaaggtggtgggtgagttcagagtggtgatgggaatctgctcttgtagggccttgcggcttttactccgagtgagataggagccaccagagggcttagaacagaggagtgcagtgttctggctgaattttttaaaggcttgcattggctgctgtgcagtgaataaactggatgaagaatagaaagaaaatgtcttttaagcaggtgcttaggactttggagaatttgaggatattgagaggtggttgaagacagtggaggaaattgtccacagcactgggctgagagggtagccccttcacctggtcttgctgagatgtggcctttgtcagggaagattatgactgatgtgttcttaagaggaaagcagagattttaaggaggttgagatgtgattattttctagattgctgtttgccttctagaactcattaattgcagacaccatccccttagtattaggtgaaatcttataatttacgatgataatatttgcatttttgttttccaggtgattgtccaagtgctgaccctacagtgaaactgggagctgtgggtggaagtggatttaaagtttgccttagaactcctcatttggaaaagagatacaagtaggttcttaattatcttgggcttctgggaacagaatcagccagcatgcagtcctaaattcagccatctgataacagttctatgcctgttgctgagtggaacaagaaataaagacaacacccaggccctgactttcggatctgattggagaagccagtcatgtagtttgtctgaatgccatataatttgataggtagcaggagagcatgagttgtaagccagcctaggacctactcccaatagcgcttggttctccaggaaaaatcatgtgggaaagatggagatgacaatgataaggcggagctgcattctcttacataaatggggatgtatgggttgttaacatggatgacctaatgcagcctctgtctttgctccatcccagaatctagaacttctgggtgctgtgctttgaggctcctgggatggaaatcagaatgcattcttccattgaaacagtattgtaaacaattggatgttattgaatacctcaggtacactataggcatttgcaaaatgacctagaaaccaaattataatgccacatctgtgagagaacttttttaaaaagtaccacttattgagtacttacagattaaaaaaacaaagtgtagaggttaggtaacttacccaaggtcatggacctggtaactagagaatttagggtttgattctattctgtttgataagtccatgttcttcattactaaactactctgcctccagggaacatttattgttagattaatagaaataattaactgagtacaacaaatagcagaatttaataaataatgtttcttaaatatatgtgatatatttaataaatacagcagaagtgttcaacctctgtatgattttgaggctgcctgtataatgcttagtagtttttaaagagcatttacatgcattatttcacttcatagacttgaaaccactagagtagagatagaggacaaattagaaagtatgaggcagtttagaatatagtttcatttaaaaaaaattgatggggataatgccaattcgtctgagatttcacagaagacatgagtactcatcgtgatcttggggaagggataggtttggggttggcaaagaattgggaacattgggtctggtggggaagaaagtgtcagtgaaaaccagaggtgggactgatcctccatgggatactctatgtgaatgcaatggagagcctgagtccggggagagatgtttgaggaggaagatcaggctagtgaccaacttcttcagtgggagctgcggatttgccacctgatctaaaaggcaggaagtagccattgtcggttcctacgtgaggtgacaagaacagtgcgctggtcaggtgtataaatgctaccaaagaatgcattagagacatggagaccatctctcaagctagtcagtcagtttaatgtgaggtgcttaggaaaggacccattctactgcaagtgacatacctgccagagcctggtttgaatgctggtaagtcatggcagtggaaaagctctggggttcattagtgtagggactagggctggtaattttcttgtgtagtcagtttcctcaagtgttctcttcaaatttaaagatttcagggtatgagaaatttagggaaaatataaaaacgtattcttaagccagacaaagattaattttagattttgtagtatttggtagtatctcaggttttgtccctccaaataattaggagtggactgtatacaagatgcttcagtcttccttcatccaggaacgtctcagtggtttttaagttttattcatgtcttggatattcttcaatatttacaatagaatccagtttgagaataatgaagatcaagatgtaaacccgctgaaactaggccttctcacttggattgaagaagacgtcttcctggctgtaagccacagtgagttcagcccccggtctgtcattcaccatttgactgcagcttcttctgagatggatgaagagcatggacagctcaatgtcaggtattgcagtttttccctgtactccacatgttaagcaaatggagttaggtttttgtcttttatgagcatacaacttttgacttctattgatcaaggttgaggagcagtagctttcttgttagacacacttaacaagaaggttaagtctagttatgagccatgtcaaaataacagaccaaaaatatatcaaaaagtggtgaaaaataggataaatattagtagatgaagcaactttttaaagatatgttaaatattttaatttagcatctacccacatttttccagcgtgattgttatatgttataattgattttaataactgtcaagcataattagagtggctaattctcatgggctaatgtgatgggaagaaattttgtataaatgcagtcatgcgcatatatgtgtgtgtgtgtgtgtgtgtgtgtgtgtatacataccttttctatgtttagatacacaaatacttgacatggtattacaattgcctgtagtattctgtaaagtaacatgctgtccaggtttgtagcctggtagcaataggccataccccataggctaggggtgtagtaggctacaccacctaggtttgtgtaagtactctatgatgtttgcacaatgatgaaatcacctaacaacacatttctcagacgtatccccatcgttaaatgatgcataattgcacatatatgctttgttttgatgtggtgacttcaaaatgcttcttccagcctcctcttctatatatcctattttgtacctgactacatttaccattagaaagtctctattcttctttgctgaaatttcactgttctctgggcctgagttttgttttgattcctgactatatcttcattatgtaacaggtttcagttaatgaatgctcttctgtgtaatgtaagccctgttgtatagttgatagcattttctagccagttcccagaactccttgtttccagtgtcaatacttggcacctttgtccactgacactaatccccagattaatttgtaattaaagccctactggtgagatttctgagaaacgttgttgcaaaattaggaacctttcctttatatatatacattacataaatttatagacataaaacattttaatgcagtcatttgctgctactctttgactcatagtctttcgtgatattttgaaaaagccttttgttaacatgtctaaatgcagaatatgttctagaaatatgtagcacttaaagtaagccattagattaccttttgaaaagcggagcaatttactaagtttctacttcttcagatttgaaattcttcatcattagcttgtagaggcaaaagcttgatgcagtcatctcatttgctgtaaaggaaatgagaagtcatttacagtatatttctactgctttgacttttatttctcaaaaagactgttttgttcatataaaatattaatgcttttgaggactacaaagtccctcgatttagtttacatttactttagcttatactttgtaaaaaatactcttctaaatgctttgtctgttttagcttacttatttctcataatacctctgtaaagtatatgccatttgcaccatcattttacagatgagacaactaagacatggagcagttaggtaacttgcctgagatcatgcaggtggagccaggatcaaatcccagcgagtctagctccagagtttgttctcttcttgacagataatttatcctcacaaaatttgaagcatttgtagaggaattccctattgttataatgtttagtttttttgtagattggttaaaaactttgaattaaatgttagcattaacatcatttgcttttatcactacttctttgtctcttttttctttttttaatcactacctcttcctcctcttttgagaaattctgcttccgtggctatggtccaagctacttgagaaggtgaggtgggaggatcacttgagcctaggaggttgagattgcggtgagctgtgattgtgtcaactgcatttcaacctgggcaacagagcaagacactgtccaaaaaaaaaaaaaaaaatagtgaaattttacttcgctccattgactcagggaaaaaatgtaatggtgataacaaattcccttcatctcattagtgaaaatccacaattttccatcaatcgatatgatagtgatagagatattgagtgtgctcattttcctacagaccagctgctttaactattttaagcagacagaaatgatattggtaccatccatgtctaatgaaggcaatactttgtaataagttgcagtaagttgtggccagaagaggaatgatgacttcacagtgtaaacaactaccttattgggtttgtggaaaatggtgtcatgtagcagatgtggctttatctgggctttggtttggagtagttttatctattcatctaaccgtctgtctctaagtgtataagtgtgtgtgtgtgtgtgtgtatagtattgggtgtgtatatatgtattttgtctacattgtattgaagtaggtagtgcagcatcaaaaggaaattgttgattttcaaaatcagtgaaatgtcactatttttgagaaaaatggtctgtttacactcccttctccttttttttgtcagttcatctgcagcggtggatggggtcataatcagtctatgttgcaattccaagaccaagtcagtagtattacagctggctgatggccagatatttaagtacctttggggtgagtatcaaggtgttaggaaagcatgttatgacttacatagatgcttagttcttaagaacatgtacttgtatcttgtcagttcaatattgattgtcaggtcttttaactaccctggaaaaccctaagctttagagtggaattggcaagtgtattctactcctgtttcctcttttaatgaactaacgtactcttaaaaaagtgattgatgactatcgcagggacaaaaaacgaaacaccgcatgttctcactcataggtgggaactgaacagtgagaacacttggacacaggaaggagaacatcacacacttgggcctgtcgtggggtgggggagggtggagggatagcattaggagatatacctaatgtaaatgacaagttaatgggtgcagcacaccaacatggcacatgtatacatatgtaacaaacctgcacattgtgcacatgtaccctagaacttaaagtataataaaaatatatatataaataaataatgccagcattagagaaaaaaagtgattgaaattgcatgttaagtgttttagcaaatgttgatgttgatggttttttgcaaagagcgcatcagctatttgtgaactagatctgtgaatcttgcagagtcaccttctctggctattaaaccatggaagaactctggtggatttcctgttcggtttccttatccatgcacccagaccgaattggccatgattggagaagaggtaggtgaacacggagcaggaaatttacttaaagtagttacccagggactgatggcattaagtagaaagagcgtgggctttggaggtggacttgggtctccactaaatgcctagacaatagtgggaaatgatctcactttcataagccacaccttattcatctataaaatgggaaaatcagtatctgtctatcagggttcagaagactaaatgagataatatatgtgattagcaaccttttatccctagttgtacaaatcattcaaagttaattttatttagtaggggaaacagaaatgtgatcttgagaatagttttagtagatttttattcaacacatactagaatgcctataattgtggtggatggtagaatgcagtggctggaaaacaaaaccgcttgactaattcctgctcttctggaacttgtgatctattaatttcaatgtaatgattccctttgttgggagtgtgatggaaatggacagagtatactggtagagaatactgagatgtttgaggggtaatttgaggatggtggctatgagaatgggagtcctgcatctggtggtccaggaaggcctctcggaggcagtgatgtgtgtgctgagatgtgaagaaaaagaaggctctgtctccaggcagaaggaacaacaaactccttgagcttagcaagagctcatcttattcaagggactggatggaagtattgtggctggagctcagtgacagtcataggagggaatttgggttctttaattgaacaaagattagaaacttcttgtgatttttaataacagagtaatgtgttctgcttcatggtttggacagtgattctggctgcccagaagagacttgattggagagtgacgagactggaatatgggatcaacaccggttgagtggagttagtgaggggaaaaaggagatgggtttgagatatgtgtaggagatggagatgtcagggctcactgatggattggatggcttcacattccgttttgcactggaccagccacgtcttaggtatctatctttagtcctgattacaggaacttaggtgtgaaatcatagggtggtagaactatgtgatagaaaaggtaggtttaactgatttgagatagaattgcttgtgatttcagttttatttctttgcaggaatgtgtccttggtctgactgacaggtgtcgctttttcatcaatgacattgaggtatcaaggcttggtttggtgttggatccttttcacagtgttagctccgagtaatctagctagctttcacccatgcctctctggccttctcttgcaggttgcgtcaaatatcacgtcatttgcagtatatgatgagtttttattgttgacaacccattcccatacctgccagtgtttttgcctgagggatgcttcatttaaaagtaagttttcaatgtataaaacagaaatggtcccttctccaatgtcttttggagtcttgatgactttttgaattcttcatttattttggctttttatcaaggagtcctaggctggagaaaatctttagagttattttacttagaccctaatctcaacataatatctcagttaaatcattctgcactttagtaaagacatccaaggaagggagttccttccttaagcagcacattctaaagttaaaaacttttcaggaaattttattatgtaactgatctaatattttatttggaattactatgtagatccccaatgttttaccttctgtgtagtcttttcccactgtgcccaccctccactgtacatctgcgctccatctagtggtttgtaggatattggctgcattttgtcttctgttccatgccctatctatctctgtgtgtgtggcgtgtatgtgtgtgtggcgtgtatgtgtgtgtggcgtgtatgtgtgtgtggcgtgtatgtgtgtgtggcgtgtatgtgtgtgtggcgtgtatgtgtgtgtggcgtgtatgtgtgtgtggcgtgtatgtgtgtgtgtgttccttattctaaaaagccaacttattttctttgcttccaacttggaaatagggaatctttctttcattgatatgattatagtacactgataatgctaagaaatagagaagttgccccaattcttaactgtgtttctccacatcatttgagaagctgtgtatgtgaatgtgcatgagggctctgtaagagagagggcaagttccagggatgagcgtgttcatcagcagggctgatagtcttgaggttcagtgggagagctaaggcacatggttgttatttgttctcttctatttcacataatgtgtgcggtttcaattgcagttaatggagagtggcttgttgtgataattaaggcttattagttaatggtgtgtttagcattacaggccggcctgagcagcaatcatgtgtcccatggggaagttctgcggaaagtggagaggggttcacggattgtcactgttgtgccccaggacacaaagcttgtattacaggtaagctggtttttcagacaagatagatagtctgattgtcattcagccaagtaccaagcataattcttgcaggttgtattttaggctttcttattctttgtatcgtttattgtaaacctttccttgatagttttctgttagctttattcaaaggagtgttgatacaggctgtgaccataaggctcaaagcgaaacttttcttgaaagtcaagataaatatagagaacaacaagattctgctaaaagtgtgctgattttagagagttgtggtaattctctgtgaagagttaggtaaaatggtgtatcctggctatttaaatgttttctacttaattaaaaatgttactgctttaatttatttaagatgccaaggggaaacttagaagttgttcatcatcgagccctggttttagctcagattcggaagtggttggacaagtaagtgccattgtactgtttgcgactagttagcttgtgatttatgtgtgaagacaataagtattttattacaatttcgagaacttaaaattatgaaaagccctcattacctatatcatcaatcagattcttagaggctcttttttttttttttaacttttttactttaatgcagtattttgtagtggagattcctagcagaaagaatcgtgacactcatcatataaaggagggcttctcttaacctgagggaacacatgtgggttttaggtggcctgtgaacccagggagattgtacacaccaaaccttgtctttgtgtatttattcaagtagaaagcccacagctttcaatagatttacagcggggcctatgacccagaaaagcctgagctactcttgtgaaggaaatgactgattttctgaacctatttggaggaaactttgtattggaaagatctatactaatgttttgtttaaaaagtagacctgaattccatgatgattttctttgttttttttttgagacagagtcttgctctgtcacccaggctggagtacagtggcgcaatctcggcttactgcaacctctgccttctgggttcaagcaatcctcccacttcagcctcccgcatagctaggattacaggtgtgcaccacgcctggctaatttttttttttgtattttcagtagagacagggtttcaccatgttggccaggctggtctcaaactcctgacctcaagtgttctgcccacctcggcctcccaaagtgctaggattacaggtgtgaaccaccgtgcccgggcttctgtaatgattttctgttgtatgtatgtgaagatgtagttctcagacagtcatgatgactaaattacaccttttaagaaggtaaatgaatgtggtacctgatttttttattctgtaatttcagagtagaaatccagtgatagtagcttggcattggggctgtaatctgattataactggtttgtatcataatgaaaatatgctgggcccatggagctcagtttttgtgaatatcttttctattctttctctgtcttctcacagacttatgtttaaagaggcatttgaatgcatgagaaagctgagaatcaatctcaatctgatttatgatcataaccctaaggtaactttctaagctgtcatttactctagcttactttgtacttaaactaatatgatctgaacgaagatgttttgtcctttttttggtaggtgtttcttggaaatgtggaaaccttcattaaacagatagattctgtgaatcatattaacttgttttttacagaattgaagtaagtattttgaataattcatgtgtatcttttccatagttttctctcttcttgttaaggaaatcaagcataaatagctagagaagaaaaattccttactgttcatttttaaaaattgctataactcttagatgccagttggttttttgctcttttccgttctttttaaaacagcctgtttaaaactatgtccttaaaacatgtcattcagaattattatttcacttgatttttaggtatacatataaaactacttgtttttcctaggagactgaaatcaaatggcatctttctctctgatgatctttcccctcaactttttaatgaaacactttcaaaatagagaaaagttgagagaattgtccagtaagcaacctatatataccccacctggattcgccagtttatatttttctgtatacacattctcattctctataatctgtccatccatcattcatcttgtttgtagacaaattgctaagtgagttgtagacatcagtccactctaccacctgtacttctccttgtatatcattaactagagggcattctttgtgtatgggttggttttgttgtgttttttcaggtcatatttatctacagtgaaatgtccaaatcttaagtgtgccacttagtgagttttggcaaatgtacacttcatgtaacctgaacctctgtcaagttagagggcatttactccttttcagaaagctgcttcagattcctttcaatcagtccctgtcccattccccaggcaactactcttctgaattttttaccataaatcagttttgcctgttcaagaacttcacctaaatggaagcatacagtattactcttctgcataaagctgttttcattcagcatattgtcttgagattcatctgtgtttttatatgtatcactagttcattctttttttattggtcagtagtatgccgttgtgtaaatacaccactatttgcttattcattcccctgttgctggacatgtggattgtactaccctgtttggggctaatgtgactaaaacatctacaaacatttgtataagtcttttgtggacatgttttatttctcaatatttttataattcaactcttttccaaaagtcatttttatttatcatcatcagcatgccaggtgtatgttagtaatttgatcgctgggctacatgttctgttgatgaccattccatacacacctgttcttagagaagaagatgtcacgaagaccatgtaccctgcaccagttaccagcagtgtctacctgtccagggatcctgacgggaataaaatagaccttgtctgcgatgctatgagagcagtcatggagagcataaatcctcataagtatgtatgctgtcaccaggtggcatcctttgaaaaaccgaagtgtgtagttgtccttgtccagcctacttacctttctcattctggtgttcttcacttattacctcagatactgcctatccatacttacatctcatgtaaagaagacaaccccagaactggaaattgtactgcaaaaagtacacgagcttcaaggtagagatccgctcacagagaaagtgcttaaggtggccgtgactgctactagtcttctgcaggtgacaatcaccatgtcattgccacaccacagatttaacatgtgactttttagttgccattttaagacccttgtcagtttttttcagtgctgccctctaaagcatatataaaagtatcagaagtatatattcttctgatgtccagttctattgagaaaaatttattgtctttttggttatgttgttaggtctgtggattttttccccaaatgattgtgttctgttttgttttctaaacactgttaggaaatgctccctctgatcctgatgctgtgagtgctgaagaggccttgaaatatttgctgcatctggtagatgttaatgaattatatgatcattctcttggcacctatgactttgatttggtcctcatggtagctgagaagtcacagaaggtatgtggagttcttacttttatgccatttggttcttgtttatataatgatagtgtgaaaccctgcttctggtagtgcagtagcttttctgctatcactctgtgagtgcagggctggagacagatctgtgagtttctagggcccacattcctaagcccctgtgcttatgaaagtgttttgattgtgaggttgaagaagtgaagtaaaattgcatggcttttttttgtttctttttttttgagacggagtctcactcagtcgcccaggctggagtgcagtggtgcgatctcggcttactgcaagttccacctcccgtgttcacgccattctcctgcctcagcctctctagtagctgggactacaggtgcccatcaccacgcccggctaattttttgtatttttagtagagacagggtttcactgtgttagccaggatggtctccatctcctgacctcgtgatccgcctacctcagtctcccaaagtgctggaattacaggtgtgggccaccatgtgcggcctaaaattacatggttatttttaagatgatgggcatatgtgtgagctaatttcttctcttataaaggaaatgtaacaagtggttcatgttccactccggttctttctcacatggctcttttttctagtggagggtgggcacatggagcacagaaggctcatggcctcctttcctatgttggtacatttgctatgatcaaaaactttgaacaccactggtatgcatattttttatttatttttttgcagcctcagtctcttccccatgacctctccaaaaatgaaaatcggatccttcatctctctgcttaaaatacttcatgagctcccattgttccgaggatataattcagaagccataatactgcttaaaaacccttccttgacctggcctctgtgtatctttccattctcacttcttggtattgtctttttttcctctgcccatggaggaaagacaatgcttttgtcccccttcccttgcccctcaccaccacatgccttggtgggcagcattacttctgccatccatgggctttgactgcttccaccctcaccattcccctggctaattctcactaatctaggttaaaggatgccaaggtggcctcttcccagtaagccattcatgcttccctccagggactgggtgaggtgaccctcctatatgcttctgttgcacacagtgcctacccctgcagactacagtgtgtctttatctagagtgcggtatttatttatttatttttgagacaaggtcgggctctatcacccgggctggagtgcagtggcaccatcttggctcactgcaacctacgcctcctaggctcaagcaatctcacctcagcttacaggcgtgcaccaccatgcctggctaagttttgaattttttttgttgagacggggtttcgccatgttgcccaggctggtctcaaacttgtgagctgaagcaatccatctgcctcggcctcccagagtgctgggaatgagcacttaattatttgttgtcttgggttttcttcctatgttgttcttacatgtatttatcctgtcagcccagggaaattgcattaaaaacaggaaacacctctccattaggaagaaaaacaatttgcttacagggcatggcatagagctggagatgatagtgccaataaatactaggttggcagggtctcagagttttgtgtccaactcagtataattttatgtttgttttaatgtgatcatttcaggagagcatggaatgtcatgaaaacagcaccaagagcaatgtcttagacttttaggagaaacttagatgcatttgttgaatatcttctagactgaaaccttatttcccttattagcctatgaaataaatgatactgtgagacttagttaaggaagttactattattccaagtgtaacttattaatatccgtatgtgaaagcatttttgccaaagcttgtttgatgttcagctgacccttgcacaacgtgagtttcaactgtgcgagtttgaactgtgtgggtttatctaaatgtggatctctctcaaacacagttggccctttgtgtccacggcttctgcatccacaatcagtgtggatcaaaagtacaatatttgcaggatttgaaacttgcagatacagagggccaacattttgtgtatccaggctccatggggtcaaatgtaggactggggtatgcttggattttggtatccttggggtgtcctggaaccaattccccatagatactgggggacaactgtagtttgattttatatattatataatatgcagttaatatataatacacatttaaaaattatgtagctttgggtttattgctatatgtaaatgctagtttctattcctatatatgaatatcacaagtaataaagttctcattaatcatttttttaggatcccaaagaatatcttccatttcttaatacacttaagaaaatggaaactaattatcagcggtttactatagacaaatacttgaaacgatatgaaaaagccattggccacctcagcaaatgtggtaagtgtggggattagtatgtttatctctacttcagatcttctttggaactaggcaaggtataaattaaactgttagtttagacagtgactgatttcacttcccactcctgaaaactctaacaattatgtatgctcacgttattttgtcctgtgttctgaaaagctgaaggtaatcacttttaatgaactggaggagctccctaggtaagaacgtcaagtagatccttttttggttaagaatgagcacctgtgaagttaacttcagtgtctcagaatcaaaattggttgacagttcttccttctcatgctgtttgcagacatgtcagggaaactctgcttgtctggagagagtgatgaggccacctccccgtgccctgcaagacgcagttttaattgacagtgatggggtgccagttgttcttcccatgctggaacagttgtgattctttactgaggactgatgggggaaaggaagaatcacctggggtgcatgttaagccttcagctgctggcatccttggagaatctgattcaggtggtctgggataggactgaggcgtgcatgtgtctaataagcttcccaggtgatgtcttttcaaggaggctgagaaaacactgggctggaaagctgggactcttaagtaggatgctgatcccaatcagtgctgctcttgcctcagaatctgcagtggtgctcattaaaaattcaaattccaggatcccattcttcagattctctgattatttaggtcttaaaaagttcctcatttattttgtttggtgaccattggtataaatgaagtccattatgcttcccatgtcttaagcctgtctttgtgtgaatctttttcctgcaggacctgagtacttcccagaatgcttaaacttgataaaagataaaaacttgtataacgaagctctgaagttatattcaccaagctcacaacagtaccaggtatgtggtatgtgaaaatgaggctctcctggttttgctttttgctttagtaggaaaggagtgaggatcctaagttcataacaccatccttggcttcaaaatttatcttaaaactaattagcctcaatttgaacttcttatctgggagaatggtcctgacctgttctctgattcctcatctggaataccacagcaccttcctcgtggggttccctgcttctttcccacccctcctctagcccaaccttactgctgtaagtctgattatcctaacaagtacagatctttcccatatatttcagcataaagggaaatttttgtttgcttgaaaaagcatccctttagctttttttatataccacacactttgcttctaagttaaatgtgttatatgatcctcttaacagcctcatagggtgctgtacacaatttgtagatgaggaagcaacttgcctgaggatccagagctacaaagtgctggacctgggatacagagcccaggctgcctgaccaccctgcccatgccattaaccaccactctaccatgccaccagcatcaccattttcagtttgtcctcagacaatatacacatctttctttgatcaagcccctgccagcttctttagcaccagcttctgccactgtccacattcccagttacttgtaggtagttctacagatgtcacatcgtgtgattcctctgtcatttctctacccaccagccttcctttagccccatttgtccatcagaacccttgggttactcctgaatgccattcctggaccaggcgccaaacactgagcccccagagcagcctgccctcgccttggtgattgcatttgtcaaactgctgattagctggtttgtcacctccaccaggctgtgggctccttaagggcagggactccatgttgtattcctctctgaatctctggctaacatccagcctggagaatcgaggatttggccagtggatacctctttgcccttgttttctgttctcttccacactctctctgctctagtcacactggccgtcctgttactcctcagacctgctatacacattcctgctgcatggccatggtgccttctgtgccctctgcctggtgccccctatctcatcacgtggtttattctcctgacagccattagagctcacactccctgagagctgcaaggagactgtcctctgtccctttactcacgtttgccattatgctatagactatattttgtccctaagtccatcctctgttactataagagcagcaacttggtggtggttcttatatggtttttcatttgtttggttttattttttgccttgctgtagtatccatactgcccagaatggtgcatatgtagttaagagtaattatttgttgagtgaataaatggcacatcctcagtaaggttttgaatgaaaaaatgactgtactaactgatcaactgtaagattttcccaggtaattctttcaagggagttccaagtataggaactaaggcagctacactggagctttagagaaatgattgtcatatttcctcctcagtcctaaatctcctcttgtcacaggatatcagcattgcttatggggagcacctgatgcaggagcacatgtatgagccagcggggctcatgtttgcccgttgcggtgcccacgagaaagctctctcagcctttctgacatgtggcaactggaagcaagccctctgtgtggcagcccagcttaactttaccaaagaccagctggtgggcctcggcagaactctggcaggtaagtacaatcatttatatgtttacatctacaaaggttttaaaaaatttatttcttttgtttggtaattttgcaaataaatttagggcagaatactctgagacagtcttgttctcactgataaaaattaatttagaatgctttaaaggataagctactacagcaagagtcccagaatgcagtggcccaatatggaaagaagtttatttctctctcccatagggatttataggcccttccgttgtgtggctctgcaaccttttaggcagatggttgtagctgggttatctccacagctgtggggaaggaaggagagtggggagaagttagaatcatggtaaaacatttacctttaagttggaaatgacctggatggaagttaaactatcaccttctattccatctcggccacgccatgtagctggatgggctgtgccctgtaagaaggtaaagatgaatttttggatgggtccattctgttatagacagtaggttgttggaatagccaggaatgaggtggggaaaataaaaggccaaatgtcgaagcattctgaaagcaaaggcagtttagctgcgtcagggacaagggttgcccgaaccagaggcgaggctggtaccaggggctctagtaccagagtggaggaaagggtaaggacacctatgaaaagagatgagcagaagctctggtcatctcagcagtgcttgaagtaaagcaatgactggtatatttttttccctaacttgtaaatattgttgagatctcaaagaaaaaaataaaaagcagtcctaaaaaaattccaaactctatcctgttaaattttgttaaatttatgtaccagtccttctttgtcatttgcagtattctttttttcttgggattataccagtgtatgggattatcacttttctttttctggttattagcctttcccaaatccctccgtttccatgctggcctctttttacaaatgtcgagaattccttatttcaggccttttagttattcgttcggtctccattgttcctttctgctttagaaatttatgatattggttgtttataccttctatctctgttcttggatctcttctattctttacagctcttagcttgctatttcccatgtcttatgagggagtatttctagtttttctcagatgtttagcaaaagtaggtggggagggcagtggtcaaagatgtttgagaaatgttacacactggagtcactctgtgtgtacatttaacgtaggcagtttacacaagagagcaaaagaaaggtaactatttaaatagtggaggtgattttacctactttttttagtgatatatgcactggagtgagcatgcaatgagagaccggaatctaccagctccttcgaaagccttgggttctctgtgcctctcattgtggtttatctcaattgggctgagagtgattctaggatctaaagacactgcatgactcaaacataagtcagctacctccatctagtgctcaaccaaagaaatagtggtctcttactgttaagggacgaagtggtttagtgagagataccaggtcattttcccatatacatgctttggaagcatctttcaaggctaattttggctgtatatgattttcaattcctgtgctaaatttagattctagctgccatttaagataggactctgtggtgtatatacctattccctcacagaaattcagaaagtacatagtttcatacataataaagacatattaaagaagcacttgagctaaagtatctgtttaactttgtagtcaactgctgcttattgtctctacaggaaagctggttgagcagaggaagcacattgatgcggccatggttttggaagagtgtgcccaggtaaactcaattcctcccttctaaaccccccagtcagcaagaaaggtcttctcaattgtatcttagtgatcatgaaagttaaaggaactgtgcataattgttaagtccagagatagtgtttgccccagaggtcttatcttgctggcttgacttggaaatctaaatttagtacatctctaagtttggtgaggtagaatatgaaggtgctctactttaacataccactggtttgaccttggtagaaagtacttaattacatctcaaggtagctgtgctttttaaaattgagtttgccaaagtagaaacaatgagaaaggaccattataaaacaggatcattgaaggctacatactcttggcttttactctcattctccctattggaaatgtctcttttacctcagggacctggaggtacagcagattataaggataagtacccatatgagcatttggtagtattataggatttattatgaaaataataaaactgcagtaacactggccacagactaacagtacacaggtgcacagttgacaccagggattattgccttgtagagttttgacctttgatgagagagtgttttttacagttgttactgatagcacatttatgtaacttaattgtgctttaaaaatatttaattgtctcttgtgtaataacagtaagtgaaagacgataactaaaattttatataattagatcctggagagaatatttgttgggtgattgaattgaaaataccagtgaatgaaacatacctaaaagggtagataggttgggttggaaagatataccacatcgagggttaattaaatggataagatgtcattatctttttttctttgtaaaggaagattaatgcataaaattattttgtgtaatttacatacaataaaattatgtgttgtacagttgtataatttacatataataaagctaattcaccaattttagatgaagaattcagtacatttggacatatgtttgtagctgtgtaaccaccattgcactcatgatctagaacatttctaacacccccaaaagttccctacttccccttttgcagtcagccttctccctccactgccagcctttggcaaactgatcagtcagtaaagtttcacattatctagaatttcatataaacagaaccatatggtatgtagtctttttaatctggctcctttcactcacatagtgcattggagatgcatccatgttgtagtttattcctttgtattgctgaatagtatcccattatatgtatatgtcagaatttgttgatttaccagttgatgtacatttggattgttttcagtttggggttattatgaataacgcagccatgaacattctagtgcaggtctttatggggacaggagtaggaatgccacatcccgtggtaagtggatgtttaactttttaggaagctgcagaactaatctgcagtggccgtatcattttgcattcccctcagtgatatgtgagagtgcttcagtgactcctatactcaccaacactgggtgtattactgtgacactagatgtattatctattgctacgtaacaacttaccttaaaagctggcagcttaaaacaacagaccctattatcccactttttcaatgggccaagaatcttggctgggcttagctggggcctctggctcagggtcctttacaaggctgcaattaaggtattggccagggctagagtcatctcaaggcttgactagtttttaatttcattttctaatgttttattactagtatatagaaatatagctgaagtgttttgcagggaggctgtataattgaccttgtatcctgcaaccttgctaaactcatttattagttctagaagctcttgggtgtattctctaggattttctacatcaacaaacatggtttctataaatatagttttatgtctttcttacaatcaatacttttttctatctgtattgcattttctagggcttccagtgtggtgttgaatagaagtgttaagagtgaacatccttgcctttttcctgatattggagaaaattcacttgtcttttagcattaagtgtcatgtttgcttttttaaaattttattctatattattttatttttgagacagagtcttgctctgtcacccaggctggagtgcagtggtgtgatctcagctcactacaaccttgacctcctaggctcaagcgatcctcccacctcagcctcctgagtagctgggactgcaggaacatgccaccatgcctggctaatttttgtattttttgtagggatggggttttgccatgttgcccaggctggtcttgaactgttggattcaagcaattcgcctgtctcagcctcccaaagtgctgggattacaggcatgagcctccgtgcctggcctgatatttgcttttttttttttttttaatgctctctattgcagagttggcaaactacaacctgtgacaaatccagcatgccacctgtttttgtaaataaagctttattggagcatagccatgctcattagtttacatcttgtgtatggctgctttaacactacagcagcagagttagagttgtgacacagatagtttggcccataaggcctatatttactgtctaatcttttacaggaaaaatttgccaattcctgccctcttggtttgaggaaattcccttctgttccttgttctgagagtttgtatcatgaatgggtgttaaattttgtcaaatgcattttcaactatgaagggttttgtttttagacgagtgatatgggggactaggtgattgattttctactgttaaaccaaccttgcatctctgggttcaaccccacttggtattatagatttattaccctttttctcttgtggcagattagatctactaaaattttcttgaggatttttgtgtttgtgttcatgagggatattgtagttttttcgtgtctttgccatgttttgggtatcaggataatgctgctgtcattgaggggtgacaaaaatgaggggtggtgtcctttacacttctgttttctggaggatttcatgtagaattggtatgagagtctagcttatggttaaaaacctatgtgtgatgtttcagacctgaccataaacaattacagactttacctaggaggccacatggggaaaagctgccctccctacaccagacttggcgtactgccaatgcattacagtttctaaagggagttgcagtcaaggactcagggccccctgttagtcatgctcttgtaacagtatttgcattgagagtcctggcactttcattcttaggtctctctatctgaggacatgggccaaggtcttcttcaggcacctctgccaaggcctgtttatgcaagaaggagtggaaaaaccttgacatttttttccactgtgactcactacccagtacttttccacccttagcccccttcctttgcacccatacccccaagatccatcaaactgctaaagcctttttttccaagctccttcaacagtgaaccaaccctcatgtctgtgtggatccagctgactcttgactagtgagttgttccttgggaaaaaatggaacagagagagttggtgctttccctggttttagcctcttgcttataccaatgcaatgcctgaaggcttaattcatttttgacttgttgctttgatcagctactccaacacctgacagctcagctctttctcccagctcttgggagatatttttttctttaaatgtttagtagaatataccagtaaggccatctcggccaggagttttctttaatgaaagtttttcactattagttcagttactttagtagacattaacctattcaagtttatctgtgtcttctggaatgagcattggtagtttatgtctttcaagtaatttgttcatttcatctaaattgtcagatttattggtatgaagtgtttatagtattctcttattttactgtccgtagggtctatggtgatgtcctgtctttcattgtagatattgatgtgtcttcttttttctgattattctggccagaggtttatcaattttattgatcttattaaagaatgaactgtttcattgtttttctctatgatttttctgtattctatatcattctttttttattattttattattttatttgctctttatttttctagtttcttaaggtgatggcttacttttatttttttcttatttttttcttttgttgttgttgtttttttaaagaaacagggtcccactcttgctcaggctggagtgcagtggcacgatcatggttcactgcagtctcaaactcctacattcaagctgtcctcccccctcagcctccagagtagttgggattacaggtgcatgccaccatgcctggctaatttttaattttttttgtagagatggggtgttactagttgcccacgctggtctgaaactcctggcctcaagtgatccctccacctctgcctcccaaagtgctgggattccatgtgtaagccactgtgcctggccaaggtgatggcttaaagctattgatttgagatgattccttactttatagtttaagcatataatgccataattttcctcaagcaccgttttagttacgttatacaaattttgaaatgttttgttttcatttcctaatttcccttgtgatttctttattgaaccttggcttatttagaagtatgtttaacttgcagatattggagatttgccagccatctttttgttattaatttctactttaattttgttgtgattagagaacatacattttattaatttaaatttataatttattttaatttataatatggtctgttttacagaatgttgtgtgtgtatttgaaaataatatgaaagctactattattggatggagtgttctataaatgtcagttagattaggttgatcatgctgttctagctttttatatccttattgatttcctcactacttgctctatcaatgactgggaaagtgttgaagtctcccagtatttgtctatttctcctttgattctaccagtgtttgcttaatgtattttgaagctctgttataggtgcatacatgtttatgagtatgttatagatgtattcattttgatatccttctttctctgttactattcctaattctgaatttgactttaatgttattaatataattcttccagccttctcttggttagtcttttcattgcatatctttttctatccttttacttttaatctagctgaatgtagtctttattttgaaagtgcgttccttgttgatagcattattggttcttttttttttttaaatctaatttgacaatctctgtcttttaattggagggtttagacatttgcattgaatgtgattaccaatatagttagatttaaacctacagtcttgctgtttgctttttgtttgtttcattgatcctttgtttcttgtttttttctttttttgctttcctttggatttagtatttttcataattccattttacctccactgttggcttattagctatacttcttcatttcagtattttagtggttgctgtaggatttataataaatatcattaactgaccatatcttcagataatcgtatactacttcatatatagtgtaaaaaccttacaagagtattcactccataatactttgttattgcttttgctttaagtgatcaatgattgtttaaggaaattttttaatgacctttcatgtttattcttttttttttttttccaaaagattcagtattttccgagttttcaaaaactgctggccactcaaagtggatcaacaaaaatttaagagctaaaactgtaaaactcttgaaggctgggcacagaggttcatgcctgtgattccagcactttgagaagctgaggtgggacaatcacttgagcccaggggtttgagaccagcctgggtaacatagaaagaccttgtttctacaaaaaataaaaacacaattagccaggcatggcggtgtgcacctgtagtcccaacttcttgggaggccaaggtggcaggatttcctgagcctgtaagtttgagactgcagtgagctgagttcacgccactgcacttcagcctggacaacagaacaagaccctgtctcaaaaccagaacgaaactataaaactcttagaagaaaacagggctaaatcttcatgactttggatttggcaatggatggttagaattaataccaaaaacacaatcaataaattgataaattggatttaataaaaattaagaacttttgtgtatcaaggacattgtcaagaatgtgaaaagacagcatatagaatggaagaagatatttgcaaatcctatatctgataaaggtttaatatccagaatatgtaaggaactcctgcagctcaacaacagaaagccagttaaatcaattttgaaatgagcaaacgcctgtaaacccagctgcttggcagattgagacaggaggattgcttgaggctaggagttcaagaccaacctggacaacatagtgagaccctgtctaaaaacatttttttaattagctgggtgtggtggcatattcctgtagtcccagctacatgggagaccgaggcaggaggatcacttggggccaggcagtcaaggctgccgtgagctgtgattatgccactgcatcccagcctgggcgacagagtgagaccctgtctgagaaaaaaaaaaaaaaaagaacaaaaaaaaatttagaagattgctattctagtctactattttttcaaagggtggtcttgttaacaattctggagcccacctaaacctgctaaatcaaacttggtagtaaagctggggagatgggcatgtctaacagacgtttctggtggttttgatgtccaggcgtgcagagagatgatgcttaccttgtgttttgtcattattttcaggatttacaccccttccttgtcttttgtatcaatatttatggagtcatgaactctaggataggcatgatgttgagaactaggagttctcccctggccagggagatagaggcaggtctgtggttagttttgtagttggctgtgatgacatctgacatgctctcttcacttgttgtcttcttcctgttcccttgtcaggattatgaagaagctgtgctcttgctgttagaaggagctgcctgggaagaagctttgaggctggtaagaatcttgtaaatcctctggatgttgggtgctaagcagagagagcaagcaagggattccaggtcagttggaatctcttgtcttctgaggttcatgaaataagtagaaataggtcaggttcctggcttaaggaaaagcggtgttactaaaatcatttttatcattcttgataataatttgaaatattactgtcttttactgaaatgaattgaatttccttggctgccttgtaggaggcctgtttttcaggaaaatattctgattacctctgaaagtaatccatgtctttctaagtatcttaactctccagtgactagaagttttccttcctaaaatatcgtgtttttccttctaggtatgcaaatataacagactggatattatagaaaccaacgtaaagccttccattttagaaggtgagggttccattttagatagaattcctcatttggaagaaggtgaggagagagagatgagagagtctcctcctatttactgtgttttcttaataatatgtcatgtagactcaatcaaaattaccacctggatataatatttaattctcactagaatttttaaatatgctgaactattaaatggtaacaaaatatttaaatgttagaaacctgtgatcaaatatgattaagaatctttgtatttggaaatagtaaacttgaatatgaactatattagataataatataacactgataaatttctggcatttaataatcatgttgtggttatataagataatatcctattattctcaagagataaatgctgaaatatttaggaatgaaggatcatatctctgccttactcttaaaaggttccacaaaagtattaatgaatgtgtgtatgcatgcagagaaacaggaagcaaaaaaatgtcaaaatgttagtaattggtaaatcaaagtgaagggtatatgtgtgttcattgaactcttacaacttttatgtaggtttcaacgtttcaaagtattttttaaaagttaccttttcaaatgaagtttgtggttcttagagaacatatgaatattaccagttctagaatactcagatggtcactgtgacctcttaaaagcaaagtggagaaggacatcagtttgacttatagaaaccttagggagtggttgattttaagttctgcatttttatgcacatctaccctgtaagtaacgtctggcctttctgacatttacatgtatgcacattcttaccttgtctgcacccccttcctccatcctaattaaaacgttgctggggtactttttatgtcattcactttaggtacctctaactgggtactgaaaacatcattcctcatctataataatctaaccagctcttacttagattttcaccactaatgagaacctttcttagataaatgccgataattcatctacataggcccaaaacctattaataaaatgcatccttggatagtagtattttgcttttttaaaatgtattctactagtgttatttttctcttgtgtatttttccattggacaatatttattagatacattttttccacatccatgggcattttgatggatgtttagccagaaacatttaggtaattttcttcttatttttgttaactgagctcccctcccctaccccccctttttttgtttgtttgttttgtttgtttgtttgttttgccaatcctcccttgctttaggtatcaagtcttcgttcaggtgattttacaagttcagtggtagcgcatattctgggataatgttgatgaactctaagatctggaatctcagtctctaatttgttaatgcttattaaggaaaaagagctcgcttggaaaacctagtaacctctttctttttgctgaattttaaccctccttcactgctccccgcctttagttttttctctttgcttaaacctcatgctcaaactattttccattctgcatctccagcccagaaaaattatatggcatttctggactctcagacagccacattcagtcgccacaagaaacgtttattggtagttcgagagctcaaggagcaagcccagcaggcaggtctgggtgagtatctgcgtgaaggccatcgacgtgcgggggcagtggggttgggtaacgccacacattgtctagattgcttggtgatccgcctgcaatctgattactgtgccatgggcaagtgtgaggcttctgtggagccccttcagggccctctgtgtctgtgtttgtgtgttggtgaagggcaggaccaagcatgaatggggagagctctgccagacattcccacctacccccattcacccagagcagctgaccacttccgtgtctaacaaaatgagtttcctcatttccagaaaaaagttcaggaaactactgatttacattagtaattactgtatttaatattatctcattcattttgagatcaactttgcaatcattttcatccatcctttgatatgcaccagttgactctagttagttcatttaccgccctgaaagtaaacccacacattagcaggcagtgttttcatcggcttctggttcttcttttctagatgatgaggtaccccacgggcaagagtcagacctcttctctgaaactagcagtgtcgtgagtggcagtgagatgagtggcaaatactcccatagtaactccaggatatcagcgtacgtatcacattgattcagcacattgactatatcctgggcatatagggaaagtggaagcaaatagattggttttctactgggacggtgtagtgggagtggggagaatattcttcagcgctgtgtggaagttgttcagacactttcccagcatatctgagacattaaacttggcattggaaggttttcttcctcagctttgtggcttgtgtgttttcccattccccacgaggcagttcctcccctgaatgctcagtttatattaacatctgattttattttttgaacaaatgttgtgactaaattataggcactgaaaaaatgaaaagataagcttcttcaattcaaaatcaggattggaagagaccataaatgtaaaataagtcataacacttttaccaaatatagtaatttgtcagaaatatttattcagcactcatatggtaggtgcagtagatgttaccaaaaacttataaggagatatgagttataagagtttatagtcttgcttgggatgtgtaaagcaatgcaagattatatattcaaactgaattttgctttaggaatttaaaatggagatctgtgaagttgtgtggggtcatcagcaactgcaagaaagtagccaggcaaggtagcacatgcctgtagtcctagctactcaggaggcttaaaaatatctgtgtaatttctaacaggagatcatccaagaatcgccgaaaagcggagcggaagaagcacagcctcaaagaaggcagtccgctggaggacctggccctcctggaggcactgagtgaagtggtgcagaacactgaaaacctgaaaggtatattctcagtcctgatgatgattcctgaccacaaacaatagtgaataggcagtacagacaggcagagttcagtaggtgattaagctaccattttcccaatttgaggaaagatgagaacttttagcaggaagggtcatgtctgcacacattcctgaagcagcccttcttagctggtaactgagaagccttcctccatttggcatccccctaactgaactgggagagatgcttaagccaggataaagaattgtgggacactgctttctgcgtaggccccccagcgtgcttgattttctttttgtagtacatgtgtttaattattccagcatttgggaagaaaaaagataatgtgggagaaaggacctgcagtgggatcatagaaatttttggctttggatagaagctatgtatgattctgtcaatggagctgggaatataacttaccactctttcaaatttcttctctctagatgaagtataccatattttaaaggtactctttctctttgagtttgatgaacaaggaagggaattacagaaggcctttgaagatacgctgcagttgatggaaaggtcacttccagaaatttggactcttacttaccagcagaattcagctaccccggtaagttttctcagagacggtgtgcatttttttcatcattttcatgggttattgtattcacacaatctccaagtcaaaaagttttcctgttcttaaaacataagatgccatagttaaattatcttagcatttatgtgtaagctgtcagtaagatttgatatttgcctgtagagtgactagtataccttggcataggttaaatggactgtcattttcctttctggatgaagtagctgtcatggagaaaatgggaaagtcacatgattgctcctggccttcaatgaggttggagtggggagagatgggggaagatggggtcagagacggcctctcactttcctttcagaactcagggatgggatcaggctttaaagggaccccaggcaattgcttttccttttgttttatgaaaaatttgacttgtcacttctatgttgttatgatggactttgcgggttgtgtttaaggctgaatcagctttgtatcgcagaattctagtatattgtcatctgtttattatttatacctctgttcactctcttatacttcaagtctattgttaagagtttttatttggattcaaaaaggctggtgtatcagtcaagatctagaaaggaaaacaaaagcctatctattattttatcacagaatttaatatatggatttgttaaataagtattagaggactaaacaaggcaaaagggaaatacagaggaaggacattgagatagtaactgtaggaagcagctttaccctctagctgagggaacaggaggagttgttgggaattattagaatttagaagcctggaagtggggccctgtagagctggctcttgaacctctgagaggagggtgccagccagctaatcctggcatttctgagggagctggttccaagcgtacagaagtaaatggaaactggaaggaacagctgctgctgggggaaaagccagccggtcgggccaggtgtggtggtggctcacgcctgtaatcccagcactttgggaggccaaggcaggcggatcacctgaagtcaggagttcgtgactaatgtggccaacatggagaagccccgtctctactaaaaatacaaaattacccgggcatggtggcgcatgcctgtaatcccagctactcaggaggctgaggcaagagaatcgcttgaacctgggagacagaggttgtgatgagccaagatcgtgccattgtactccaacctgggcagcaagagcgaatctccgtttaaaaaaaaaaaaaaaaaagccagccaatcacggaagaaatctagaaatcttttgttcatcctccagctttgtactccccctctggtgttcactgtaggcaggacatgatgggaagccagcagcaaggaagaatatctttcaggtgcccagccccagcaccacaagcagtggatagaagggtgggttggagctgagagattacaaatcagctcagtgtttagaaacacatacgcttatcatgtcttgatttcctcatttagaaatgggcataagacttctctgtgtgcttcaatagaatgctttgaaggttaaataagagggtgtgtgtaaaagcactttacaaaccgttgaaataaaagcaactaggaatcagggccccagaacttcttgaatttattataataggtatttcttagaagaaatgtgatcatcatcttcaaaactgtagtacttttgaagataattgtttttgttttttgagacagggtctcactctgttgctcaggctggagtgcagtgatcaccgctcactgcagcatccaccgccccgggctcaggtgatcctcccacctcagcctcttgagtagctgggactacaggcgcatgccacaacacctggttaattttcaaattttctgtagagacagggtgtcaccaagttgtccccgctggtcttgaacaactcctgggctcaagtggtctgcccacctcacctctccaaagtgctgggactataggcatcagccaccatgcccggcttgaagataataatttataataccactcccatgagtgatcttctcttctgatcacatattcacattaaggtctattttattttatttttttcttgctctgtcacccaggctagagtgcagtgacagtatgatcaatcatggcttggtgcagcctcgaatgcctgggctaaagcagtcctcccaccgcagtctcctgagtaattgggaccacaggtgcacaccaccatgcccagctaattttaaaattttttcctagacatggggagagggagtcttgctgtgttgcccaagctggtcttgaactcctggcctcaagtgatcctcctgccttggcctcccaaagtgctgagattacaggtgtaagccaccatgcctcccacattaagttctaagacatcaattttatgattgtggttttgattggtgaagtatggttgtggtatgtgcaggataccgtgagtgacttctcatggcattgctcttgagagtgtgccaccaagggtctgcactaaccaggggtgtgcccagaggctcgctgcaggcttgaaattcctgcggagtcttgtgttttacctggagcacatgtgcacagtttccattctgctccatagtatgcacatgtttgtatttatttcaacctaaaaatgtttgtttcccataactctttgcgtataattgatactctacgtatttgtagcctcttttactcttttccctttcctcagggagtggtttgctcatttagaaaaggccaagatatatcactgtagagtttcgtttcttttcttttcctccaccccccatctttaccttgttctgggagaaaggagaattagaagtctgagttgcagctggagaaactggcaaattaaaatcacattgggaaagagaattactgtgtttcacaccataccagtagaaatgacaggctgttttctgctggtagggatttggcctttggtattggcagtcttgagaagtattagataatctttgctgatacagtctattttctcctcaggttctaggtcccaattctactgcaaatagtatcatggcatcttatcagcaacagaagacttcggttcctgttcttggttagtattttttctcatttaatattacaatactaagcagaaggactatctttctgtaagtattgagaagatcagcagtataaggagagattggatacaatttttcactacaaaaaattgactacaattcttcctcaattctaagaccgcatctttagtatgatcagtttcatgcttctagcggtgggggacctggtgcaggaaaatccagcatgaccattgtatgtgtaatttttaaaaatatttatgtggcatatgcttgttcataaaggcacaccacagttccagtttcagtctaaactgtctacatttacatatacatcaaaagattcttctgaagcatcattactggctattggcagttatgctttgcatcttgggggcattttcataaaccttgcttatgagtgggacctttttattatgtttaggattgacaatataatttgaaggcaaatccaaagaatattagcattttatacatatttcctgtttagttatgcatgaagtgttttatttgttgaggggagatgattctcaattagattacttatttccctaaaaattaaaaaccctaagcgctttcttttgaaagttggttagaaacatttgatgagtcagcttgggactttcagtatttgcccttacttatagttggatcaatgaagcatcttagctttgaaaagtgaatgatagtttctaaaataattggcagttttaactgctattatttgcatttctagcatgtgacaagcaactttctgaaattttttttcaccgaagtgctacactgtaatagcattttgatgacatttgaagtagcctgtggggattcaaattaagtttgactttaacagcttatgttgctaccaggaagaacagctaccttccatcccagctaaactcatacatccagactgtaactactgtattcctagctcctcttctgtctagagaatggcaaggttcttttggtatgcagtttcgacatatccacttattcctttttttttcttaagttttttcatttagaaaaaaaaacagatggggtcttaatatgttgcccaggctggtctcagcctcctggtctcaagtgatcctcctgcctcggcctcccaaagtgctgggattacaggcgtctgcccctgtgcccagcccacttatttcccagatgctaggaacttacattagacctgaggccatttggtcattgtttattttgtgctgtagtccaatccagttgtgatttctgcctcctgtgttcctcgttgctggcctgatgctgaccttcaggttaggtcagtcccatcattccccagggtattctagatggctttcccacttcaaagagcactttcttgttttccagctgagccttaaagacactctgtaatatttgagagcccctcattatctgagtgtttattatcattacccttgtggtttcaaggatgtataggaaaaggtaagttcctataattcaaaaattgccactgatgaactaatcacaaaattagtgccactcaaatattactcagctgcccctccccagctaacaatagttaagtatattggcacatccccacaagtgaaatcaatgacttgatgggtcatttctgattgtttcctgctttgatgcaatacaatatcatgcagatcaattgcaagtcttgcaaaaatttagtattacataaaatagattaaaatgatattggaaaagtacttgaatcacagctgggttggacttgttgcaattgatgacaaaataagtgcttcaaatgattttgactatcaaaggattgagagaggtccttagaaaaattgaaaagccctcaagttatttttataaaaatggccttttttgtgtgctgtgaaatccacatatggaaatgtgaaatatgtcatgtcctgctgtcatataatttgtcagaataattactttcttgcccaaaagtctgtactttgtgtttatttcaagttaagtctagaatcaaatatagttgtagttatgcctaattttaaaaaatgagatagagcacattatttttgtaactagtttttttttttttttttcagacagagtcttgctctgtggcccaggcgggagtgcagtggcgcaatctcggctcactgcaagctccgcctcccgggttcacgccattctcctgcctcaccctcctgagtagctgggactacaggcgcccgccatcacgcccggctaatttttttgtatttttagtagagacggggtttcaccgtgttagccaggatggtctcgatctcctgacctcgtgatccacccgcctcggcctcccaaagtgctgggattacaagcgtgagccaccgcgcccggcctgtaaatagtttttttaagataaagtcttattccaactttaattggaatttatgaaataccttgttgatagtgaatttatttaagtagccttttttcagtattgatattcttatatctttatggcaccatttagtggagagaaatgtaaacaaacataaagatgtagtattaaatcataactgcataaaattaactgtagtatgtactgcactactgtaataattttgtagctacctcctgttgctattgtggtgagtgagctcaagtgttaccaatatctgcttaaaatgccatgtgccgctaaccatctccacatgagcagcacatgagagtctccattaattgcatatggcagcgaaaagtgatctcttgcattgtcgtgtattttttatcacgtttaatgtaatatcgtaaaccttaaataacaccatgagacctataggaagtaccacaagtgttgctcccaggaagcagagaaaagtcataacattacaagaaaaagttgacttgctcgatatgtactatagattgaggtctgcagctgtagttgcccaccacttcaagataaatgaacccagtgcaaggactattataaaagaaaaggaaatttatgaagctgtcactgcagttatgccagcaggcatgaaaaccttgtactttttgcaaaatacctttttatgttgtattgaagatgcagcttttatgtgggtgcaggattgctatgagaaaggcatacctatacaactattatgatttgagaaaaagcacagtcattgtatgagaacttaaagcaaaaagatgaaggatcaaagctggagaatttaatgccagcaaaggatggtttgataattttagaaagaggtttggctttgtaaatgtctggataataggaaaagcagctcctgccatccaggaggcagcagcaaaggcagtcaggtttatgatcaggactgcccttatctgtaaagctgctaacccccgagcctggaagggaaaagattaacaccagctgccaggcttttggttgtaccatacaacaagaaggcttggacaaggagaacactttttctggattggttccattgtcgatttgtccctgaagttaagtagtatcttgccagtaaggggactgccttttaaagttcttttgatactggagaatgcccgaggccaccccaaactccatgagttcaacaccgaagacattgaagtgatctacttgcccccaaacacacatctctaattcagcctctagatcagggtgtcataaggacctttaaggctcgttacaaacagtactctatagaaaggattgtcaaatgtatggaaaagaaccttgacagaacatgaaagtctgaaagaattacaccatcaatgatgccatcattgttatagaaaaagctgtgaaagccatcaagcccaggacaataaattcctgctagagaaaactgtgtccagatgtgcatgacttcacaggctttacgacagccaatcaaggaaatcatgaaaaagattgtggatctggcacaaaaaaaaaaaaaaaaaaaaaaaatggtgcatgaaggatttcaagataggaatcttggagaaattcaagaggtgatagacatcacaccggaggaattaacagaagatgacttgatggagatgagtacttccaaaccagcgccagacaatgaggaagattacataaaagaagcagtgccagaaaataaattgacatttgttccaaaggttccaattattcaagactgcctttggcttcttttacaacatggatgattctatgttatgggcactgaaactaaaagaaactgtggaaggattggtaccttagagaaatgaaaaagcaaaaacatcagaaattatggtgtatttctgtaaagttagtgacactgagtgtgcccacctctcttgcctcctctttaacctcccctacctgtttcatctctaccacccctgagacagcaagaccaacccctccacttcctcctctacttcagcctactcaacgtggagatgacaaagatgaagacctttatgatgatccacttccatttaatgaatagtaaatattgttttctttatgattttcttaatattttcttttctctagcttactttattgtaggaatgtagtatataatacatataacatacaaaacatttgttaactgactttttatgctgccaatacactgccgaacaacagtaagctattggtacttgagttttggagattcagaagttaaacatggggccaggtgtggtggctcacacctgtaatcccagcactttgggaggctgaggtgggtggaacgagaccaggagttttgagagtagcctgggcagcatggtgaaaccttgtctctacagaaattagccaggtatggtggtgtacacttgtagtcccagctacttgggaggctgaggcaggagaatcgcttgaacccagggggtcgaggctgcagtgagtcatgatcgtgccactgcactccaacctgggcaacaaaatgagaccctgtctcaaaaaaagaaaaaaaaaaggtatatgcagatttttgactgtgcaggggggtccgcacccataaccctacattcaaggatcaactgtaatttttcatgcctgcatggctcatatgtacagatttactgctggaagtttatcataaataatgctgaaaaagaaaatccttatatatacatattttctcctatctctgcttgcagtatatgattcctggttagaaaagaaacttaacaaatctaagtgaaagagtgcctgggagttttaggttacaatgacagaatcttttcctaaccctctctctccattcactttttttaaagcaggggcatctttattgatcaacatgtttgtcgaagtttcatcataaagtagttcctgtccattaacttcacttactgaatatgtgctatcacattttgctattccttaaaaattgagctagactttacatatagtgaaatgcagagatttcaggtgtacaatttgatgagttttaataaatgtatacagccatgtgactgctgccaccacccctcccaccagtttgaaatacagaacattcttccactttgaatcactgggtgagcatgcctgaggttgaaatgcagtccctcctctcagggcggggcctccaggttgtgtttgctctgacctggaggttgcaggggtagcagacacatgaactctggctctgatggtcttattgctgcaaactccacctgcctagtttgtttagtttagagttactgcctcagcgccctccaacaagagtatgtctgtcacaatttcccttcctttcttgcttttagatgctgagctttttataccaccaaagatcaacagaagaacccagtggaagctgagcctgctagactgagtgactgcagttaggagggatccgacagagaagaccatttccactcattcctgttgtcctaccaccccttgctctttgagggctggctattgagaactggaaagagtaaaatgataacttaccttagcattgccaagaacttcagcagacaacaagcaattctatttattttatgttgtgtatacatcttgatcattagcaagacattaagctttaaccattatggcaccattttgtgagaatgattgttctttcacttgggctgtttgagagcataattatggtaatcatgagattaatgtttcatgatttctacctccaaagtgtgaagacaagtaaaacaatgtttctaaattgtcttattttgttggcggagaagattacaatggctattagtgctacatttggtcaaatgtaatcacttaaatagcttcttgtcaccttaaactaaagcagaataaaaagtatcctttgaaattataagccctcctttgctgacagctattattttgtaacatcttaccaggtcatgtgctttcagttataactgggctgagcctcctataattacaatgtctatagggactgttttactgcctgtgtattttctgctagagagttagcaatgttagagctagaacagattagaatttctaaacagtatcatgcacagttggtgtgagtgatcagtgtgcattgtatggcatgcatggttgtgaattattctctgttctccaaatactgtttctttaactcagatatttttgttagtgtctaggccacttcatttatttttcgtcatggtactttactgacttctctttattcaattctccacgccctcaccaaaaaaaactgtctcaaaatgagaatattttattttcatggtgagtctagaaaacgcccacttcattctgattaaaaattcttccatgttttaaatatcagaaccagacctttcttactgtgtatcttagcccatttgtgtctctataacaacaaccagctttcaaaggaactaatagagtgaaaactcactcattaccacgaggatggcacaagcgattcacgtaggatctgcccctgtgaccaaaacacctcccattgggccccacttccaacactggtgatcacatttcaacatgaggtttagggaaacaaatgcctaaactacagcactgtacataaactaacaggaaatgctgcttttgatcctcaaagaagtgatatagccaaaattgtaatttaagaagcctttcccagtatagcaagatgttaactatagaatcaatctaggagtattcactgtaaaattcaacttttctgtatgtttgaacattttcacaatctcataggagtttttaaaaagaagagaaagaagatatactttgctttggagaaatctactttttgacttacatgggtttgctgtaattaagtgcccaatattgaaaggctgcaagtactttgtaatcactctttggcatgggtaaataagcatggtaacttatattgaaatatagtgctcttgctttggataactgtaaagggacccatgctgatagactggaaatagaagtaaatgtgtttattg

SEQ ID NO: 2 - DNA, 5924 bp, Homo sapiens
ccagtgctggggctgcctagttgacgcacccattgagtcgctggcttctttgcagcgcttcagcgttttcccctggagggcgcctccatccttggaggcctagtgccgtcggagagagagcgggagccgcggacagagacgcgtgcgcaattcggagccgactctgggtgcggactgtgggagctgactctgggtagccggctgcgcgtggctggggaggcgaggccggacgcacctctgtttgggggtcctcagagattaatgattcatcaagggatagttgtactgttctcgtgggaatcacttcatcatgcgaaatctgaaattatttcggaccctggagttcagggatattcaaggtccagggaatcctcagtgcttctctctccgaactgaacaggggacggtgctcattggttcagaacatggcctgatagaagtagaccctgtctcaagagaagtgaaaaatgaagtttctttggtggcagaaggctttctcccagaggatggaagtggccgcattgttggtgttcaggacttgctggatcaggagtctgtgtgtgtggccacagcctctggagacgtcatactctgcagtctcagcacacaacagctggagtgtgttgggagtgtagccagtggtatctctgttatgagttggagtcctgaccaagagctggtgcttcttgccacaggtcaacagaccctgattatgatgacaaaagattttgagccaatcctggagcagcagatccatcaggatgattttggtgaaagcaagtttatcactgttggatggggtaggaaggagacacagttccatggatcagaaggcagacaagcagcttttcagatgcaaatgcatgagtctgctttgccctgggatgaccatagaccacaagttacctggcggggggatggacagttttttgctgtgagtgttgtttgcccagaaacaggggctcggaaggtcagagtgtggaaccgagagtttgctttgcagtcaaccagtgagcctgtggcaggactgggaccagccctggcttggaaaccctcaggcagtttgattgcatctacacaagataaacccaaccagcaggatattgtgttttttgagaaaaatggactccttcatggacactttacacttcccttccttaaagatgaggttaaggtaaatgacttgctctggaatgcagattcctctgtgcttgcagtctggctggaagaccttcagagagaagaaagctccattccgaaaacctgtgttcagctctggactgttggaaactatcactggtatctcaagcaaagtttatccttcagcacctgtgggaagagcaagattgtgtctctgatgtgggaccctgtgaccccataccggctgcatgttctctgtcagggctggcattacctcgcctatgattggcactggacgactgaccggagcgtgggagataattcaagtgacttgtccaatgtggctgtcattgatggaaacagggtgttggtgacagtcttccggcagactgtggttccgcctcccatgtgcacctaccaactgctgttcccacaccctgtgaatcaagtcacattcttagcacaccctcaaaagagtaatgaccttgctgttctagatgccagtaaccagatttctgtttataaatgtggtgattgtccaagtgctgaccctacagtgaaactgggagctgtgggtggaagtggatttaaagtttgccttagaactcctcatttggaaaagagatacaaaatccagtttgagaataatgaagatcaagatgtaaacccgctgaaactaggccttctcacttggattgaagaagacgtcttcctggctgtaagccacagtgagttcagcccccggtctgtcattcaccatttgactgcagcttcttctgagatggatgaagagcatggacagctcaatgtcagttcatctgcagcggtggatggggtcataatcagtctatgttgcaattccaagaccaagtcagtagtattacagctggctgatggccagatatttaagtacctttgggagtcaccttctctggctattaaaccatggaagaactctggtggatttcctgttcggtttccttatccatgcacccagaccgaattggccatgattggagaagaggaatgtgtccttggtctgactgacaggtgtcgctttttcatcaatgacattgaggttgcgtcaaatatcacgtcatttgcagtatatgatgagtttttattgttgacaacccattcccatacctgccagtgtttttgcctgagggatgcttcatttaaaacattacaggccggcctgagcagcaatcatgtgtcccatggggaagttctgcggaaagtggagaggggttcacggattgtcactgttgtgccccaggacacaaagcttgtattacagatgccaaggggaaacttagaagttgttcatcatcgagccctggttttagctcagattcggaagtggttggacaaacttatgtttaaagaggcatttgaatgcatgagaaagctgagaatcaatctcaatctgatttatgatcataaccctaaggtgtttcttggaaatgtggaaaccttcattaaacagatagattctgtgaatcatattaacttgttttttacagaattgaaagaagaagatgtcacgaagaccatgtaccctgcaccagttaccagcagtgtctacctgtccagggatcctgacgggaataaaatagaccttgtctgcgatgctatgagagcagtcatggagagcataaatcctcataaatactgcctatccatacttacatctcatgtaaagaagacaaccccagaactggaaattgtactgcaaaaagtacacgagcttcaaggaaatgctccctctgatcctgatgctgtgagtgctgaagaggccttgaaatatttgctgcatctggtagatgttaatgaattatatgatcattctcttggcacctatgactttgatttggtcctcatggtagctgagaagtcacagaaggatcccaaagaatatcttccatttcttaatacacttaagaaaatggaaactaattatcagcggtttactatagacaaatacttgaaacgatatgaaaaagccattggccacctcagcaaatgtggacctgagtacttcccagaatgcttaaacttgataaaagataaaaacttgtataacgaagctctgaagttatattcaccaagctcacaacagtaccaggatatcagcattgcttatggggagcacctgatgcaggagcacatgtatgagccagcggggctcatgtttgcccgttgcggtgcccacgagaaagctctctcagcctttctcacatgtggcaactggaagcaagccctctgtgtggcagcccagcttaactttaccaaagaccagctggtgggcctcggcagaactctggcaggaaagctggttgagcagaggaagcacattgatgcggccatggttttggaagagagtgcccaggattatgaagaagctgtgctcttgctgttagaaggagctgcctgggaagaagctttgaggctggtatacaaatataacagactggatattatagaaaccaacgtaaagccttccattttagaagcccagaaaaattatatggcatttctggactctcagacagccacattcagtcgccacaagaaacgtttattggtagttcgagagctcaaggagcaagcccagcaggcaggtctggatgatgaggtaccccacgggcaagagtcagacctcttctctgaaactagcagtgtcgtgagtggcagtgagatgagtggcaaatactcccatagtaactccaggatatcagcgagatcatccaagaatcgccgaaaagcggagcggaagaagcacagcctcaaagaaggcagtccgctggaggacctggccctcctggaggcactgagtgaagtggtgcagaacactgaaaacctgaaagatgaagtataccatattttaaaggtactctttctctttgagtttgatgaacaaggaagggaattacagaaggcctttgaagatacgctgcagttgatggaaaggtcacttccagaaatttggactcttacttaccagcagaattcagctaccccggttctaggtcccaattctactgcaaatagtatcatggcatcttatcagcaacagaagacttcggttcctgttcttgatgctgagctttttataccaccaaagatcaacagaagaacccagtggaagctgagcctgctagactgagtgactgcagttaggagggatccgacagagaagaccatttccactcattcctgttgtcctaccaccccttgctctttgagggctggctattgagaactggaaagagtaaaatgataacttaccttagcattgccaagaacttcagcagacaacaagcaattctatttattttatgttgtgtatacatcttgatcattagcaagacattaagctttaaccattatggcaccattttgtgagaatgattgttctttcacttgggctgtttgagagcataattatggtaatcatgagattaatgtttcatgatttctacctccaaagtgtgaagacaagtaaaacaatgtttctaaattgtcttattttgttggcggagaagattacaatggctattagtgctacatttggtcaaatgtaatcacttaaatagcttcttgtcaccttaaactaaagcagaataaaaagtatcctttgaaattataagccctcctttgctgacagctattattttgtaacatcttaccaggtcatgtgctttcagttataactgggctgagcctcctataattacaatgtctatagggactgttttactgcctgtgtattttctgctagagagttagcaatgttagagctagaacagattagaatttctaaacagtatcatgcacagttggtgtgagtgatcagtgtgcattgtatggcatgcatggttgtgaattattctctgttctccaaatactgtttctttaactcagatatttttgttagtgtctaggccacttcatttatttttcgtcatggtactttactgacttctctttattcaattctccacgccctcaccaaaaaaaactgtctcaaaatgagaatatttttattcttcatggtgagtctagaaaacgccccacttcattctgattaaaaaattcttccatgtttttaaatatcagaaccagacctttcttactgtgtatcttagcccatttgtgtctctataacaacaaccagctttcaaaggaactaatagagtgaaaactcactcattaccacgaggatggcacaagcgattcacgtaggatctgcccctgtgaccaaaacacctcccattgggccccacttccaacactggtgatcacatttcaacatgaggtttagggaaacaaatgcctaaactacagcactgtacataaactaacaggaaatgctgcttttgatcctcaaagaagtgatatagccaaaattgtaatttaagaagcctttgtcagtatagcaagatgttaactatagaatcaatctaggagtattcactgtaaaattcaacttttctgtatgtttgaacattttcacaatctcataggagtttttaaaaagaagagaaagaagatatactttgctttggagaaatctactttttgacttacatgggtttgctgtaattaagtgcccaatattgaaaggctgcaagtactttgtaatcactctttggcatgggtaaataagcatggtaacttatattgaaatatagtgctcttgctttggataactgtaaagggacccatgctgatagactggaaatagaagtaaatgtgtttattgaaaaaaaaaaaaaaaa

SEQ ID NO: 3 - protein, 1332 aa, Homo sapiens
MRNLKLFRTLEFRDIQGPGNPQCFSLRTEQGTVLIGSEHGLIEVDPVSREVKNEVSLVAEGFLPEDGSGRIVGVQDLLDQESVCVATASGDVILCSLSTQQLECVGSVASGISVMSWSPDQELVLLATGQQTLIMMTKDFEPILEQQIHQDDFGESKFITVGWGRKETQFHGSEGRQAAFQMQMHESALPWDDHRPQVTWRGDGQFFAVSVVCPETGARKVRVWNREFALQSTSEPVAGLGPALAWKPSGSLIASTQDKPNQQDIVFFEKNGLLHGHFTLPFLKDEVKVNDLLWNADSSVLAVRLEDLQREKSSIPKTCVQLWTVGNYHWYLKQSLSFSTCGKSKIVSLMWDPVTPYRLHVLCQGWHYLAYDWHWTTDRSVGDNSSDLSNVAVIDGNRVLVTVFRQTVVPPPMCTYQLLFPHPVNQVTFLAHPQKSNDLAVLDASNQISVYKCGDCPSADPTVKLGAVGGSGFKVCLRTPHLEKRYKIQFENNEDQDVNPLKLGLLTWIEEDVFLAVSHSEFSPRSVIHHLTAASSEMDEEHGQLNVSSSAAVDGVIISLCCNSKTKSVVLQLADGQIFKYLWESPSLAIKPWKNSGGFPVRFPYPCTQTELAMIGEEECVLGLTDRCRFFINDIEVASNITSFAVYDEFLLLTTHSHTCQCFCLRDASFKTLQAGLSSNHVSHGEVLRKVERGSRIVTVVPQDTKLVLQMPRGNLEVVHHRALVLAQIRKWLDKLMFKEAFECMRKLRINLNPIYDHNPKVFLGNVETFIKQIDSVNHINLFFTELKEEDVTKTMYPAPVTSSVYLSRDPDGNKIDLVCDAMRAVMESINPHKYCLSILTSHVKKTTPELEIVLQKVHELQGNAPSDPDAVSAEEALKYLLHLVDVNELYDHSLGTYDFDLVLMVAEKSQKDPKEYLPFLNTLKKMETNYQRFTIDKYLKRYEKAIGHLSKCGPEYFPECLNLIKDKNLYNEALKLYSPSSQQYQDISIAYGEHLMQEHMYEPAGLMFARCGAHEKALSAFLTCGNWKQALCVAAQLNFTKDQLVGLGRTLAGKLVEQRKHIDAAMVLEESAQDYEEAVLLLLEGAAWEEALRLVYKYNRLDIIETNVKPSILEAQKNYMAFLDSQTATFSRHKKRLLVVRELKEQAQQAGLDDEVPHGQESDLFSETSSVVSGSEMSGKYSHSNSRISARSSKNRRKAERKKHSLKEGSPLEDLALLEALSEVVQNTENLKDEVYHILKVLFLFEFDEQGRELQKAFEDTLQLMERSLPEIWTLTYQQNSATPVLGPNSTANSIMASYQQQKTSVPVLDAELFIPPKINRRTQWKLSLLD

SEQ ID NO: 4 - protein, 1332 aa, Mus musculus
MRNLKLHRTLEFRDIQAPGKPQCFCLRAEQGTVLIGSERGLTEVDPVRREVKTEISLVAEGFLPEDGSGCIVGIQDLLDQESVCVATASGDVIVCNLSTQQLECVGSVASGISVMSWSPDQELLLLATAQQTLIMMTKDFEVIAEEQIHQDDFGEGKFVTVGWGSKQTQFHGSEGRPTAFPVQLPENALPWDDRRPHITWRGDGQYFAVSVVCRQTEARKIRVWNREFALQSTSESVPGLGPALAWKPSGSLIASTQDKPNQQDVVFFEKNGLLHGHFTLPFLKDEVKVNDLLWNADSSVLAIWLEDLPKEDSSTLKSYVQLWTVGNYHWYLKQSLPFSTTGKNQIVSLLWDPVTPCRLHVLCTGWRYLCCDWHWTTDRSSGNSANDLANVAVIDGNRVLVTVFRQTVVPPPMCTYRLLIPHPVNQVIFSAHLGNDLAVLDASNQISVYKCGDKPNMDSTVKLGAVGGNGFKVPLTTPHLEKRYSIQFGNNEEEEEEDFALQLSFLTWVEDDTFLAISYSHSSSQSIIHHLTVTHSEVDEEQGQLDVSSSVTVDGVVIGLCCCSKTKSLAVQLADGQVLKILWESPSLAVEPWKNSEGIPVRFVHPCTQMEVATIGGEECVLGLTDRCRFFILVTEVASNITSFAVCDDFLLVTTHSHTCQGFSLSGASLKMLQAALSGSHEASGEILRKVVWGSRIVTVVPQDTKLILQMPRGNLEVVHHRALVLAQIRKWLDKLMFKEAFECMRKLRINLNLIHDHNPKVFLENVETFVFQIDSVNHINLFFTELREEDVTKTMYPPPITKSVQVSTHPDGKKLDLICDAMRAAMEAINPRKFCLSILTSHVKKTTPELEIVLQKVQELQGNLPFDPESVSVEEALKYLLLLVDVNELFNHSLGTYDFNLVLMVAEKSQKDPKEYLPFLNTLKKMETNYQRFTIDKYLKRYEKALGHLSKCGPEYFTECLNLIKDKNLYKEALKLYRPDSPQYQAVSMAYGEHLMQEHLYEPAGLVFARCGAQEKALEAFLACGSWQQALCVAAQLQMSKDKVAGLARTLAGKLVEQRKHSEAATVLEQYAQDYEEAVLLLLEGSAWEEALRLVYKYDRVDIIETSIKPSILEAQKNYMDFLDSETATFIRHKNRLQVVRALRRQAPQVHVDHEVAHGPESDLFSETSSIMSGSEMSGRYSHSNSRISARSSKNRRKAERKKHSLKEGSPLEGLALLEALSEVVQSVEKLKDEVRAILKVLFLFEFEEQAKELQRAFESTLQLMERAVPEIWTPAGQQSSTTPVLGPSSTANSITASYQQQKTCVPALDAGVYMPPKMDPRSQWKLSLLE

SEQ ID NO: 5 - protein, 1332 aa, Homo sapiens [sequence omitted]

SEQ ID NO: 6 - protein, 1213 aa, Drosophila melanogaster
MRNLKLRYCKELNAVAHPQHLLLQPELNGGASDIYFVVADNKTYAVQESGDVRLKVIADLPDIVGVEFLQLDNAICVASGAGEVILVDPQTGATSEGTFCDVGIESMAWSPNQEVVAFVTRTHNVVLMTSTFDVIAEQPLDAELDPDQQFVNVGWGKKETQFHGSEGKQAAKQKESDSTFTRDEQELNQDVSISWRGDGEFFVVSYVAAQLGRTFKVYDSEGKLNHTAEKSANLKDSVVWRPTGNWIAVPQQFPNKSTIALFEKNGLRHRELVLPFDLQEEPVVQLRWSEDSDILAIRTCAKEEQRVYLYTIGNYHWYLKQVLIFEQADPLALLHWDTRCGAEHTLHVLKESGKHLVYRWAFAVDRNNSIVGVIDGKRLLLTDFDEAIVPPPMSKELQKPIMLMPDAELSGLHLANLTHFSPHYLLATHSSAGSTRLLLLSYKDNDNKPGEWFYRVHSSVRINGLVNAVAVAPYAMNEFYVQTVNNGHTYEVSLKADKTLKVERSYVQLHEPADQIDWVIVKGCIWDGYTGALVTLRNQHLLHIDGYRIGEDVTSFCVVTNYLVYTQLNAMHFVQLDDRRQVASRNIERGAKIVTAVARKARVVLQLPRGNLEAICPRVLVLELVGDLLERGKYQKAIEMSRKQRINLNIIFDHDVKRFVSSVGAFLNDINEPQWLCLFLSELQNEDFTKGMYSSNYDASKQTYPSDYRVDQKVFYVCRLLEQQMNRFVSRFRLPLITAYVKLGCLEMALQVIWKEQQEDASLADQLLQHLLYLVDVNDLYNVALGTYDFGLVLFVAQKSQKDPKEFLPYLNDLKALPIDYRKFRIDDHLKRYTSALSHLAACGEQHYEEALEYIRKHGLYTDGLAFYREHIEFQKNIYVAYADHLRAIAKLDNASLMYERGGQLQQALLSAKHTLDWQRVLVLAKKLSEPLDQVAQSLVGPLQQQGRHMEAYELVKEHCQDRKRQFDVLLEGHLYSRAIYEAGLEDDDVSEKIAPALLAYGVQLESSLQADLQLFLDYKQRLLDIRRNQAKSGEGYIDTDVNLKEVDLLSDTTSLHSSQYSGTSRRTGKTFRSSKNRRKHERKLFSLKPGNPFEDIALIDALHNHVTKIAQQQQPVRDTCKALLQLANAADADPLAAALQREFKTLLQAVDAALDEIWTPELRGNGLMADHLTGPNVDYLALQKEQRYALLSPLKRFKPQLIMMDWQHEILQ

SEQ ID NO: 7 - protein, 1349 aa, Saccharomyces cerevisiae
MVEHDKSGSKRQELRSNMRNLITLNKGKFKPTASTAEGDEDDLSFTLLDSVFDTLSDSITCVLGSTDIGAIEVQQFMKDGSRNVLASFNIQTFDDKLLSFVHFADINQLVFVFEQGDIITATYDPVSLDPAETLIEIMGTIDNGIAAAQWSYDEETLAMVTKDRNVVVLSKLFEPISEYHLEVDDLKISKHVTVGWGKKETQFRGKGARAMEREALASLKASGLVGNQLRDPTMPYMVDTGDVTALDSHEITISWRGDCDYFAVSSVEEVPDEDDETKSIKRRAFRVFSREGQLDSASEPVTGMEHQLSWKPQGSLIASIQRKTDLGEEDSVDVIFFERNGLRHGEFDTRLPLDEKVESVCWNSNSEALAVVLANRIQLWTSKNYHWYLKQELYASDISYVKWHPEKDFTLMFSDAGFINIVDFAYKMAQGPTLEPFDNGTSLVVDGRTVNITPLALANVPPPMYYRDFETPGNVLDVACSFSNEIYAAINKDVLIFAAVPSIEEMKKGKHPSIVCEFPKSEFTSEVDSLRQVAFINDSIVGVLLDTDNLSRIALLDIQDITQPTLITIVEVYDKIVLLSSDFDYNHLVYETRDGTVCQLDAEGQLMEITKFPQLVRDFRVKRVHNTSAEDDDNWSAESSELVAFGITNNGKLFANQVLLASAVTSLEITDSFLLFTTAQHNLQFVHLNSTDFKPLPLVEEGVEDERVRAIERGSILVSVIPSKRSVVLQATRGNLETIYPRIMVLAEVRKNIMAKRYKEAFIVCRTHRINLDILHDYAPELFIENLEVFINQIGRVDYLNLFISCLSEDDVTKTKYKETLYSGISKSFGMEPAPLTEMQIYMKKKMFDPKTSKVNKICDAVLNVLLSNPEYKKKYLQTIITAYASQNPQNLSAALKLISELENSEEKDSCVTYLCFLQDVNVVYKSALSLYDVSLALLVAQKSQMDPREYLPFLQELQDNEPLRRKFLIDDYLGNYEKALEHLSEIDKDGNVSEEVIDYVESHDLYKHGLALYRYDSEKQNVIYNIYAKHLSSNQMYTDAAVAYEMLGKLKEAMGAYQSAKRWREAMSIAVQKFPEEVESVAEELISSLTFEHRYVDAADIQLEYLDNVKEAVALYCKAYRYDIASLVAIKAKKDELLEEVVDPGLGEGFGIIAELLADCKGQINSQLRRLRELRAKKEENPYAFYGQETEQADDVSVAPSETSTQESFFTRYTGKTGGTAKTGASRRTAKNKRREERKRARGKKGTIYEEEYLVQSVGRLIERLNQTKPDAVRVVEGLCRRNMREQAHQIQKNFVEVLDLLKANVKEIYSISEKDRERVNENGEVYYIPEIPVPEIHDFPKSHIVDF

SEQ ID NO: 8 - protein, 1319 aa, Arabidopsis thaliana
MKNLKLFSEVPQNIQLHSTEEVVQFAATDIDQSRLFFASSANFVYALQLSSFQNESAGAKSAMPVEVCSIDIEPGDFITAFDYLAEKESLLIGTSHGLLLVHNVESDVTELVGNIEGGVKCISPNPTGDLLGLITGLGQLIVMTYDWALMYEKALGEVPEGGYVRETNDLSVNCGGISISWRGDGKYFATMGEVYESGCMSKKIKIWESDSGALQSSSETKEFTQGILEWMPSGAKIAAVYKRKSDDSSPSIAFFERNGLERSSFRIGEPEDATESCENLKWNSASDLLAGVVSCKTYDAIRVWFFSNNHWYLKQEIRYPREAGVTVMWDPTKPLQLICWTLSGQVSVRHFMWVTAVMEDSTAFVIDNSKILVTPLSLSLMPPPMYLFSLSFSSAVRDIAYYSRNSKNCLAVFLSDGNLSFVEFPAPNTWEDLEGKDFSVEISDCKTALGSFVHLLWLDVHSLLCVSAYGSSHNKCLSSGGYDTELHGSYLQEVEVVCHEDHVPDQVTCSGFKASITFQTLLESPVLALAWNPSKRDSAFVEFEGGKVLGYASRSEIMETRSSDDSVCFPSTCPWVRVAQVDASGVHKPLICGLDDMGRLSINGKNLCNNCSSFSFYSELANEVVTHLIILTKQDFLFIVDTKDVLNGDVALGNVFFVIDGRRRDEENMSYVNIWERGAKVIGVLNGDEAAVILQTMRGNLECIYPRKLVLSSITNALAQQRFKDAFNLVRRHRIDFNVIVDLYGWQAFLQSAVAFVEQVNNLNHVTEFVCAMKNEDVTETLYKKFSFSKKGDEVFRVKDSCSNKVSSVLQAIRKALEEHIPESPSRELCILTTLARSDPPAIEESLLRIKSVREMELLNSSDDIRKKSCPSAEEALKHLLWLLDSEAVFEAALGLYDLNLAAIVALNSQRDPKEFLPYLQELEKMPESLMHFKIDIKLQRFDSALRNIVSAGVGYFPDCMNLIKKNPQLFPLGLLLITDPEKKLVVLEAWADHLIDEKRFEDAATTYLCCCKLEKASKAYRECGDWSGVLRVGALMKLGKDEILKLAYELCEEVNALGKPAEAAKIALEYCSDISGGISLLINAREWEEALRVAFLHTADDRISVVKSSALECASGLVSEFKESIEKVGKYLTRYLAVRQRRLLLAAKLKSEERSVVDLDDDTASEASSNLSGMSAYTLGTRRGSAASVSSSNATSRARDLRRQRKSGKIRAGSAGEEMALVDHLKGMRMTDGGKRELKSLLICLVTLGEMESAQKLQQTAENFQVSQVAAVELAHDTVSSESVDEEVYCFERYAQKTRSTARDSDAFSWMLKVFISP

SEQ ID NO: 9 - protein, 1178 aa, Caenorhabditis elegans
MKNLQIGSVKTFENPEIAGADDFAVHPILQTIAVSTKNELLLLENNLISSTIKWAEQRRELEVISLSFRTDGNQIVVILADGRALIVEDGEVMDLEIAELTDTTVSAAEWTADEQTLALADNQTLYLADSSLVPFAERPLIFSENERKSAPVNVGWGSESTQFRGSAGKLKPGEKIEKEKEQIEQHSRKTSVHWRWDGEIVAVSFYSSQNDTRNLTVFDRNGEILNNMNIRNIYLSHCFAHKPNANLLCSAIQENGSDDRIVIYERNGETRNSYVVKWPANQIEDRRIIEKIEWNSTGTILSMQTSLGKKHQLEFWHLSNYEFTRKCYWKFSESIIWKWSTVECQNIEVLLESGQFFSVHITPTASFSDVISQNVVVATDELRMYSLCRRVVPPPMCDYSIQCLSDIVAYTTSTHHVHVITSDWKIISCMLFFKKKKRNYSNPFFRKKYILEILKVPSHKTYFACFAVSQDTDGYKFNSDRASIDEVLHTEVTEGIICGFVYDEPSESYIIWNVSHGKHQISRVGANPEKIFEGENIGWIGVNPSNKHVEIASNDGKFIDLNTKEELFKIDKFESTEVHFIQVCHGILNHHVIQVDNSMLFLDSERVSQDAISILTRGSDILLIDFDNKLRFIDAESGKTLEDVRNVEAGCELVACDSQSANVILQAARGNLETIQPRRYVMAHTRDLLDRKEYIASFKWMKKHRVDMSFAMKYKGDDLEDDIPIWLKTSNDSQFLEQLLISCTEVFEDAGSSLCMTVARYVRDLSDAEKTKMFPLLLTALLSARSKPSKVNDCLKEVQEHVEKIADRKDVFTRNSLHHISFFVPAKELFNCALSTYDLKLAQQVAEASNYDPKEYLPVLNKLNRVMCTLERQYRINVVREAWIDAVSSLFLLDSSKERGSEETWWNDIEDIIIQREKLYQDALTLVKPGDRRYKQCCELYAELERKVHWREAALFYELSGNSEKTLKCWEMSRDVDGLAASARRLAVDAGKLKIHAIKMSTTLREARQPKELAKALKLAGSSSTQIVHVLCDAFEWLDASREVEVGKEEALKKAALSRNDQVLMDLERRKTEFENYKKRLAVVRENKLKRVEQFAAGEVDDLRDDISVISSISSRSGSSKVSMASTVRRKQIEKKKSSLKEGGEYEDSALLNVLSENYRWLENIGSEFCFPWNFNIL

SEQ ID NO: 10 - DNA, 17 bp, Mus sp.
ttttttttccctcagaa

SEQ ID NO: 11 - DNA, 17 bp, Mus sp.
tatgctttgtgaaaggt

SEQ ID NO: 12 - DNA, 17 bp, Mus sp.
ttttctctgatgcagct

SEQ ID NO: 13 - DNA, 17 bp, Mus sp.
acatgaactcctaagct

SEQ ID NO: 14 - DNA, 17 bp, Mus sp.
cttgaaaaactgtaggc

SEQ ID NO: 15 - DNA, 17 bp, Mus sp.
ggtgtctctcttcagcc

SEQ ID NO: 16 - DNA, 17 bp, Mus sp.
ctacctcctttgcagag

SEQ ID NO: 17 - DNA, 17 bp, Mus sp.
aggttctgctttcagac

SEQ ID NO: 18 - DNA, 17 bp, Mus sp.
ttttgtccctaccaggt

SEQ ID NO: 19 - DNA, 17 bp, Mus sp.
tccctccacacacagtc

SEQ ID NO: 20 - DNA, 17 bp, Mus sp.
cttttcattgtgtagac

SEQ ID NO: 21 - DNA, 17 bp, Mus sp.
ttttttgttttctaggt

SEQ ID NO: 22 - DNA, 17 bp, Mus sp.
ctaatatttgaacagga

SEQ ID NO: 23 - DNA, 17 bp, Mus sp.
ttttttttgctttagtt

SEQ ID NO: 24 - DNA, 17 bp, Mus sp.
ttaatcttacaacagag

SEQ ID NO: 25 - DNA, 17 bp, Mus sp.
ttcatttctttgcagga

SEQ ID NO: 26 - DNA, 17 bp, Mus sp.
tcttgcctgttgcaggt

SEQ ID NO: 27 - DNA, 17 bp, Mus sp.
cactggtatttttagtg

SEQ ID NO: 28 - DNA, 17 bp, Mus sp.
gggttttattttgagat

SEQ ID NO: 29 - DNA, 17 bp, Mus sp.
ttcctgtcctcacagac

SEQ ID NO: 30 - DNA, 17 bp, Mus sp.
tactttctttgataggt

SEQ ID NO: 31 - DNA, 17 bp, Mus sp.
tactgtggttcttaggg

SEQ ID NO: 32 - DNA, 17 bp, Mus sp.
cacttactacctcaggt

SEQ ID NO: 33 - DNA, 17 bp, Mus sp.
cttaaactccaacagga

SEQ ID NO: 34 - DNA, 17 bp, Mus sp.
aacttttttcctaggga

SEQ ID NO: 35 - DNA, 17 bp, Mus sp.
ttttttttttttcagga

SEQ ID NO: 36 - DNA, 17 bp, Mus sp.
cgtctcttgtcacaggc

SEQ ID NO: 37 - DNA, 17 bp, Mus sp.
ttgctgtcttttcagga

SEQ ID NO: 38 - DNA, 17 bp, Mus sp.
ctcttcccttgtcagga

SEQ ID NO: 39 - DNA, 17 bp, Mus sp.
tttcttccctcttaggt

SEQ ID NO: 40 - DNA, 17 bp, Mus sp.
attatgcatcctcagcc

SEQ ID NO: 41 - DNA, 17 bp, Mus sp.
gttcatcttctctagat

SEQ ID NO: 42 - DNA, 17 bp, Mus sp.
tgtaatttctgacagga

SEQ ID NO: 43 - DNA, 17 bp, Mus sp.
ccatttcttctctagat

SEQ ID NO: 44 - DNA, 17 bp, Mus sp.
ctgttttctgcttaggt

SEQ ID NO: 45 - DNA, 17 bp, Mus sp.
cattcttgcttccagat

SEQ ID NO: 46 - DNA, 17 bp, Mus sp.
aggtgagcattcgcccg

SEQ ID NO: 47 - DNA, 17 bp, Mus sp.
aagtaggtcactgatgc

SEQ ID NO: 48 - DNA, 17 bp, Mus sp.
aggtaggtgtaaggcct

SEQ ID NO: 49 - DNA, 17 bp, Mus sp.
aggtaagctttgcactg

SEQ ID NO: 50 - DNA, 17 bp, Mus sp.
aggtaagcgtttcttgg

SEQ ID NO: 51 - DNA, 17 bp, Mus sp.
tggtaaggcgggatgat

SEQ ID NO: 52 - DNA, 17 bp, Mus sp.
tggtgtctctcttcagc

SEQ ID NO: 53 - DNA, 17 bp, Mus sp.
aagtgagtgagcataaa

SEQ ID NO: 54 - DNA, 17 bp, Mus sp.
aggtaggggtcagagtt

SEQ ID NO: 55 - DNA, 17 bp, Mus sp.
tggtatgacagcttgtg

SEQ ID NO: 56 - DNA, 17 bp, Mus sp.
aagtaagttgctgcgaa

SEQ ID NO: 57 - DNA, 17 bp, Mus sp.
tggtaagtggaagcagg

SEQ ID NO: 58 - DNA, 17 bp, Mus sp.
tcgtaagttcctaaata

SEQ ID NO: 59 - DNA, 17 bp, Mus sp.
aggtatcatggttcatc

SEQ ID NO: 60 - DNA, 17 bp, Mus sp.
gggtgaggatcagagtt

SEQ ID NO: 61 - DNA, 17 bp, Mus sp.
aggtgaatagacacggc

SEQ ID NO: 62 - DNA, 17 bp, Mus sp.
aggtatgtaggcttggt

SEQ ID NO: 63 - DNA, 17 bp, Mus sp.
aagtaagctctcctata

SEQ ID NO: 64 - DNA, 17 bp, Mus sp.
aggtaagctgactcttc

SEQ ID NO: 65 - DNA, 17 bp, Mus sp.
aagtaagtatttattct

SEQ ID NO: 66 - DNA, 17 bp, Mus sp.
aggtacactttgcgtct

SEQ ID NO: 67 - DNA, 17 bp, Mus sp.
aggtaagtattttgata

SEQ ID NO: 68 - DNA, 17 bp, Mus sp.
aagtgggtgctgtgtgt

SEQ ID NO: 69 - DNA, 17 bp, Mus sp.
aggtagagacctgcgcg

SEQ ID NO: 70 - DNA, 17 bp, Mus sp.
aggtatgtggagttgag

SEQ ID NO: 71 - DNA, 17 bp, Mus sp.
tggtaagggtttttttt

SEQ ID NO: 72 - DNA, 17 bp, Mus sp.
aggtatgtggtgggtta

SEQ ID NO: 73 - DNA, 17 bp, Mus sp.
aggtaagcagggccatt

SEQ ID NO: 74 - DNA, 17 bp, Mus sp.
aggtgagctcctccccg

SEQ ID NO: 75 - DNA, 17 bp, Mus sp.
tggtaaggaagctctga

SEQ ID NO: 76 - DNA, 17 bp, Mus sp.
aggtgaggattacattt

SEQ ID NO: 77 - DNA, 17 bp, Mus sp.
gggtgagtgcctccaaa

SEQ ID NO: 78 - DNA, 17 bp, Mus sp.
gcgtacgtacgagacct

SEQ ID NO: 79 - DNA, 17 bp, Mus sp.
aggtatggcttcagtgc

SEQ ID NO: 80 - DNA, 17 bp, Mus sp.
cggtaagcttcctcaga

SEQ ID NO: 81 - DNA, 17 bp, Mus sp.
cggtgtactgctcgttc

SEQ ID NO: 82 - DNA, 19 bp, Artificial Sequence
  Description of Artificial Sequence Primer
gccagtgtttttgcctgag

SEQ ID NO: 83 - DNA, 19 bp, Artificial Sequence
  Description of Artificial Sequence Primer
cggattgtcactgttgtgc

SEQ ID NO: 84 - DNA, 20 bp, Artificial Sequence
  Description of Artificial Sequence Primer
gactgctctcatagcatcgc

SEQ ID NO: 85 - DNA, 15 bp, Artificial Sequence
  Description of Artificial Sequence Primer
aagtaagygccattg

SEQ ID NO: 86 - DNA, 15 bp, Artificial Sequence
  Description of Artificial Sequence Primer
ggttcacsgattgtc

SEQ ID NO: 87 - DNA, 17 bp, Artificial Sequence
  Description of Artificial Sequence Primer
ggcgtcgtagaaattgc

SEQ ID NO: 88 - DNA, 20 bp, Artificial Sequence
  Description of Artificial Sequence Primer
gtggtgctgaaggggcaggc

SEQ ID NO: 89 - DNA, 10 bp, Artificial Sequence
  Description of Artificial Sequence Primer
tacagactta